(19) United States
(12) Patent Application Publication
(10) Pub. No.: US 2026/0083467 A1
(43) Pub. Date: Mar. 26, 2026

(54) CLOT ASPIRATION AND BLOOD REINFUSION SYSTEMS, AND ASSOCIATED DEVICES AND METHODS

(71) Applicant: Inari Medical, Inc., Irvine, CA (US)

(72) Inventors: Jared Shimizu, Tustin, CA (US); Donald Joseph Fuller, San Clemente, CA (US); Parker Ozenne, Aliso Viejo, CA (US); Anna Kristine Kratochvil, Huntington Beach, CA (US); Jakub Marek Truty, San Clemente, CA (US)

(21) Appl. No.: 19/334,624

(22) Filed: Sep. 19, 2025

Related U.S. Application Data
(60) Provisional application No. 63/697,992, filed on Sep. 23, 2024.
(60) Provisional application No. 63/761,793, filed on Feb. 21, 2025.

(51) Int. Cl. A61B 17/22; A61B 17/00; A61M 1/00; A61M 39/00; A61M 39/24

(52) U.S. Cl. CPC A61B 17/22; A61M 1/74; A61M 39/24; A61B 2017/00561; A61B 2217/005; A61M 2039/0009; A61M 2039/248; A61M 2202/0021; A61M 2202/0413; A61M 2205/3331; A61M 2205/7545; A61M 2210/12

(57) ABSTRACT

Disclosed herein are devices, systems, and methods for aspirating clot material and blood from the vasculature of a patient, filtering the blood from the clot material, and returning the filtered blood to the vasculature of the patient. In some embodiments, a system in accordance with the present technology can include (i) an aspiration catheter configured to be positioned within the vasculature of a patient proximate to clot material therein, (ii) a reinfusion catheter configured to be positioned within the vasculature, (iii) a collection chamber selectively fluidly coupled to the aspiration catheter and the reinfusion catheter, (iv) a pump assembly selectively fluidly coupled to the collection chamber and configured to generate positive and negative pressure within the collection chamber, and (v) a control system configured to selectively control the fluid couplings to perform clot aspiration and filtered blood reinfusion operations.

### CROSS-REFERENCE TO RELATED APPLICATIONS

This application claims the benefit of (i) U.S. Provisional Patent Application No. 63/761,793, filed Feb. 21, 2025, and titled “CLOT ASPIRATION AND BLOOD REINFUSION SYSTEMS, AND ASSOCIATED DEVICES AND METHODS,” and (ii) U.S. Provisional Patent Application No. 63/697,992, filed Sep. 23, 2024, and titled “CLOT ASPIRATION AND BLOOD REINFUSION SYSTEMS, AND ASSOCIATED DEVICES AND METHODS,” each of which is incorporated herein by reference in its entirety.

### TECHNICAL FIELD

The present technology generally relates to systems, devices, and methods for (i) aspirating clot material and blood from the vasculature of a patient, (ii) filtering the blood from the clot material, and (iii) returning the filtered blood to the vasculature of the patient, and associated devices and methods.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by occlusive material, such as clot material, tissue ischemia develops. The ischemia will progress to tissue infarction if the occlusion persists. However, infarction does not develop or is greatly limited if the flow of blood is reestablished rapidly. Failure to reestablish blood flow can accordingly lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT commonly occurs where there is a propensity for stagnated blood (e.g., long-distance air travel, immobility, etc.) and clotting (e.g., cancer; recent surgery, such as orthopedic surgery, etc.). DVT can obstruct drainage of venous blood from the legs, leading to swelling, ulcers, pain, and infection. DVT can also create a reservoir in which blood clots can collect and then travel to other parts of the body, including the heart, lungs, brain (which may cause a stroke), abdominal organs, and/or extremities.

In the pulmonary circulation, occlusive material can cause harm by obstructing pulmonary arteries-a condition known as pulmonary embolism. If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood, resulting in low blood oxygen and buildup of blood carbon dioxide.

Various systems exist for performing thrombectomies or removing occlusive material to reestablish blood flow within a patient. Some such systems can utilize aspiration to remove occlusive material and blood from the patient and can include components for filtering the occlusive material from the blood and returning the filtered blood to the patient. However, such systems are often cumbersome and/or difficult to operate for a surgeon and/or other surgical team members.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially-schematic side cross-sectional view of a clot treatment system in accordance with embodiments of the present technology.

FIG. 2A is a partially-schematic side view of an aspiration catheter and a reinfusion catheter of the clot treatment system of FIG. 1 with the reinfusion catheter configured as an introducer in accordance with embodiments of the present technology.

FIG. 2B is a partially-schematic side view of a multi-lumen catheter configured as both the aspiration catheter and the reinfusion catheter of FIG. 1 in accordance with embodiments of the present technology.

FIG. 3 is a perspective view of a collection chamber of the clot treatment system of FIG. 1 in accordance with embodiments of the present technology.

FIG. 4 is a perspective view of a proximal portion of an aspiration catheter of the clot treatment system of FIG. 1 in accordance with embodiments of the present technology.

FIGS. 5A-5H are partially-schematic side cross-sectional views of the clot treatment system of FIG. 1 illustrating a method or sequence performed by the system of aspirating clot material and blood from a patient, filtering the blood from the clot material, and reinfusing the filtered blood into the patient in accordance with embodiments of the present technology.

FIGS. 6A-6C are a perspective view, a partially-transparent perspective view, and a partially-transparent side view, respectively, of the system of FIG. 1 in accordance with embodiments of the present technology.

FIG. 7A is a side cross-sectional view of a clot treatment system in accordance with additional embodiments of the present technology.

FIG. 7B is a perspective of a reinfusion manifold and a portion of a reinfusion conduit including a reinfusion valve of the clot treatment system of FIG. 7A in accordance with embodiments of the present technology.

FIG. 7C is a side cross-sectional view of the clot treatment system of FIGS. 7A and 7B during a reinfusion state of the clot treatment system in accordance with additional embodiments of the present technology.

FIG. 8 is a side cross-sectional view of a clot treatment system in accordance with additional embodiments of the present technology.

FIG. 9 is a side view of an automated stopcock valve in accordance with embodiments of the present technology.

FIG. 10 is a perspective view of an automated stopcock valve in accordance with additional embodiments of the present technology.

FIG. 11 is a perspective view of a portion of a clot treatment system in accordance with additional embodiments of the present technology.

FIG. 12 is another perspective view of the portion of the clot treatment system of FIG. 11 including a different valve assembly that comprises a duckbill check-valve in accordance with additional embodiments of the present technology.

FIG. 13 is a perspective view of the portion of the clot treatment system of FIG. 11 including a collection component and a housing having different geometries in accordance with embodiments of the present technology.

FIG. 14 is a side view of a portion of a clot treatment system in accordance with additional embodiments of the present technology.

FIG. 15 is another side view of the portion of the clot treatment system of FIG. 14 in which a first chamber is spaced apart from a second chamber and connected thereto via tubing in accordance with additional embodiments of the present technology.

FIG. 16 is another side view of the portion of the clot treatment system of FIG. 14 in which the first chamber is positioned side-by-side with the second chamber and connected thereto via the tubing in accordance with additional embodiments of the present technology.

FIG. 17 is a side cross-sectional view of a clot treatment system in accordance with additional embodiments of the present technology.

FIG. 18 is a side cross-sectional view of a portion of a clot treatment system in accordance with additional embodiments of the present technology.

FIGS. 19A-19D are side views of a collection component of the system of FIG. 18 in a resting state, a charged vacuum state, a filled with vacuum state, and a vented state, respectively, during operation system of FIG. 18 in accordance with embodiments of the present technology.

FIGS. 20A and 20B are a rear view and a side view, respectively, of a collection component that can be used in the clot treatment system of FIG. 18 in accordance with additional embodiments of the present technology.

FIG. 21 is a perspective view of a portion of a clot treatment system in accordance with additional embodiments of the present technology.

FIG. 22 is a side cross-sectional view of a portion of a clot treatment system in accordance with additional embodiments of the present technology.

FIG. 23 is another side cross-sectional view of the portion of the clot treatment system of FIG. 22 illustrating the flow of clot material and blood through a collection chamber during aspiration in accordance with embodiments of the present technology.

FIG. 24 is another side view of the portion of the clot treatment system of FIG. 22 in which a first chamber is spaced apart from a second chamber and connected thereto via tubing and an aspiration valve in accordance with additional embodiments of the present technology.

FIGS. 25A and 25B are a side cross-sectional view and a perspective view, respectively, of a portion of a clot treatment system in accordance with additional embodiments of the present technology.

FIG. 26 is a side cross-sectional view of a portion of a clot treatment system in accordance with additional embodiments of the present technology.

FIG. 27 is a side view of a conical coarse filter in accordance with embodiments of the present technology.

FIGS. 28A and 28B are side cross-sectional views of a duckbill valve in a closed position and an open position, respectively, that can be utilized in the system of FIG. 11 and/or other systems described herein in accordance with embodiments of the present technology.

FIGS. 29A and 29B are side cross-sectional views of an umbrella valve in a closed position and an open position, respectively, that can be utilized in the system of FIG. 11 and/or other systems described herein in accordance with embodiments of the present technology.

FIGS. 30A and 30B are side cross-sectional views of a cross-slit valve in a closed position and an open position, respectively, that can be utilized in the system of FIG. 11 and/or other systems described herein in accordance with embodiments of the present technology.

FIGS. 31A and 31B are side cross-sectional views of a ball valve in a closed position and an open position, respectively, that can be utilized in the system of FIG. 11 and/or other systems described herein in accordance with embodiments of the present technology.

FIG. 32 is a side cross-sectional view of a dome valve in an open position that can be utilized in the system of FIG. 11 and/or other systems described herein in accordance with embodiments of the present technology.

FIG. 33 is a side cross-sectional view of a clot treatment system in accordance with additional embodiments of the present technology.

FIG. 34 is a partially-schematic side view of a portion of a clot treatment system in accordance with additional embodiments of the present technology.

FIG. 35 is an enlarged, partially-schematic side view of the portion of the clot treatment system of FIG. 34 in accordance with additional embodiments of the present technology.

FIG. 36 is a perspective rear view of the portion of the clot treatment system of FIG. 34 in accordance with additional embodiments of the present technology.

FIGS. 37A and 37B are side views of the portion of the clot treatment system of FIG. 34 in accordance with additional embodiments of the present technology.

FIG. 38 is a side view of a remote that can be utilized in the system of FIG. 34 and/or other systems described herein in accordance with embodiments of the present technology.

FIG. 39 is a partially-schematic top view of the portion of the clot treatment system of FIG. 34 in accordance with additional embodiments of the present technology.

FIGS. 40A and 40B are top views of the mode selector of the clot treatment system of FIG. 34 in accordance with additional embodiments of the present technology.

FIGS. 41A-41F are simplified side views of the portion of the clot treatment system of FIG. 34 during different aspiration and reinfusion states in accordance with additional embodiments of the present technology.

### DETAILED DESCRIPTION

The present technology is generally directed to systems, devices, and methods for aspirating clot material and blood from the vasculature of a patient, filtering the blood from the clot material, and returning the filtered blood to the vasculature of the patient, and associated devices and methods. In some embodiments, a system in accordance with the present technology can include (i) an aspiration catheter configured to be positioned within the vasculature of a patient proximate to clot material therein, (ii) a reinfusion catheter configured to be positioned within the vasculature, (iii) a collection chamber selectively fluidly coupled to the aspiration catheter and the reinfusion catheter and having a filter therein, (iv) a pump assembly selectively fluidly coupled to the collection chamber and configured to generate positive and negative pressure within the collection chamber, and (v) a control system. The control system can control the fluid couplings between the aspiration catheter, the reinfusion catheter, the collection chamber, and the pump assembly to cause the pump assembly to generate vacuum pressure within the collection chamber to aspirate blood and clot material therein through the aspiration catheter and to generate positive pressure within the collection chamber to drive the blood through the filter and through the reinfusion catheter for reinfusion to the vasculature.

More specifically, the pump assembly can include a pump having an inlet and an outlet. The pump can be configured to draw air through the inlet and drive the air out of the outlet. The aspiration catheter can be selectively fluidly coupled to the collection chamber by an aspiration valve between the aspiration catheter and the collection chamber. The aspiration valve can be movable between an open position in which the aspiration lumen is fluidly connected to the collection chamber and a closed position in which the aspiration lumen is fluidly disconnected from the collection chamber. The reinfusion catheter can be selectively fluidly coupled to the collection chamber by a reinfusion valve between the reinfusion catheter and the collection chamber. The reinfusion valve can be movable between an open position in which the reinfusion lumen is fluidly connected to the collection chamber and a closed position in which the reinfusion lumen is fluidly disconnected from the collection chamber. The inlet of the pump can be selectively fluidly coupled to the collection chamber by a vacuum valve between the pump assembly and the collection chamber. The vacuum valve can be movable between an open position in which the inlet of the pump is fluidly connected to the collection chamber, such that the pump generates negative pressure within the collection chamber and a closed position in which the inlet of the pump is fluidly disconnected from the collection chamber. The outlet of the pump can be selectively fluidly coupled to the collection chamber by a positive pressure valve between the pump assembly and the collection chamber. The positive pressure valve can be movable between an open position in which the outlet of the pump is fluidly connected to the collection chamber, such that the pump generates positive pressure within the collection chamber and a closed position in which the outlet of the pump is fluidly disconnected from the collection chamber.

During a clot treatment procedure, the control system can control each of the vacuum valve, the positive pressure valve, the aspiration valve, and the reinfusion valve in a sequence/cycle to aspirate clot material and blood into the collection chamber and drive the blood out of the chamber through the filter into the reinfusion catheter. For example, the control system can initially close each of the vacuum valve, the positive pressure valve, the aspiration valve, and the reinfusion valve. The control system can then open the vacuum valve such that the pump generates negative pressure within the collection chamber, and close the vacuum valve after a sufficient level of vacuum is generated within the collection chamber. The control system can then open the aspiration valve to apply the negative pressure to the aspiration catheter to aspirate at least a portion of the clot material and the blood through the aspiration lumen into the collection chamber, and close the aspiration valve after a sufficient amount of clot material is aspirated into the collection chamber and/or a selected level of vacuum pressure is applied to the aspiration catheter. The control system can then open the positive pressure valve and the reinfusion valve such that the pump generates positive pressure within the chamber to drive at least a portion of the blood from the collection chamber through the filter and into and at least partially through the reinfusion catheter for reinfusion into the vasculature of the patient. The filter is configured to inhibit the portion of the clot material from passing through the filter to the reinfusion catheter.

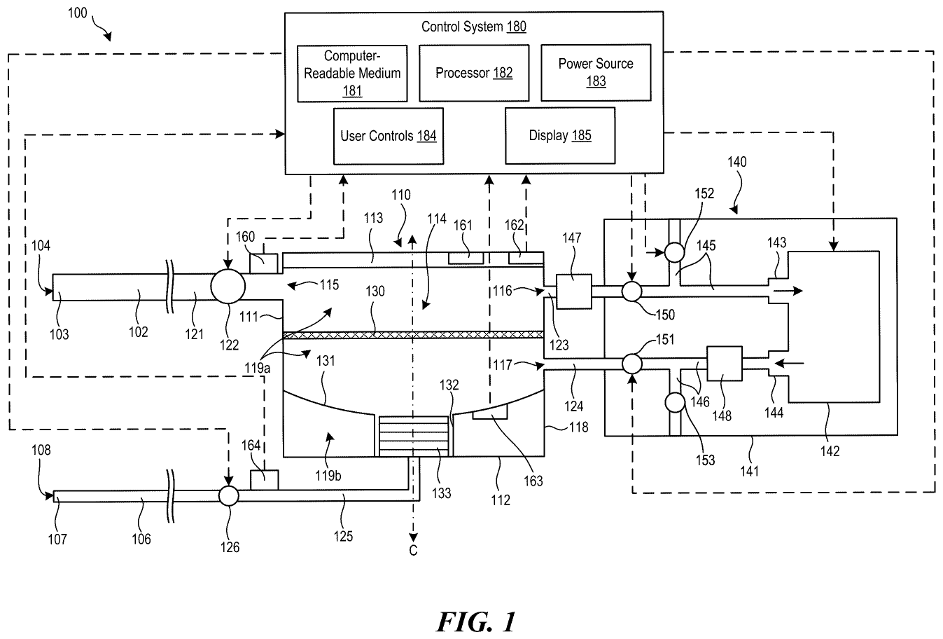

Certain details are set forth in the following description and in FIGS. 1-41F to provide a thorough understanding of various embodiments of the present technology. In other instances, well-known structures, materials, operations, and/or systems often associated with intravascular procedures, clot removal procedures, clot treatment systems, clot treatment devices, fluid control devices, electromechanical actuators, vacuum pumps, catheters, blood filtering devices, and/or the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. Moreover, although many of the devices and systems are described herein in the context of removing and/or treating clot material, the present technology can be used to remove and/or treat other unwanted material in addition to or alternatively to clot material, such as thrombi, emboli, plaque, intimal hyperplasia, post-thrombotic scar tissue, etc. Accordingly, the terms “clot” and “clot material” as used herein can refer to any of the foregoing materials and/or the like.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

In the Figures, identical reference numbers identify identical, or at least generally similar, elements. To facilitate the discussion of any particular element, the most significant digit or digits of any reference number refers to the Figure in which that element is first introduced. For example, collection chamber 110 is first introduced and discussed with reference to FIG. 1.

The headings provided herein are for convenience only and should not be construed as limiting the subject matter disclosed. To the extent any materials incorporated herein by reference conflict with the present disclosure, the present disclosure controls.

### I. SELECTED EMBODIMENTS OF CLOT TREATMENT SYSTEMS, DEVICES, AND METHODS

FIG. 1 is a partially-schematic side cross-sectional view of a clot treatment system 100 (“system 100”) in accordance with embodiments of the present technology. The system 100 can also be referred to as an aspiration assembly, a vascular access system, a clot removal system, a thrombectomy system, an aspiration thrombectomy and blood reinfusion system, an automated aspiration thrombectomy and blood reinfusion system, and/or the like. In the illustrated embodiment, the system 100 includes (i) an aspiration catheter 102 configured to be positioned within the vasculature of patient proximate to clot material within the vasculature, (ii) a collection chamber 110 selectively fluidly couplable to the aspiration catheter 102, (iii) a pump assembly 140 selectively fluidly couplable to the collection chamber 110, (iv) a reinfusion catheter 106 selectively fluidly couplable to the collection chamber 110 and configured to be positioned within the vasculature of the patient, and (vi) a control system 180 (e.g., a computing device, an electronics system, an electronics subsystem, a control system, control assembly, a controller, and/or the like) communicatively coupled to one or more components of the aspiration catheter 102, the collection chamber 110, the pump assembly 140, and/or the reinfusion catheter 106.

In general, the control system 180 is configured to control the system 100 (i) generate vacuum pressure (e.g., negative pressure) within the collection chamber 110 via the pump assembly 140 and apply the vacuum pressure to the aspiration catheter 102 to aspirate clot material and blood from the vasculature of the patient into collection chamber 110 and (ii) generate positive pressure within the collection chamber 110 via the pump assembly 140 to drive/force the aspirated blood from the collection chamber 110 into the reinfusion catheter 106. The collection chamber 110 is configured to filter the clot material from the blood and to permit the filtered blood to pass into the reinfusion catheter 106 while the clot material remains in the collection chamber 110. In some aspects of the present technology, the system 100 enables an at least partially automated system for clot removal and blood reinfusion that reduces blood loss for the patient, reduces complications and complexity for an operator (e.g., surgeon, surgical team member), reduces procedure times, increases procedure efficacy, and/or the like.

More specifically, the aspiration catheter 102 can be an elongate member (e.g., a sheath, a shaft) defining an aspiration lumen 104 and configured to be inserted into and through a patient's vasculature and used to, for example, remove or otherwise treat clot material therein. The aspiration catheter 102 can have a distal end portion 103 configured to be positioned proximate to the clot material within the vasculature, such as proximal to, within, and/or distal to the clot material within the vasculature. The aspiration catheter 102 can be a large bore catheter having, for example, a size equal to or greater than 16 French, such as 18 French, 20 French, 22 French, 24 French, 26 French, 28 French, 30 French, 32 French, and/or the like, and a corresponding inner diameter across an inner surface defining the aspiration lumen 104. In some embodiments, the clot material comprises a pulmonary embolism within a pulmonary artery of the patient, a deep vein thrombosis (DVT) within a peripheral vein of the patient, and/or the like. The aspiration catheter 102 can have varying lengths, flexibilities, shapes, thicknesses, and/or other properties along its length. For example, the aspiration catheter 102 can comprise one or more coils, braids, and/or other structures positioned between one or more liner layers (e.g., an inner liner layer and an outer liner layer). In some embodiments, the aspiration catheter 102 can include several features generally similar or identical in structure and/or function to any of the catheters described in (i) U.S. patent application Ser. No. 17/529,018, titled “CATHETERS HAVING SHAPED DISTAL PORTIONS, AND ASSOCIATED SYSTEMS AND METHODS,” and filed Nov. 17, 2021, (ii) U.S. patent application Ser. No. 17/529,064, titled “CATHETERS HAVING STEERABLE DISTAL PORTIONS, AND ASSOCIATED SYSTEMS AND METHODS,” and filed Nov. 17, 2021, (iii) U.S. patent application Ser. No. 18/159,507, titled “ASPIRATION CATHETERS HAVING GROOVED INNER SURFACE, AND ASSOCIATED SYSTEM AND METHODS,” and filed Jan. 25, 2023, and/or (iv) U.S. patent application Ser. No. 18/463,960, titled “CATHETERS HAVING MULTIPLE COIL LAYERS, AND ASSOCIATED SYSTEMS AND METHODS,” and filed Sep. 8, 2023, each of which is incorporated by reference herein in its entirety.

The reinfusion catheter 106 can be an elongate member (e.g., a sheath, a shaft) defining a reinfusion lumen 108 and configured to be inserted into and through the patient's vasculature and used to, for example, reinfuse filtered blood into the vasculature. The reinfusion catheter 106 can have a distal end portion 107 configured to be positioned within the vasculature, and can have a construction the same as or different than the aspiration catheter 102 described in detail above. In some embodiments, the reinfusion catheter 106 can be separate from the aspiration catheter 102 and can be inserted into the patient through a vascular access site different than a vascular access site of the aspiration catheter 102. In some such embodiments, the reinfusion catheter 106 can have a smaller size and corresponding inner diameter than the aspiration catheter 102.

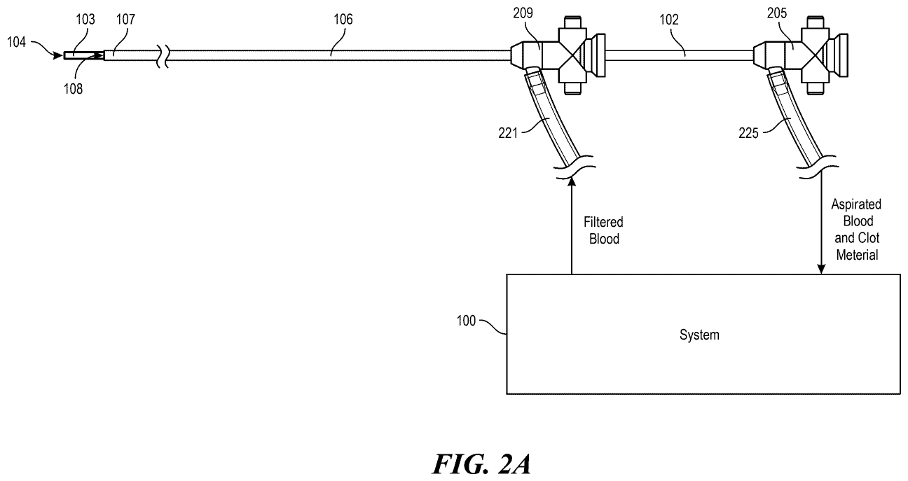

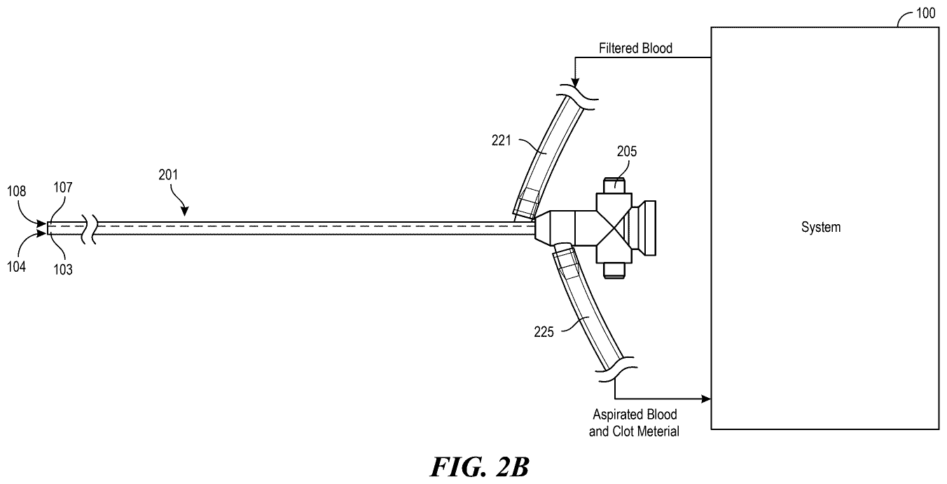

In other embodiments, the reinfusion catheter 106 can serve as introducer catheter (e.g., sheath) for the aspiration catheter 102. For example, FIG. 2A is a partially-schematic side view of the aspiration catheter 102 and the reinfusion catheter 106 of FIG. 1 with the reinfusion catheter 106 configured as an introducer in accordance with embodiments of the present technology. In the illustrated embodiment, a proximal end portion of the aspiration catheter 102 is coupled to an aspiration access valve 205 (e.g., a hemostasis valve) configured to selectively provide access to the aspiration lumen 104 of the aspiration catheter 102, and a proximal end portion of the reinfusion catheter 106 is coupled to a reinfusion access valve 209 (e.g., a hemostasis valve) configured to selectively provide access to the reinfusion lumen 108 of the reinfusion catheter 106. The aspiration and reinfusion access valves 205, 209 can be of the type disclosed in U.S. Pat. No. 11,559,382, titled “HEMOSTASIS VALVES AND METHODS OF USE,” and filed Aug. 30, 2018, which is incorporated herein by reference in its entirety. The aspiration catheter 102 can extend through the reinfusion access valve 209 and through the reinfusion lumen 108 of the aspiration catheter 102 such that, for example, the distal end portion 103 of the aspiration catheter 102 extends distally beyond the distal end portion 107 of the reinfusion catheter 106. In some embodiments, one or more clot treatment devices (e.g., mechanical thrombectomy devices) can be inserted through the aspiration access valve 105 during a clot treatment procedure using the system 100.

A first access conduit or tube 221 can fluidly couple the aspiration lumen 104 of the aspiration catheter 102 to the system 100 (e.g., to the collection chamber 110). A second access conduit or tube 225 can fluidly couple the reinfusion lumen 108 of the reinfusion catheter 106 to the system 100 (e.g., to the collection chamber 110). As described in further detail below, the system 100 can aspirate blood and clot material through the aspiration lumen 104 of the aspiration catheter 102, filter the blood from the clot material, and reinfuse/return the filtered blood through the reinfusion lumen 108 of the reinfusion catheter 106. In some aspects of the present technology, configuring the reinfusion catheter 106 as an introducer sheath/catheter for the aspiration catheter 102 can reduce the number of vascular access sites needed for a clot treatment procedure-improving procedure efficiency and reducing patient discomfort.

In further embodiments, the reinfusion catheter 106 and the aspiration catheter 102 can comprise different lumens of the same multi-lumen catheter. For example, FIG. 2B is a partially-schematic side view of a multi-lumen (e.g., dual-lumen) catheter 201 serving as both the aspiration catheter 102 and the reinfusion catheter 106 of FIG. 1 in accordance with embodiments of the present technology. In the illustrated embodiment, the aspiration lumen 104 extends at least partially parallel to the reinfusion lumen 108, and the distal end portion 103 of the aspiration lumen 104 terminates at the same location as the distal end portion 107 of the reinfusion lumen 108. In some embodiments, the aspiration lumen 104 has a greater cross-sectional dimension (e.g., area, diameter, radius) than a corresponding cross-sectional dimension of the reinfusion lumen 108. In some aspects of the present technology, the system 100 can aspirate blood and clot material and reinfuse filtered blood at different volumetric rates-enabling the aspiration lumen 104 to have a larger dimension than the reinfusion lumen 108 to, for example, maximize aspiration forces. In some embodiments, the multi-lumen catheter 201 can be of the type described in U.S. patent application Ser. No. 18/885,201, titled “MULTI-LUMEN ASPIRATION CATHETERS, AND ASSOCIATED SYSTEMS AND METHODS,” and filed Sep. 13, 2024, which is incorporated by reference herein in its entirety. In some embodiments, a proximal end portion of the multi-lumen catheter 201 is coupled to the access valve 205 configured to selectively provide access to the aspiration lumen 104.

The first access tube 221 can fluidly couple the aspiration lumen 104 of the aspiration catheter 102 to the system 100 (e.g., to the collection chamber 110). The second access tube 225 can fluidly couple the reinfusion lumen 108 of the reinfusion catheter 106 to the system 100 (e.g., to the collection chamber 110). As described in further detail below, the system 100 can aspirate blood and clot material through the aspiration lumen 104, filter the blood from the clot material, and reinfuse/return the filtered blood through the reinfusion lumen 108. In some aspects of the present technology, integrating the reinfusion catheter 106 and the aspiration catheter 102 can reduce the number of vascular access sites needed for a thrombectomy procedure as well as reduce the overall profile needed for the access site-improving procedure efficiency and reducing patient discomfort.

Referring to FIG. 1, in the illustrated embodiment the collection chamber 110 comprises a body or housing 118 having an upper portion 111 (e.g., a first portion) and a lower portion 112 (e.g., a second portion). A splashguard or lid 113 can be releasably/movably coupled to the housing 118, such as to the upper portion 111 thereof. In some embodiments, the lid 113 is pivotably coupled at one edge to the upper portion 111 of the housing 118. In other embodiments, the lid 113 is coupled to the housing 118 in a different manner (e.g., slidably coupled) or simply configured to rest on and potentially mate with (e.g., via a snap fit, friction fit) the upper portion 111 of the housing 118.

The housing 118 can define an interior or chamber 114 and one or more openings/ports extending through the housing 118 that provide fluid access to the chamber 114. For example, in the illustrated embodiment the collection chamber 110 includes an aspiration port 115 (e.g., an aspiration inlet), a vacuum port 116 (e.g., a vacuum outlet, a negative pressure port, a negative pressure outlet), and a positive pressure port 117 (e.g., a positive pressure inlet, an exhaust port, an exhaust inlet). In some embodiments, the aspiration port 115 is positioned near the upper portion 111 of the housing 118 and/or the lid 113. In the illustrated embodiment, the vacuum port 116 is similarly positioned near the upper portion 111 of the housing 118 while the positive pressure port 117 is below the vacuum port 116. In other embodiments, the aspiration port 115, the vacuum port 116, and/or the positive pressure port 117 (collectively ports “115-117”) can be positioned differently along the housing 118. For example, the vacuum port 116 and the positive pressure port 117 can extend through the housing 118 at the same vertical level, the vacuum port 116 and the positive pressure port 117 can be positioned on opposing sides of the housing 118, each of the ports 115-117 can be positioned on different sides of the housing 118, one or more of the ports 115-117 can extend through the lid 113, etc.

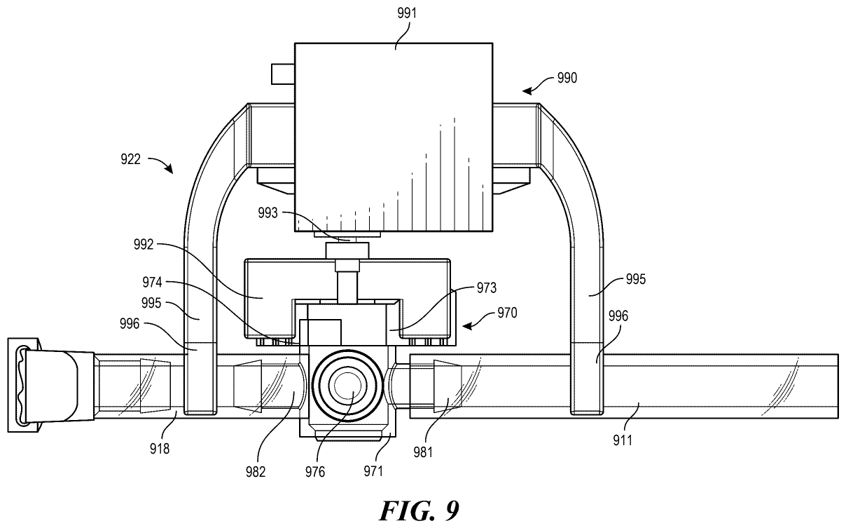

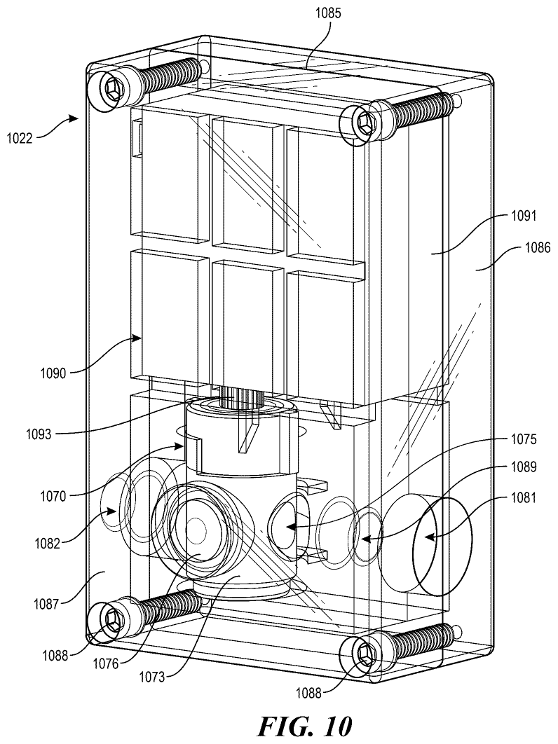

In the illustrated embodiment, the aspiration port 115 is fluidly coupled to an aspiration conduit or tube 121, which is fluidly coupled to the aspiration lumen 104 of the aspiration catheter 102. An aspiration valve 122 is positioned along the aspiration conduit 121 and is movable/actuatable (e.g., via the control system 180) to provide a fluid path therethrough from the aspiration lumen 104 of the aspiration catheter 102 to the chamber 114 of the collection chamber 110 via the aspiration port 115. That is, the aspiration valve 122 can be actuated to move between (i) an open position in which the aspiration catheter 102 is fluidly coupled to the collection chamber 110 (e.g., fluid is able to flow from the aspiration lumen 104 to the chamber 114) and (ii) a closed position in which the aspiration catheter 102 is fluidly decoupled from the collection chamber 110 (e.g., fluid is inhibited or even prevented from flowing from the aspiration lumen 104 to the chamber 114). Although described as a single conduit, the aspiration conduit 121 can comprise one or more discrete components fluidly coupled together, such as a first tube between a first (e.g., distal) side of the aspiration valve 122 and the aspiration catheter 102 and a second tube between a second (e.g., proximal) side of the aspiration valve 122 and the aspiration port 115. In some embodiments, the aspiration valve 122 is an automated (e.g., large bore) stopcock valve as described in detail below, for example, with reference to FIGS. 9 and 10. Likewise, the aspiration valve 122 can be a stopcock of any of the types described in U.S. patent application Ser. No. 18/182,966, titled “FLUID CONTROL DEVICES FOR CLOT TREATMENT SYSTEMS, AND ASSOCIATED SYSTEMS AND METHODS,” and filed Aug. 22, 2024, that can be controlled by the control system 180 to open and close. In other embodiments, the aspiration valve 122 can be a solenoid valve, a ball valve, a pinch valve, and/or the like that can be controlled by the control system 180 to selectively permit/inhibit fluid flow therethrough.

In some embodiments, the aspiration conduit 121 and the aspiration valve 122 each have a size and a corresponding inner diameter defining a lumen (e.g., bore) having a size that is the same as or greater than a size of the aspiration catheter 102 and a corresponding inner diameter defining the aspiration lumen 104. For example, the aspiration conduit 121 and the aspiration valve 122 can each have a size and corresponding inner diameter equal to or greater than about 16 French, about 18 French, about 20 French, about 22 French, about 22 French, about 24 French, and/or the like. As described in detail below, negative pressure generated in the chamber 114 can be applied to the aspiration catheter 102 when the aspiration valve 122 is opened to aspirate clot material and blood through the aspiration catheter 102, through the aspiration conduit 121, through the aspiration valve 122, through the aspiration port 115, and into the chamber 114.

In the illustrated embodiment, the vacuum port 116 of the collection chamber 110 is fluidly coupled to a vacuum conduit or tube 123, which is fluidly coupled to the pump assembly 140. Likewise, the positive pressure port 117 of the collection chamber 110 is fluidly coupled to a positive pressure conduit or tube 124, which is fluidly coupled to the pump assembly 140. As described in greater detail below, the pump assembly 140 can generate negative and positive pressure within the chamber 114 via the vacuum conduit 123 and the positive pressure conduit 124, respectively. In some embodiments, the vacuum conduit 123 and/or the positive pressure conduit 124 have a size and a corresponding inner diameter defining a lumen (e.g., bore) having a size that is the same as or smaller than a size of the aspiration conduit 121. For example, the vacuum conduit 123 and the positive pressure conduit 124 can have sizes and corresponding inner diameters that are smaller than the aspiration conduit 121.

The collection chamber 110 can further include a filter plate or filter tray 130 spanning laterally across the chamber 114 below the aspiration port 115, and a collection component 131 spanning laterally across the chamber 114 below the filter tray 130. In some embodiments, the collection component 131 slopes downward in a direction toward a central axis C of the collection chamber 110 and includes a lowermost receiving portion 132 configured to receive a filter 133 therein.

In the illustrated embodiment, the filter 133 is fluidly coupled to a reinfusion conduit or tube 125, which is fluidly coupled to the reinfusion lumen 108 of the reinfusion catheter 106. That is, the filter 133 comprises a port/outlet from the housing 118. A reinfusion valve 126 is positioned along the reinfusion conduit 125 and is movable/actuatable (e.g., via the control system 180) to provide a fluid path therethrough from the chamber 114 to the reinfusion lumen 108 of the reinfusion catheter 106 via the filter 133 and the reinfusion conduit 125. That is, the reinfusion valve 126 can be actuated to move between (i) an open position in which the reinfusion catheter 106 is fluidly coupled to the collection chamber 110 (e.g., fluid is able to flow from the chamber 114 to the reinfusion lumen 108) and (ii) a closed position in which the reinfusion catheter 106 is fluidly decoupled from the collection chamber 110 (e.g., fluid is inhibited or even prevented from flowing from the chamber 114 to the reinfusion lumen 108). Although described as a single conduit, the reinfusion conduit 125 can comprise one or more discrete components fluidly coupled together, such as a first tube between a first (e.g., proximal) side of the reinfusion valve 126 and the filter 133 and a second tube between a second (e.g., distal) side of the reinfusion valve 126 and reinfusion catheter 106. The reinfusion valve 126 can be a stopcock valve, a solenoid valve, a ball valve, a pinch valve, and/or the like that can be controlled by the control system 180 to selectively permit/inhibit fluid flow therethrough. In some embodiments, the reinfusion conduit 125 can have a size and a corresponding inner diameter defining a lumen (e.g., bore) having a size that is the same as or smaller than a size of the aspiration conduit 121. For example, the reinfusion conduit 125 can have a size and corresponding inner diameter that is smaller than the aspiration conduit 121 and the same as or similar to the vacuum conduit 123 and the positive pressure conduit 124.

In some embodiments, the filter tray 130 has a first porosity and the filter 133 has a second porosity less than the first porosity (e.g., the filter tray 130 has larger pores than pores of the filter 133 to permit larger particles to pass therethrough). For example, the filter tray 130 can have a micron (μm) rating equal to or greater than about 500 μm, about 600 μm, about 700 μm, about 800 μm, about 900 μm, about 1000 μm, about 1100 μm, about 2000 μm, about 3000 μm, and/or the like. The filter 133 can have a micron rating equal to or greater than about 10 μm, about 20 μm, about 30 μm, about 40 μm, about 50 μm, about 100 μm, about 200 μm, about 300 μm, about 400 μm, about 500 μm, and/or the like. In some embodiments, the filter 133 comprises multiple filter layers having different porosities. For example, the filter 133 can comprise a first filter layer having a first porosity and a second filter layer having a second porosity less than the first porosity. The first filter layer can be positioned radially outward relative to the second filter layer. In some embodiments, the first filter layer has a micron rating of about 200 μm and the second filter layer has a micron rating of about 40 μm. In some embodiments, the filter 133 comprises one or more filter layers arranged in a pleated arrangement about the central axis C and positioned to allow fluid (e.g., blood) to pass laterally therethrough from the receiving portion 132 of the collection component 131 to the reinfusion conduit 125. In some embodiments, the filter assembly can have some components generally similar or identical to, and can operate generally similarly or identically, to any of the filter devices described in U.S. Nonprovisional patent application Ser. No. 18/963,471, filed Nov. 27, 2024, titled “FILTERING DEVICES, SUCH AS FOR USE WITH CLOT TREATMENT SYSTEMS, AND ASSOCIATED SYSTEMS AND METHODS,” which is incorporated by reference herein in its entirety.

In operation, the collection chamber 110 can receive blood and clot material into the chamber 114 through the aspiration port 115 from the aspiration catheter 102 (e.g., via the aspiration conduit 121 when the aspiration valve 122 is opened). The blood and clot material can move (e.g., flow) downward toward/onto/through the filter tray 130 via gravity. The filter tray 130 can inhibit or even prevent larger portions of the aspirated material (e.g., larger portions of the clot material, coagulated blood) from flowing therethrough while permitting smaller portions of the aspirated material (e.g., blood, smaller portions of the clot material) to move therethrough downward toward/onto the collection component 131. Accordingly, the filter tray 130 provides a first filter stage that filters out large portions of the clot material. The collection component 131 can direct the first-stage filtered material toward the filter 133 positioned within the receiving portion 132. In some embodiments, as described in greater detail below, the pump assembly 140 can generate positive pressure within the chamber 114 via the positive pressure conduit 124 to drive the first-stage filtered material through the filter 133, through the reinfusion conduit 125, and through the reinfusion valve 126 when the reinfusion valve 126 is open. The filter 133 can provide a second filtering stage that filters out smaller portions of clot material while permitting blood to pass therethrough. Additionally or alternatively, the first-stage filtered material can flow through the filter 133 at least partially due to gravity. Accordingly, the collection chamber 110 is configured to receive aspirated blood and clot material from the aspiration catheter 102, filter the clot material from the blood, and permit the filtered blood to pass therefrom to the reinfusion catheter 106 via the reinfusion conduit 125. In some aspects of the present technology, the filter tray 130 can filter out large portions of the clot material that may otherwise clog or interfere with the operation of the filter 133.

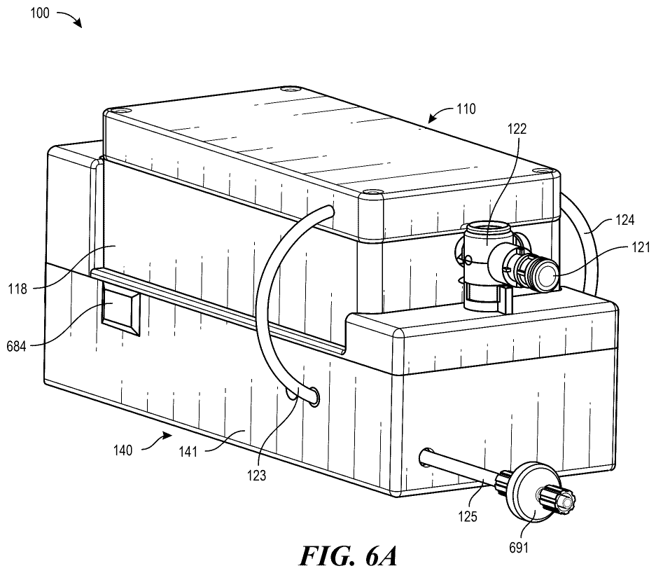

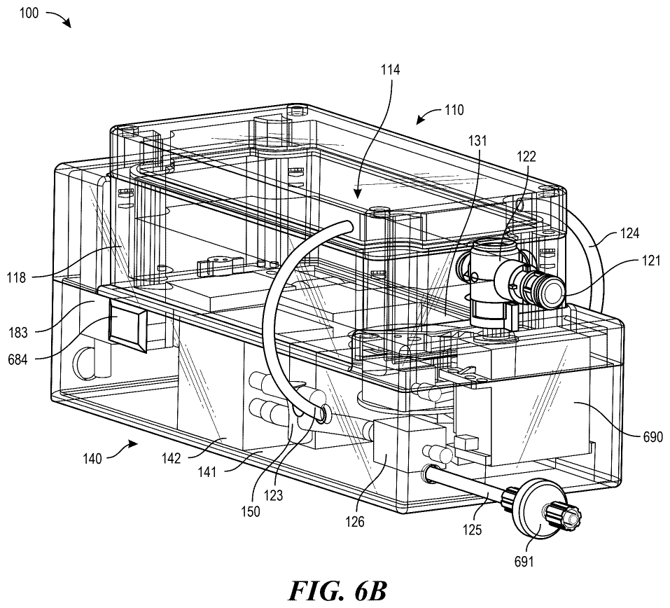

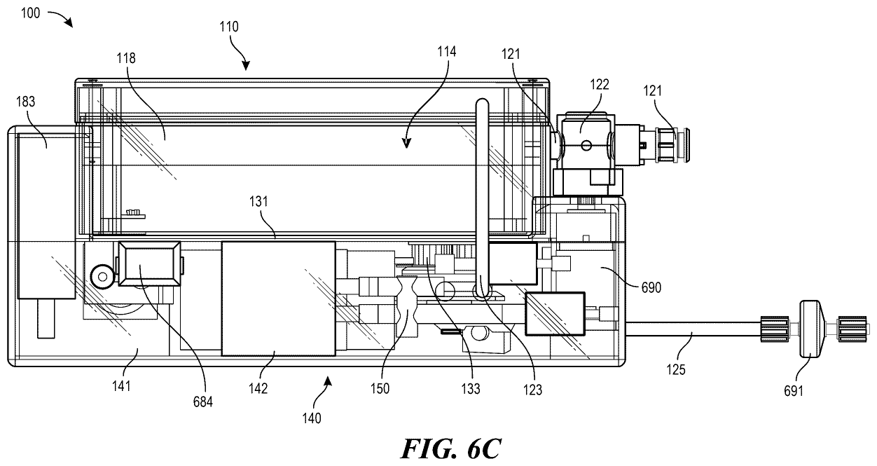

In some embodiments, the housing 118 and/or the lid 113 are at least partially transparent to allow a user of the system 100 (e.g., a surgeon and/or healthcare team member) to view clot material collected on the filter tray 130 and/or proximate to the filter 133. In the illustrated embodiment, the collection component 131 divides the chamber 114 into an upper chamber portion 119a and a lower chamber portion 119b. The upper chamber portion 119a is configured to receive the aspirated blood and clot material and to be subjected to negative and positive pressures generated by the pump assembly 140 during clot removal operations. In some embodiments, the upper chamber portion 119a can have a volume equal to or greater than about 100 cubic centimeters (cc), about 200 cc, about 300 cc, about 400 cc, about 500 cc, about 600 cc, about 1000 cc, greater than 1000 cc, and/or the like. In some embodiments, the lower chamber portion 119b is substantially sterile and/or isolated from the aspirated material and/or can remain at atmospheric pressure during aspiration and reinfusion operations of the system 100. In some embodiments, some or all of the components of the control system 180 and/or the pump assembly 140 are positioned/housed within the lower chamber portion 119b. In some embodiments, some or all of the components of the control system 180 and/or the pump assembly 140 are positioned/housed in the housing 141 of the pump assembly 140 as, for example, described in greater detail below with reference to FIGS. 6A-6C.

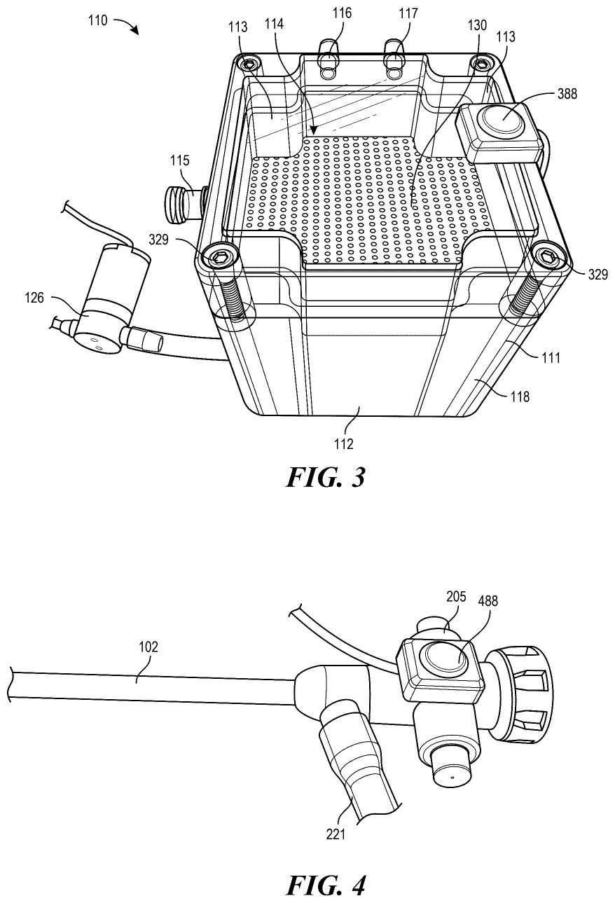

FIG. 3 is a perspective view of the collection chamber 110 in accordance with embodiments of the present technology. In the illustrated embodiment, the housing 118 has a rectilinear (e.g., rectangular, square) cross-sectional shape and the lid 113 is secured to the upper portion 111 of the housing 118 via one or more fasteners 329 (e.g., screws, rivets, and/or the like). The aspiration port 115 extends from/through a first side of the housing 118 at a vertical level above the filter tray 130. The aspiration port 115 can comprise, for example, a connector, such as a large bore (e.g., 24 French, 20 French, greater than 16 French) Toomey tip connector configured to be fluidly coupled to the aspiration conduit 121 (FIG. 1). In the illustrated embodiment, the vacuum port 116 and the positive pressure port 117 extend from/through a same side of the lid 113 different than a side of the aspiration port 115. In some embodiments, the vacuum port 116 and the positive pressure port 117 are positioned at a same vertical level above the filter tray 130. Similar to the aspiration port 115, the vacuum port 116 and the positive pressure port 117 can each comprise a connector, such as a Toomey tip connector or other fluid connector. In the illustrated embodiment, the reinfusion conduit 125 extends from below the lower portion 112 of the housing 118 in a same or generally similar direction as the aspiration port 115. In the illustrated embodiment, the reinfusion valve 126 comprises a solenoid valve. In the illustrated embodiment, the housing 118 and the lid 113 are at least partially transparent to, for example, permit visualization of clot material positioned on the filter tray 130 or below on the collection component 131 (FIG. 1; obscured in FIG. 3).

Referring again to FIG. 1, in the illustrated embodiment the pump assembly 140 includes a housing 141 enclosing a pump 142 having an inlet 143 and an outlet 144, a vacuum conduit or tubing assembly 145, a positive pressure conduit or tubing assembly 146, a vacuum valve 150, a positive pressure valve 151, a fluid (e.g., air) inlet valve 152, and a fluid (e.g., air) outlet valve 153. In some embodiments, each of the vacuum valve 150, the positive pressure valve 151, and the fluid inlet valve 152 (collectively “valves 150-152”) is an electromechanical valve configured to be controlled by the control system 180 to open and close (e.g., move between an open position and a closed position). For example, the valves 150-152 can be stopcock valves, solenoid valves, ball valves, and/or the like that can be controlled by the control system 180 to selectively permit/inhibit fluid flow therethrough. The valves 150-152 can be similar or identical. The fluid outlet valve 153 can be a check valve or other passive valve configured to (i) permit fluid flow (e.g., air flow) therethrough from the positive pressure conduit assembly 146 to the atmosphere or other outlet when a pressure in the positive pressure conduit assembly 146 is above a predetermined level and (ii) inhibit or even prevent fluid therethrough from the atmosphere or other outlet when the pressure in the positive pressure conduit assembly 146 is below the predetermined level.

In the illustrated embodiment, the vacuum valve 150 is movable/actuatable (e.g., via the control system 180) to provide a fluid path therethrough from the vacuum conduit 123 to the inlet 143 of the pump 142 via the vacuum conduit assembly 145. That is, the vacuum valve 150 can be actuated to move between (i) an open position in which the collection chamber 110 is fluidly coupled to the pump 142 (e.g., fluid is able to flow from the chamber 114 to and through the inlet 143 of the pump 142) and (ii) a closed position in which the collection chamber 110 is fluidly decoupled from the pump 142 (e.g., fluid is inhibited or even prevented flowing from the chamber 114 to the inlet 143 of the pump 142). The fluid inlet valve 152 can be movable/actuatable (e.g., via the control system 180) to provide a fluid path therethrough from the atmosphere or another fluid source to the inlet 143 of the pump 142 via the vacuum conduit assembly 145. That is, the fluid inlet valve 152 can be actuated to move between (i) an open position in which the atmosphere or other fluid source is fluidly coupled to the pump 142 (e.g., fluid is able to flow from the atmosphere or other source to and through the inlet 143 of the pump 142) and (ii) a closed position in which the atmosphere or other source is fluidly decoupled from the pump 142 (e.g., fluid is inhibited or even prevented flowing from the atmosphere or other source to the inlet 143 of the pump 142).

In the illustrated embodiment, the positive pressure valve 151 is movable/actuatable (e.g., via the control system 180) to provide a fluid path therethrough from the positive pressure conduit 124 to the outlet 144 of the pump 142 via the positive pressure conduit assembly 146. That is, the positive pressure valve 151 can be actuated to move between (i) an open position in which the collection chamber 110 is fluidly coupled to the pump 142 (e.g., fluid is able to flow from the outlet 144 of the pump 142 to the chamber 114) and (ii) a closed position in which the collection chamber 110 is fluidly decoupled from the pump 142 (e.g., fluid is inhibited or even prevented flowing from the outlet 144 of the pump 142 to the chamber 114).

The pump 142 can be a vacuum pump, such as a peristaltic pump, rotary pump, diaphragm pump, and/or the like, configured to intake fluid (e.g., air) via the inlet 143 and exhaust the fluid via the outlet 144. Accordingly, with the vacuum valve 150 open and the fluid inlet valve 152 closed, the pump 142 can operate to evacuate fluid (e.g., air) from the chamber 114 along the fluid path through the vacuum conduit 123, through the vacuum valve 150, through the vacuum conduit assembly 145, and to the inlet 143 of the pump 142 to generate vacuum pressure within the chamber 114. With the vacuum valve 150 closed and the fluid inlet valve 152 open, the pump 142 can operate to draw fluid (e.g., air) from the atmosphere or other source through the fluid inlet valve 152, through the vacuum conduit assembly 145, and to the inlet 143 of the pump 142.

The pump 142 can further operate to force the air or other fluid taken in through the inlet 143 out of the outlet 144 into the positive pressure conduit assembly 146. Accordingly, with the positive pressure valve 151 open, the pump 142 can drive the fluid into the chamber 114 along the fluid path from the outlet 144, through the positive pressure conduit assembly 146, through the positive pressure valve 151, and through the positive pressure conduit 124 to generate positive pressure within (e.g., pressurize) the chamber 114. In some embodiments, the fluid outlet valve 153 is configured to remain closed or at least partially closed when the positive pressure valve 151 is open and to open when the positive pressure valve 151 is closed. For example, the fluid outlet valve 153 can be pressure-sensitive to open and close based on a predetermined level of pressure within the positive pressure conduit assembly 146. Accordingly, when the positive pressure valve 151 is closed, the pump 142 can drive the fluid into the positive pressure conduit assembly 146 until the pressure exceeds the predetermined level and the fluid outlet valve 153 opens. With the fluid outlet valve 153 open, the pump 142 can drive the fluid through the fluid from the outlet 144, through the positive pressure conduit assembly 146, and out of the fluid outlet valve to the atmosphere or other source.

Accordingly, by utilizing both the fluid intake and fluid exhaust of the pump 142, the pump assembly 140 can operate to both (i) generate negative pressure within the chamber 114 for aspiration operations and (ii) generate positive pressure within the chamber 114 for reinfusion and filtering operations. In some aspects of the present technology, this can reduce the cost and complexity of the system 100 by, for example, not needing separate sources for generating vacuum and positive pressure.

In some embodiments, the system 100 further includes a first filter 147 positioned along the vacuum conduit 123 between the vacuum port 116 and the vacuum valve 150. The first filter 147 can be configured to trap/filter any liquids, bacteria, etc., that may be drawn from the chamber 114 toward the pump 142 when the vacuum valve 150 is open. Accordingly, the first filter 147 can inhibit or even prevent any clot material, blood, and/or other materials aspirated into the chamber 114 of the collection chamber 110 from being drawn into the pump assembly 140—ensuring, for example, that only air (e.g., filtered air) is evacuated from the chamber 114 by the pump assembly 140. In some embodiments, the system 100 further includes a second filter 148 positioned along the positive pressure conduit assembly 146 between the outlet 144 of the pump 142 and the positive pressure valve 151. The second filter 148 can be configured to filter the fluid (e.g., air) output from the outlet 144 of the pump. Accordingly, the second filter 148 can inhibit or even prevent any bacteria, impurities, contaminants, and/or the like from air drawn from, for example, the atmosphere through the fluid inlet valve 152 from being driven into the chamber 114 of the collection chamber 110 where they could potentially compromise blood therein for reinfusion.

In the illustrated embodiment, the pump assembly 140 is a self-contained unit that is not exposed to material aspirated from a patient such that the pump assembly 140 can be positioned within the sterile field during a clot removal procedure and reused in subsequent clot removal procedures (e.g., as capital equipment). Contrariwise, the collection chamber 110, the aspiration conduit 121, the aspiration catheter 102, the reinfusion conduit 125, the reinfusion catheter 106, etc., that are exposed to aspirated material can be positioned within the non-sterile field and thus disposable. In other embodiments, the pump assembly 140 can be configured for positioning in the non-sterile field and thus disposable as, for example, described in further detail below with reference to FIGS. 6A-6C. In some embodiments, the first filter 147 may be reusable and can be positioned within the housing 141. In some embodiments, the first and second filters 147, 148 can be replaceable after each clot removal procedure. Moreover, while the various components of the pump assembly 140 are shown enclosed within the housing 141, the housing 141 can be omitted and the various valves and conduits of the pump assembly 140 can be positioned elsewhere in the surgical field. For example, these components can be positioned within the lower chamber portion 119b of the collection chamber 110. Likewise, the pump 142 can comprise a vacuum pump that exists previously in a surgical operating room (e.g., as capital equipment).

The system 100 can optionally include one or more sensors communicatively coupled to the control system 180 and configured to detect information about conditions, operations, parameters, and/or the like of the system 100 during operation of the system 100 and communicate the information to the control system 180 to enable, for example, dynamic adjustments of the operation of the system 100 during a clot removal procedure. For example, the system 100 can include one or more of a flow rate sensor 160, a vacuum level sensor 161, a tilt sensor 162, a fluid level sensor 163, and a bubble sensor 164 (collectively “sensors 160-164”).

The flow rate sensor 160 can be positioned between the aspiration catheter 102 and the collection chamber 110, such as along the aspiration conduit 121, and configured to detect a fluid flow rate between the aspiration catheter 102 and the collection chamber 110. The control system 180 can utilize the detected flow rate to control operation of the aspiration valve 122. For example, if the flow rate is above a predetermined level, it can indicate that the aspiration catheter 102 is not engaged with any clot material and/or that there is no or little clot material within the aspiration lumen 104. Accordingly, the control system 180 can close the aspiration valve 122 in response to the detected flow rate being above the predetermined level to cease aspiration of the aspiration catheter 102 to reduce blood loss from a patient.

The vacuum level sensor 161 can be positioned within the chamber 114 and configured to detect a vacuum level within the chamber 114. The control system 180 can utilize the detected vacuum level to control operation of the vacuum valve 150. For example, once the vacuum level reaches a predetermined level, the control system 180 can close the vacuum valve 150 to stop evacuation of the chamber 114 and maintain a selected level of vacuum within the chamber 114. Likewise, if the vacuum level falls below a predetermined level, the control system 180 can open the vacuum valve 150 to further evacuate the chamber 114 via the pump assembly 140 until, for example, the vacuum level reaches a selected level.

The tilt sensor 162 can be positioned within the chamber 114 and configured to detect an angle/orientation/tilt of the collection chamber 110 (e.g., the housing 118). The tilt sensor 162 can be an inclinometer and/or the like that measures tilt based on, for example, gravity. The control system 180 can utilize the detected tilt to control operation of the vacuum valve 150, the positive pressure valve 151, the reinfusion valve 126, and/or other valves within the system 100. For example, if the control system 180 detects that the detected tilt is above a predetermined level, the control system 180 can (i) close the vacuum valve 150 to inhibit or even prevent any liquid from being sucked into the pump assembly 140 through the vacuum port 116 until the tilt is resolved (e.g., the detected tilt is below the predetermined level), (ii) close the positive pressure valve 151 to inhibit or even prevent any air from being driven through the filter 133 into the reinfusion conduit 125 until the tilt is resolved, (iii) close the reinfusion valve 126 to inhibit or even prevent any air from being driven through the reinfusion conduit 125 into the reinfusion catheter 106 until the tilt is resolved, and/or (iv) the like. In some embodiments, when the control system 180 determines that the tilt is above the predetermined level, the control system 180 can close each of the valves in the system 100 other than the fluid inlet valve 152 and/or cease operation of the pump 142.

The fluid level sensor 163 can be positioned within the collection chamber 110 and configured to detect a level of fluid (e.g., blood) within the chamber 114. The fluid level sensor 163 can comprise a pressure sensor, a capacitance sensor, and/or the like. The control system 180 can utilize the detected fluid level to control operation of the reinfusion valve 126 and/or the positive pressure valve 151. For example, the control system 180 can maintain the reinfusion valve 126 and the positive pressure valve 151 in open positions during reinfusion of filtered blood into the reinfusion catheter 106 until the detected fluid level reaches or falls below a predetermined level, upon which, the control system 180 can close the reinfusion valve 126 and/or the positive pressure valve 151 to stop filtered blood reinfusion to thereby inhibit any air from being driven into the reinfusion catheter 106.

The bubble sensor 164 can be positioned between the reinfusion catheter 106 and the collection chamber 110, such as along the reinfusion conduit 125 between the reinfusion valve 126 and the filter 133, and can be configured to detect the presence of bubbles in a fluid (e.g., filtered blood) moving between the reinfusion catheter 106 and the collection chamber 110. The control system 180 can control operation of the reinfusion valve 126 based on the detected presence of bubbles or lack thereof. For example, if bubbles are detected by the bubble sensor 164, the control system 180 can close the reinfusion valve 126 to thereby inhibit the bubbles (e.g., air bubbles) from being driven into the reinfusion catheter 106. The reinfusion conduit 125 can then be deaired (e.g., by a user) before continued operation of the system 100.

Referring again to FIG. 1, the control system 180 can include a non-transitory computer-readable medium 181, a processor 182, a power source 183, one or more user controls 184, and a display 185. The computer-readable medium 181 can store instructions that, when executed by the processor 182, carry out the functions attributed to the control system 180 as described herein. Although not required, aspects and embodiments of the present technology can be described in the general context of computer-executable instructions, such as routines executed by a general-purpose computer, e.g., a server or personal computer. Those skilled in the relevant art will appreciate that the present technology can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers, and the like. The present technology can be embodied in a special-purpose computer or data processor that is specifically programmed, configured, or constructed to perform one or more of the computer-executable instructions explained in detail below. Indeed, the term “computer” (and like terms), as used generally herein, refers to any of the above devices, as well as any data processor or any device capable of communicating with a network, including consumer electronic goods such as game devices, cameras, or other electronic devices having a processor and other components, e.g., network communication circuitry.

The present technology can also be practiced in distributed computing environments, where tasks or modules are performed by remote processing devices, which are linked through a communications network, such as a Local Area Network (“LAN”), Wide Area Network (“WAN”), or the Internet. In a distributed computing environment, program modules or sub-routines can be located in both local and remote memory storage devices. Aspects of the present technology described below can be stored or distributed on computer-readable media, including magnetic and optically readable and removable computer discs, stored as in chips (e.g., EEPROM or flash memory chips). Alternatively, aspects of the present technology can be distributed electronically over the Internet or over other networks (including wireless networks). Those skilled in the relevant art will recognize that portions of the present technology can reside on a server computer, while corresponding portions reside on a client computer. Data structures and transmission of data particular to aspects of the present technology are also encompassed within the scope of the present technology.

The power source 183 can provide power to the processor 182, to the various valves (e.g., the aspiration valve 122, the reinfusion valve 126, the vacuum valve 150, the positive pressure valve 151, the fluid inlet valve 152, etc.) to operate (e.g., open and close) the valves, to the sensors 160-164, and/or to other electrical components of the system 100. The power source 186 can comprise one or more batteries, a connection to a source of electrical power in a hospital (e.g., a wall plug and/or AC power source within an operating room), and/or the like. As described above, some or all of the non-transitory computer-readable medium 181, the processor 182, and the power source 183 can be located at least partially within the lower chamber portion 119b of the collection chamber 110 and/or within the housing 141 of the pump assembly 140.

The user controls 184 can comprise one or more buttons, sliders, touchscreen elements, and/or the like that can be actuated, triggered, selected, and/or the like by a user of the system 100 to control one or more operations of the system 100. The user controls 184 can be utilized by the user to start/stop some or all of the aspiration, filtering, and/or reinfusion operations described in detail herein. The user controls 184 can be operably coupled to the processor 182 via a wired or wireless connection. In some embodiments, the user controls 184 comprise one or more buttons, switches, actuators, and/or the like positioned on the aspiration catheter 102 (e.g., on a proximal portion thereof), on the reinfusion catheter 106 (e.g., on a proximal portion thereof), on the collection chamber 110 (e.g., on an external surface of the housing 118), on the pump assembly 140 (e.g., on an external surface of the housing 141) and/or elsewhere in/on the system 100. The user controls 184 can be actuatable to cause the system 100 to start/stop an aspiration cycle/sequence and/or start/stop a reinfusion cycle/sequence.

For example, referring to FIG. 3, the one or more user controls can comprise a button 388 positioned on/coupled to the collection chamber 110, such as positioned on the lid 113. The button 388 can be actuatable by a user to, for example, cause the control system 180 (FIG. 1) to initiate one or more filtered blood reinfusion operations. As another example, FIG. 4 is a perspective view of a proximal portion of the aspiration catheter 102 of FIG. 1 in accordance with embodiments of the present technology. In the illustrated embodiment, the aspiration catheter 102 has a proximal end portion coupled to the aspiration access valve 205 (e.g., a hemostasis valve) configured to selectively provide access to the aspiration lumen 104 (FIG. 1) of the aspiration catheter 102, and the aspiration lumen 104 is fluidly coupled to the collection chamber 110 (FIG. 1) via the first access tube 221. In the illustrated embodiment, the one or more user controls comprise a button 488 positioned on/coupled to the access valve 205. The button 488 can be actuatable by a user to, for example, cause the control system 180 (FIG. 1) to initiate one or more aspiration operations. In some aspects of the present technology, positioning the button 488 on and/or proximate to the aspiration catheter 102 can enable a user to maintain their hand(s) on the aspiration catheter 102 while simultaneously controlling aspiration via the aspiration catheter 102. Referring to FIG. 1, the user controls 184 can comprise any number of user-controlled actuators positioned within the system 100 to control various aspiration, reinfusion, and/or other operations of the system 100 described herein.

The display 185 can be coupled to and/or otherwise visible within the system 100 and configured to display information about the system 100 and/or provide for user input thereto (e.g., as a touchscreen). For example, the display 185 can display information related to (i) an open or closed state of any of the valves 122, 126, and 150-152, (ii) an operational state of the pump 142, (iii) a sensor reading of any of the sensors 160-164, and/or (iv) the like. In some embodiments, the display 185 is a touchscreen display, and the user controls 184 can be integrated with/embedded in the display 185. For example, the display 185 can enable a user to select a desired level of vacuum pressure within the chamber 114 (e.g., as indicated by the vacuum level sensor 161), a desired opening time of the aspiration valve 122 for a corresponding vacuum aspiration pulse/volume through the aspiration catheter 102, a desired opening time of the positive pressure valve 151 and/or the reinfusion valve 126 for a corresponding filtered blood reinfusion time/volume through the reinfusion catheter 106, etc. In some embodiments, the display 185 and/or the user controls 184 are configured to permit a user of the system 100 to precisely select (i) a generated vacuum pressure within the chamber 114 of the collection chamber 110, (ii) a specific volume of the vacuum pressure applied to the aspiration catheter 102 via opening of the aspiration valve 122 (e.g., 30 cc, 60 cc, 100 cc, 120 cc, etc.), (iii) a specific volume of filtered blood reinfused into the patient via the reinfusion catheter 106 by opening of the positive pressure valve 151 and the reinfusion valve 126, and/or (iv) the like.

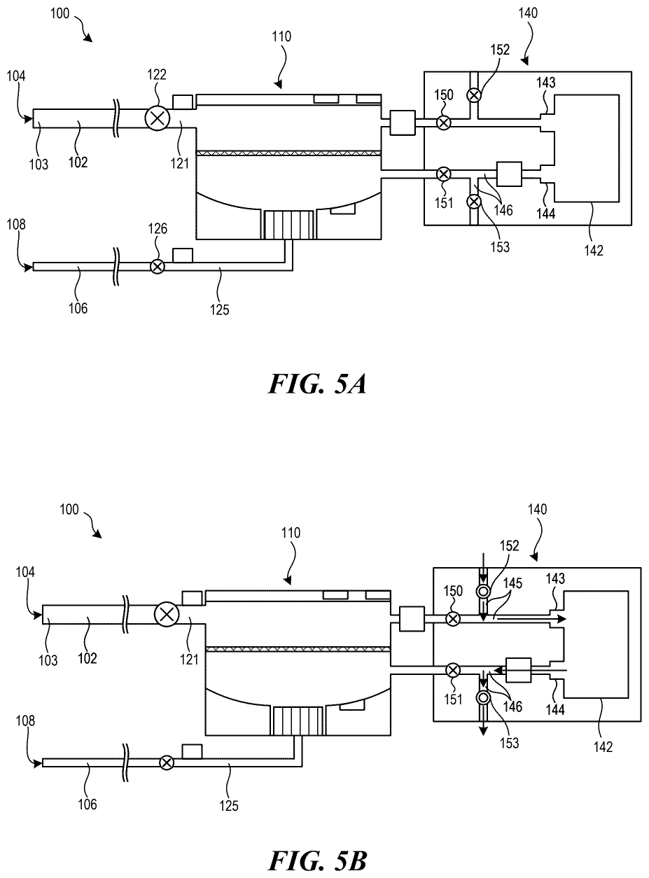

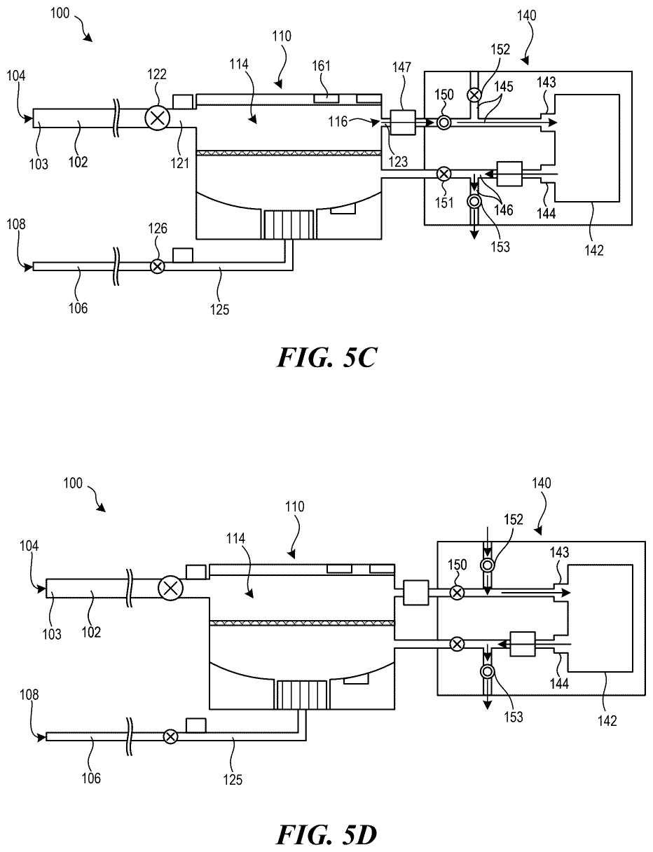

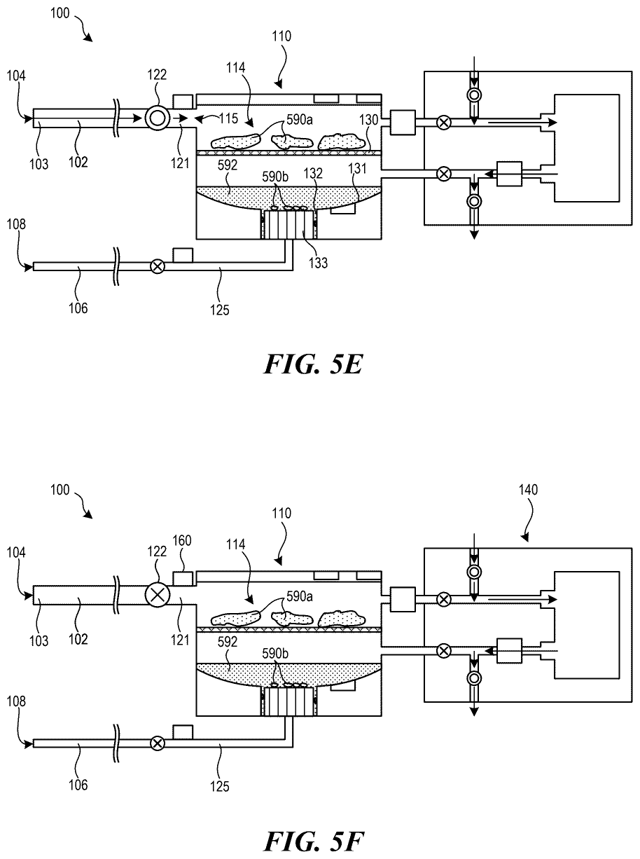

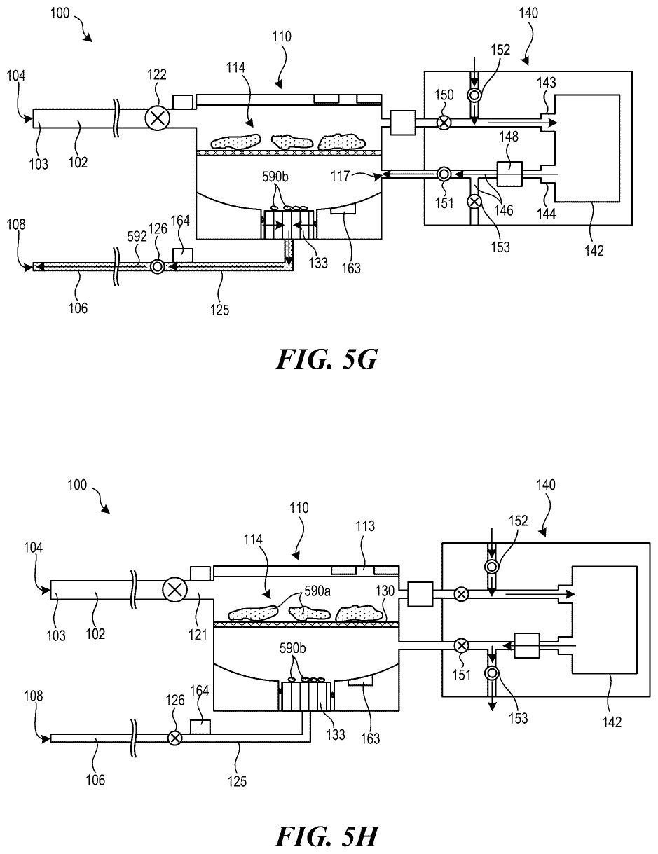

Referring to FIG. 1, the control system 180 can control the various valves (e.g., the aspiration valve 122, the reinfusion valve 126, the vacuum valve 150, the positive pressure valve 151, the fluid inlet valve 152, etc.), the pump 142, and/or other components of the system 100 in various sequences/cycles to perform a myriad of aspiration, blood filtering, and filtered blood reinfusion operations during a clot removal procedure (e.g., thrombectomy procedure) carried out on a patient. Some such operations are described in detail below with reference to FIGS. 5A-5H. However, one of ordinary skill in the art will appreciate that the various operations may be combined and/or modified.

FIGS. 5A-5H are partially-schematic side cross-sectional views of the clot treatment system 100 of FIG. 1 illustrating a method or sequence performed by the system 100 of aspirating clot material and blood from a patient, filtering the blood from the clot material, and reinfusing the filtered blood into the patient in accordance with embodiments of the present technology. The control system 180 is not shown in FIGS. 5A-5H for clarity. Referring to FIGS. 5A-5H, the distal end portion 103 of the aspiration catheter 102 can be positioned proximate to clot material (not shown) within the vasculature of a patient (not shown), such as proximal to, within, and/or distal to the clot material within the vasculature, and the aspiration conduit 121 of can be fluidly coupled to the aspiration lumen 104 of the aspiration catheter 102. In some embodiments, the clot material comprises a pulmonary embolism within a pulmonary artery of the patient, a deep vein thrombosis (DVT) within a peripheral vein of the patient, and/or the like. The reinfusion catheter 106 can likewise be positioned within the vasculature of the patient through the same or a different access site as the aspiration catheter, as described in detail above with reference to FIGS. 1-2B, and the reinfusion conduit 125 can be fluidly coupled to the reinfusion lumen 108 of the reinfusion catheter 106.

Referring to FIG. 5A, the system 100 can initially be in an off state (e.g., a non-powered state) in which (i) the pump 142 does not operate to move fluid (e.g., air) from the inlet 143 to the outlet 144 and (ii) each of the aspiration valve 122, the reinfusion valve 126, and the valves 150-152 of the pump assembly 140 is in a closed position (as indicated by an “X” on each valve), and (iii) the air outlet valve 153 is in a closed position (as indicated by an “X” on the air outlet valve 153). As described in detail above, the control system 180 can control the aspiration valve 122, the reinfusion valve 126, and the valves 150-152 to close, while the air outlet valve 153 can close passively in response to the pressure within the positive pressure conduit assembly 146 being below a predetermined level (e.g., because the pump 142 does not pressurize the positive pressure conduit assembly 146 when it is off).

Referring to FIG. 5B, the control system 180 (FIG. 1) can then move the system 100 to a first standby state by opening the fluid inlet valve 152 (as indicated by an “O” on the fluid inlet valve 152) and turning on the pump 142. As indicated by arrows, the pump 142 then operates to (i) draw fluid (e.g., air) through the fluid inlet valve 152 from the atmosphere or other source, through the vacuum conduit assembly 145, and through the inlet 143 and (ii) drive the fluid through the outlet 144, through the positive pressure conduit assembly 146, through the fluid outlet valve 153, and to the atmosphere or other source. The fluid outlet valve 153 can open automatically in response to the pump 142 generating positive pressure in the positive pressure conduit assembly 146 that is above the predetermined level. In the illustrated first standby state, the collection chamber 110 remains fluidly isolated from the pump assembly 140 via the closure of the vacuum valve 150 and the positive pressure valve 151. In some embodiments, one or more of the user controls 184 (FIG. 1) can be actuated to move the system 100 to the first standby state.

Referring to FIG. 5C, the control system 180 (FIG. 1) can then move the system 100 to a vacuum generation state by closing the fluid inlet valve 152 and opening the vacuum valve 150. As indicated by arrows, the pump 142 then operates to (i) draw fluid (e.g., air) from the chamber 114 of the collection chamber 110, through the vacuum port 116, through the vacuum conduit 123, through the first filter 147, through the vacuum valve 150, through the vacuum conduit assembly 145, and into the inlet 143 and (ii) drive the fluid through the outlet 144, through the positive pressure conduit assembly 146, through the fluid outlet valve 153, and to the atmosphere or other source. The fluid outlet valve 153 opens automatically in response to the pump 142 generating positive pressure in the positive pressure conduit assembly 146 that is above the predetermined level. Because each of the aspiration valve 122, the reinfusion valve 126, and the positive pressure valve 151 are closed, vacuum is charged within the chamber 114 (e.g., a negative pressure is maintained) before the chamber 114 is fluidly connected to the aspiration lumen 104 of the aspiration catheter 102. In some embodiments, the aspiration valve 122 can be opened when the vacuum valve 150 is opened such that vacuum is applied directly to the aspiration lumen 104 of the aspiration catheter 102. In some embodiments, one or more of the user controls 184 (FIG. 1) can be actuated to move the system 100 to the vacuum generation state. In some embodiments, the system 100 is configured to maintain the vacuum generation state until a selected level of vacuum is generated within the chamber 114, such as a vacuum of between about 60-600 cc, between about 60-150 cc, between about 60-120 cc, about 30 cc, about 60 cc, about 100 cc, about 200 cc, about 300 cc, about 400 cc, about 500 cc, about 600 cc, greater than about 600 cc, and/or the like. The vacuum level sensor 161 can provide an indication of the level of the vacuum within the chamber 114.

Referring to FIG. 5D, once the selected level of vacuum is generated within the chamber 114, the control system 180 can move the system 100 to a vacuum-charged state by opening the fluid inlet valve 152 and closing the vacuum valve 150. In the vacuum-charged state, the vacuum is maintained within the chamber 114, and the pump 142 again operates to drive fluid along the fluid path from the fluid inlet valve 152 to the fluid outlet valve 153. In some embodiments, the control system 180 can turn off the pump 142 in the vacuum-charged state.

Referring to FIGS. 5C and 5D, the control system 180 can control the amount of vacuum generated (e.g., charged) within the chamber 114 by controlling a duration that the vacuum valve 150 remains open (FIG. 5C) before closing the vacuum valve 150 (FIG. 5D). The duration can be preselected to correspond to a desired level of vacuum and/or can be controlled manually by a user (e.g., via one or more of the user controls 184 of FIG. 1) to generate the desired level of vacuum. For example, the one or more user controls 184 (FIG. 1) can comprise a button, switch, slider, touchscreen element, etc., that can be actuated by the user to cause the control system 180 to open the vacuum valve 150 for a preselected duration. In some such embodiments, the user controls 184 can further include another button, switch, slider, touchscreen element, etc., that is adjustable to adjust/change the preselected duration to allow for user selection of the amount of vacuum generated in the chamber 114 (e.g., 60 cc, 120 cc, 200 cc, etc.). That is, the user controls 184 can include one control for opening the vacuum valve 150 to generate vacuum within the chamber 114 and the same or another control for controlling the duration that the vacuum valve 150 remains open and thus the amount of vacuum generated within the chamber 114. In some embodiments, the user controls 184 include an actuation mechanism (e.g., button) that can be activated (e.g., pressed) by the user and that maintains the vacuum valve 150 in the open position until the user deactivates (e.g., releases) the actuation mechanism.

Referring to FIG. 5E, the control system 180 can then move the system 100 to an aspiration state by opening the aspiration valve 122 to fluidly connect the chamber 114 of the collection chamber 110 to the aspiration lumen 104 of the aspiration catheter 102 via the aspiration conduit 121. Accordingly, at least a portion of the vacuum stored in the chamber 114 is applied to the aspiration lumen 104 of the aspiration catheter 102 as indicated by arrows to aspirate clot material 590 (identified as clot material 590a and 590b) and blood 592 through the aspiration lumen 104 of the aspiration catheter 102, through the aspiration valve 122, through the aspiration conduit 121, through the aspiration port 115, and into the chamber 114. In some embodiments, opening the aspiration valve 122 instantaneously or nearly instantaneously applies at least a portion of the stored vacuum pressure to the aspiration lumen 104 of the aspiration catheter 102, thereby generating a suction pulse throughout the aspiration lumen 104 that can aspirate the clot material 590 and the blood 592 into and through the aspiration lumen 104. In some aspects of the present technology, such aspiration via stored/charged vacuum pressure can have a multitude of benefits as described in, for example, U.S. Pat. No. 11,559,382, titled “SYSTEM FOR TREATING EMBOLISM AND ASSOCIATED DEVICES AND METHODS,” and filed Aug. 8, 2019, which is incorporated herein by reference in its entirety.

The clot material 590 and the blood 592 are received through the aspiration port 115 of the collection chamber 110, and gravity causes the clot material 590 and the blood 592 to move downward through the chamber 114 toward the filter tray 130 and the collection component 131. As described in detail above with reference to FIG. 1, the filter tray 130 can filter larger portions 590a of the clot material 590 while allowing the blood 592 and smaller portions 590b of the clot material 590 to pass therethrough. The blood 592 and the smaller portions 590b of the clot material 590a can collect on the collection component 131 and/or in the receiving portion 132 thereof around the filter 133.

Referring to FIG. 5F, once a selected level of vacuum is applied to the aspiration catheter 102 (e.g., a selected aspiration volume or pulse), the control system 180 can move the system 100 to a collected state by closing the aspiration valve 122. In the collected state, the chamber 114 is fluidly isolated from the aspiration catheter 102, the reinfusion catheter 106, and the pump assembly 140.

Referring to FIGS. 5E and 5F, the control system 180 can control the amount of vacuum applied to the aspiration catheter 102 by controlling a duration that the aspiration valve 122 remains open (FIG. 5E) before closing the aspiration valve 122 (FIG. 5F). The duration can be preselected to correspond to a desired level of vacuum/aspiration and/or can be controlled manually by a user (e.g., via one or more of the user controls 184 of FIG. 1) to apply the desired level of vacuum. For example, the one or more user controls 184 (FIG. 1) can comprise a button, switch, slider, touchscreen element, etc., that can be actuated by the user to cause the control system 180 to open the aspiration valve 122 for a preselected duration. More specifically, referring to FIG. 4, such a user control can comprise the button 488 positioned on the access valve 205 of the aspiration catheter 102. Referring again to FIGS. 5E and 5F, in some such embodiments the user controls 184 can further include another button, switch, slider, touchscreen element, etc., that is adjustable to adjust/change the preselected duration to allow for user selection of the amount of vacuum applied from the chamber 114 to the aspiration catheter 102 (e.g., 20 cc, 30 cc, 40 cc, 50 cc, 60 cc, 120 cc, 200 cc, etc.). That is, the user controls 184 can include one control for opening the aspiration valve 122 to apply the vacuum within the chamber 114 to the aspiration catheter 102 and the same or another control for controlling the duration that the aspiration valve 122 remains open and thus the amount of vacuum applied to the aspiration catheter 102. In some embodiments, the user controls 184 include an actuation mechanism (e.g., button) that can be activated (e.g., pressed) by the user and that maintains the aspiration valve 122 in the open position until the user deactivates (e.g., releases) the actuation mechanism. For example, referring additionally to FIG. 4, the control system 180 can open the aspiration valve 122 for as long as the user holds down the button 488—thereby allowing for direct user control of the aspiration duration and volume.

Accordingly, the system 100 is configured to apply a selected volume of vacuum to the aspiration catheter 102 to enable precise and targeted clot aspiration with minimal blood loss. For example, if the chamber 114 is charged with 400 cc of vacuum pressure in the vacuum-charged state (FIG. 5D), the aspiration valve 122 can be opened and closed as shown in FIGS. 5E and 5F to apply a vacuum aspiration pulse to the aspiration catheter 102 of any portion of the 400 cc, such as 20 cc, 30 cc, 60 cc, 120 cc, 200 cc, 400 cc, etc. Accordingly, the system 100 can apply multiple discrete pulses/bursts of vacuum pressure to the aspiration catheter to aspirate the clot material 590 and the blood 592 into the chamber 114. Each pulse can aspirate additional clot material 590 and blood 592 into the chamber 114. Likewise, the entire vacuum charged in the chamber 114 can be applied to the aspiration catheter 102 in a single pulse. In some embodiments, the flow rate sensor 160 can sense a flow rate of the clot material 590 and/or the blood 592 through the aspiration conduit 121 and, if the flow rate is above a predetermined level (e.g., indicating that there is little or none of the clot material 590 moving through the system 100), the control system 180 can automatically close the aspiration valve 122 to minimize the amount of the blood 592 aspirated into the chamber 114.

In some embodiments, the aspiration conduit 121 is relatively short such that the collection chamber 110 is positioned close to the aspiration catheter 102. This can minimize the length and volume of the flow path through the aspiration catheter 102 to the chamber 114. In some aspects of the present technology, this can increase the flow rate of the clot material 590 and the blood 592 through the aspiration catheter 102 and into the chamber 114 as compared to clot treatment systems in which the flow path between an aspiration container and vacuum source is relatively long. Such increased flow rates are expected to increase the ability of the system 100 to ingest the clot material 590—even where the clot material 590 comprises large, adherent, fibrous, and/or otherwise difficult to ingest clot material.

Referring to FIG. 5G, after at least some of the clot material 590 and the blood 592 are aspirated into the chamber 114 as shown in FIGS. 5E and 5F, the control system 180 can move the system 100 to a reinfusion state by opening the positive pressure valve 151 and the reinfusion valve 126. As indicated by arrows, the pump 142 then operates to drive the fluid drawn into the inlet 143 through the fluid inlet valve 152 out of the outlet 144, through the second filter 148, through the positive pressure conduit assembly 146, through the positive pressure valve 151, through the positive pressure port 117, and into the chamber 114. The fluid outlet valve 153 can at least partially close automatically in response to the positive pressure valve 151 being opened such that the pressure in the positive pressure conduit assembly 146 is below the predetermined level. Accordingly, the pump 142 generates positive pressure within (e.g., pressurizes) the chamber 114. Because each of the aspiration valve 122 and the vacuum valve 150 are closed, and the reinfusion valve 126 is open, the positive pressure generated by the pump 142 acts to drive/force the blood 592 and the smaller portions 590b of the clot material 590 toward the filter 133 and the reinfusion conduit 125. As described in detail above with reference to FIG. 1, the filter 133 filters out the smaller portions 590b of the clot material 590 from the blood 592 such that the blood 592 is suitable for reinfusion into the patient. As indicated by arrows, the positive pressure drives the blood 592 through the filter 133, through the reinfusion conduit 125, through the reinfusion valve 126, and at least partially through the reinfusion lumen 108 of the reinfusion catheter 106. At least some of the filtered blood 592 can exit the reinfusion lumen 108 into the vasculature of the patient.

Referring to FIG. 5H, once a selected volume of the filtered blood 592 (FIG. 5G) is reinfused through the reinfusion catheter 106, the control system 180 can move the system 100 to a second standby state by (i) closing the positive pressure valve 151 to fluidly disconnect the chamber 114 form the pump assembly 140 to cease pressurization of the chamber 114 and/or (ii) closing the reinfusion valve 126 to fluidly disconnect the reinfusion lumen 108 of the reinfusion catheter 106 from the chamber 114. In the illustrated embodiment, both the positive pressure valve 151 and the reinfusion valve 126 are closed such that second standby state is identical to the first standby state shown in FIG. 5B, except for the presence of the clot material 590 within the chamber 114. In the second standby state, the chamber 114 is fluidly isolated from the aspiration catheter 102, the reinfusion catheter 106, and the pump assembly 140. Likewise, the pump assembly 140 again directs fluid through the fluid inlet valve 152, through the pump 142, and out of the fluid outlet valve 153.

Referring to FIGS. 5G and 5H, the control system 180 can control the amount of the blood 592 reinfused through the reinfusion catheter 106 by controlling a duration that the positive pressure valve 151 and the reinfusion valve 126 remain open (FIG. 5G) before closing the positive pressure valve 151 and/or the reinfusion valve 126 (FIG. 5F). The duration can be preselected to correspond to a desired volume of the blood 592 to be reinfused and/or can be controlled manually by a user (e.g., via one or more of the user controls 184 of FIG. 1) to apply the positive pressure from the pump 142 to reinfuse the blood 592. For example, the one or more user controls 184 (FIG. 1) can comprise a button, switch, slider, touchscreen element, etc., that can be actuated by the user to cause the control system 180 to open the positive pressure valve 151 and the reinfusion valve 126 for a preselected duration. More specifically, referring to FIG. 3, such a user control can comprise the button 388 positioned on the collection chamber 110. Referring again to FIGS. 5G and 5H, in some such embodiments the user controls 184 can further include another button, switch, slider, touchscreen element, etc., that is adjustable to adjust/change the preselected duration to allow for user selection of the amount of the blood 592 reinfused through the reinfusion catheter 106 (e.g., 20 cc, 30 cc, 40 cc, 50 cc, 60 cc, 120 cc, 200 cc, etc.). That is, the user controls 184 can include one control for opening the positive pressure valve 151 and the reinfusion valve 126 to initiate reinfusion of the blood 592 through the reinfusion catheter 106 and the same or another control for controlling the duration that the positive pressure valve 151 and/or the reinfusion valve 126 remain open and thus the amount of the blood 592 reinfused through the reinfusion catheter 106. In some embodiments, the user controls 184 include an actuation mechanism (e.g., button) that can be activated (e.g., pressed) by the user and that maintains the positive pressure valve 151 and the reinfusion valve 126 in the open positions until the user deactivates (e.g., releases, depresses) the actuation mechanism. For example, referring additionally to FIG. 3, the control system 180 can open the positive pressure valve 151 and the reinfusion valve 126 for as long as the user holds down the button 388.

Accordingly, the system 100 is configured to generate positive pressure within the chamber 114 to enable precise and targeted filtered blood reinfusion to the patient. For example, if the chamber 114 is filled with 400 cc of the blood 592 in the collected state (FIG. 5F), the positive pressure valve 151 and the reinfusion valve 126 can be opened and closed as shown in FIGS. 5G and 5H to generate positive pressure within the chamber 114 to drive all or any portion of the 400 cc, such as 20 cc, 30 cc, 60 cc, 120 cc, 200 cc, 400 cc, etc., through the reinfusion catheter 106 into the vasculature of the patient Accordingly, the system 100 can apply multiple discrete pulses/bursts of positive pressure to the chamber 114 to drive the blood 592 through the filter 133 and into the reinfusion catheter 106. In some embodiments, the fluid level sensor 163 can sense a level of the blood 592 within the chamber 114 and, if the level is at or below a predetermined level, the control system 180 (FIG. 1) can close the positive pressure valve 151 and/or the reinfusion valve 126 to cease reinfusion of the blood 592 through the reinfusion catheter 106 to, for example, thereby inhibit any air from being driven into the reinfusion catheter 106. Similarly, if bubbles are detected by the bubble sensor 164, the control system 180 can close the reinfusion valve 126 to thereby inhibit the bubbles (e.g., air bubbles) from being driven into the reinfusion catheter 106.

Referring again to FIG. 5H, when the system 100 is in the second standby state, the control system 180 (FIG. 1) can control the system 100 to perform further vacuum generation, clot and blood aspiration, and filtered blood reinfusion operations as shown in FIGS. 5C-5H. Additionally, a user can open the lid 113 to provide access to the chamber 114 for removal of the clot material 590a, for cleaning of the filter tray 130, etc. Moreover, a flushing fluid can be introduced into the system 100 for flushing of the filter 133, the aspiration conduit 121, the aspiration catheter 102, the reinfusion conduit 125, the reinfusion catheter 106, and/or other components of the system 100. The pump assembly 140 can be controlled as detailed above to generate negative and/or positive pressure within the collection chamber 110 to drive the flushing fluid through the system 100.

Accordingly, referring to FIGS. 1 and 5A-5H, the control system 180 can control the various valves in various sequences to perform a myriad of aspiration, filtered blood reinfusion, and system flushing operations during a clot removal procedure (e.g., thrombectomy procedure) carried out on a patient. In some aspects of the present technology, the system 100 can effectively aspirate clot material from a patient while minimizing blood loss to the patient by reinfusing filtered blood removed therefrom. The system 100 can operate at least partially automatically to reduce operator (e.g., surgeon) operations-thereby reducing errors, improving clot removal efficacy, and decreasing procedure times.

The various operations can be combined and/or performed simultaneously. The computer-readable medium 181 (FIG. 1) of the control system 180 can store a programming sequence/algorithm that, when executed by the processor 182 of the control system 180, can cause the processor 182 to control the system 100 to execute a sequence/algorithm of mechanical operations to perform a method, such as the method described in detail with reference to FIGS. 5A-5H.

FIGS. 6A-6C are a perspective view, a partially-transparent perspective view, and a partially-transparent side view, respectively, of the system 100 in accordance with embodiments of the present technology. Referring to FIGS. 6A-6C, in the illustrated embodiment the collection chamber 110 is positioned on and coupled to (e.g., fixedly coupled to) the housing 141 of the pump assembly 140. The vacuum conduit 123 and the positive pressure conduit 124 (obscured in FIG. 6C) extend between and fluidly couple the collection chamber 110 to the pump assembly 140. The aspiration conduit 121 and the aspiration valve 122 can be coupled to the housing 118 of the collection chamber 110 and/or the housing 141 of the pump assembly 140. The reinfusion conduit 125 can be positioned at least partially within and extend from the housing 141 of the pump assembly 140 to a check valve 691. The one or more user controls 184 (FIG. 1) can comprise a switch 684 configured to power on the system 100 and/or control one or more aspiration and/or reinfusion operations of the system 100.

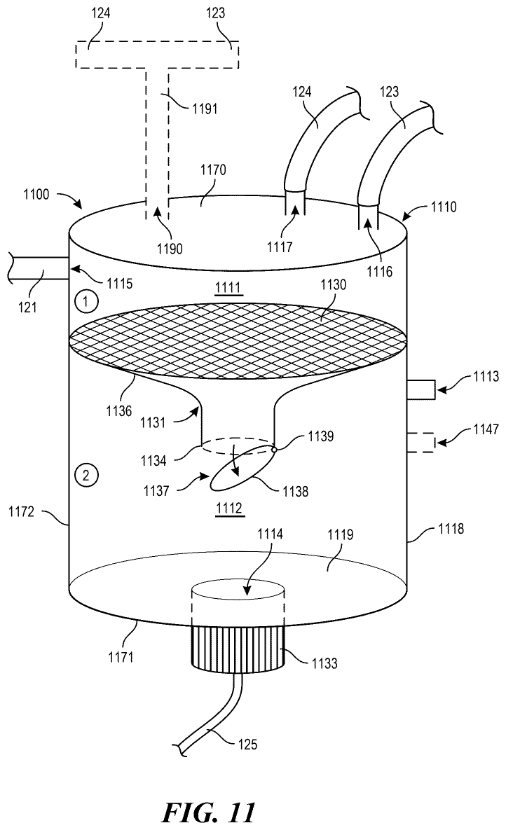

Referring to FIGS. 6B and 6C, the collection component 131 comprises a floor or lower surface of the housing 118 within the chamber 114 such that the lower chamber portion 119b (FIG. 1) is omitted. The collection component 131 directs blood toward the filter 133. Referring to FIGS. 1, 6B, and 6C, some or all of the control system 180, the various valves 122, 126, and 150-153, the pump 142, etc., can be positioned within the housing 141 of the pump assembly 140. For example, as shown in FIGS. 6B and 6C, the power source 183 comprises one or more batteries positioned within the housing 141 and the pump 142 is positioned within the housing 141. Likewise, the vacuum valve 150, the positive pressure valve 151 (obscured in FIGS. 6B and 6C), and the reinfusion valve 126 comprise solenoid valves positioned within the housing 141. The aspiration valve 122 can be an automated large-bore stopcock valve having an actuator assembly 690 positioned within the housing 141, as described in further detail below with reference to FIG. 11. Referring to FIGS. 6A-6C, in some embodiments the system 100 is configured to be entirely disposable. That is, the system 100 can be configured for use in a single clot removal procedure. In other embodiments, the pump assembly 140 can be detached from the collection chamber 110 and reused in subsequent clot removal procedures.

### II. SELECTED EMBODIMENTS OF ADDITIONAL CLOT TREATMENT SYSTEMS, DEVICES, AND METHODS

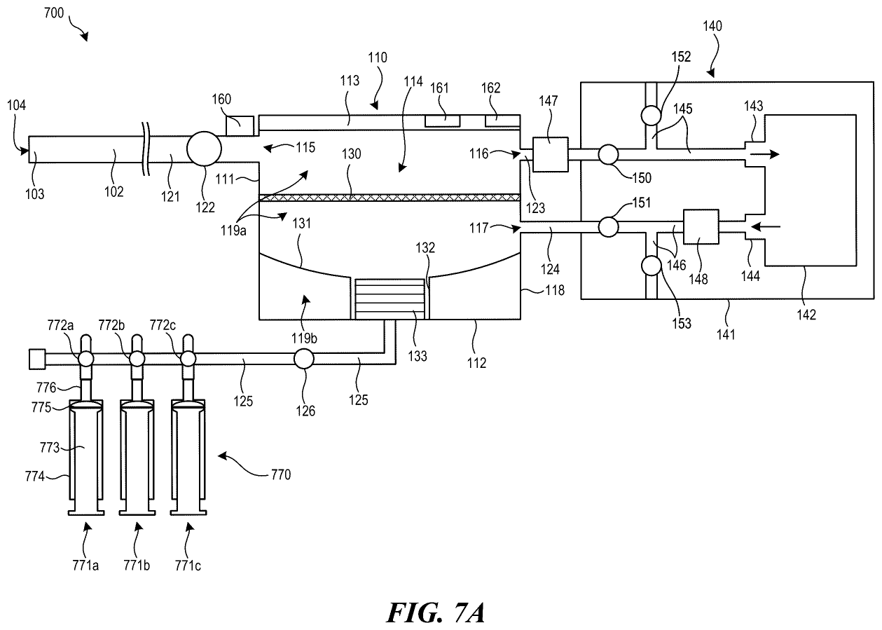

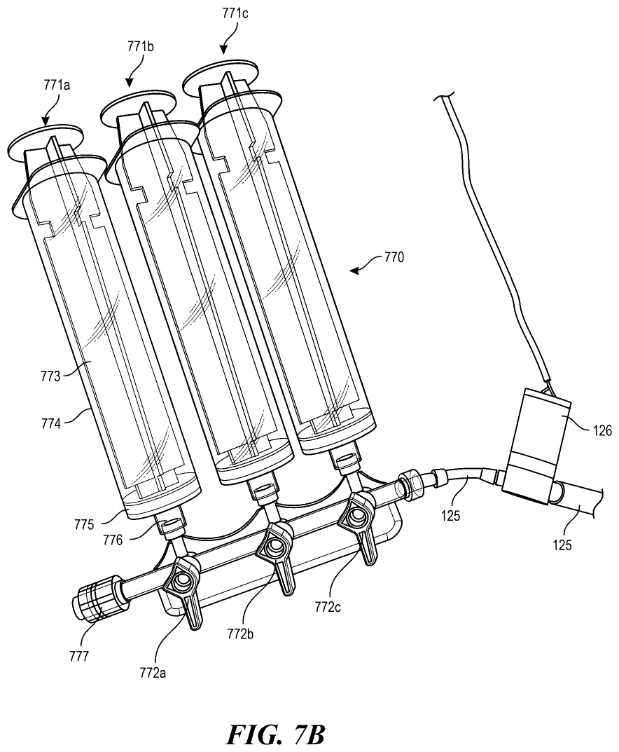

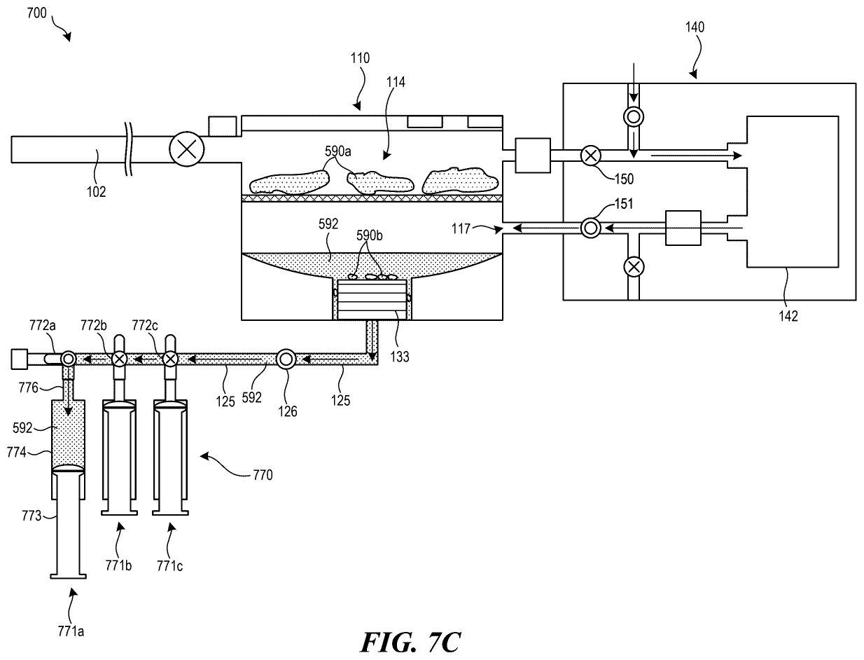

FIG. 7A is a side cross-sectional view of a clot treatment system 700 (“system 700”) in accordance with additional embodiments of the present technology. The system 700 can include several components generally similar or identical to, and can operate in a manner generally similar or identical to, the system 100 described in detail above with reference to FIGS. 1-6C. For example, similar or identical components in FIG. 7A are referred to with the same reference numbers as FIG. 1. Likewise, the system 700 can include the control system 180 (not shown) described in detail above with reference to FIG. 1.

In the illustrated embodiment, the reinfusion conduit 125 is fluidly coupled to a reinfusion manifold 770—rather than the reinfusion catheter 106 (FIG. 1)—via the reinfusion valve 126. FIG. 7B is a perspective of the reinfusion manifold 770 and a portion of the reinfusion conduit 125 including the reinfusion valve 126 in accordance with embodiments of the present technology. Referring to FIG. 7B, the reinfusion valve 126 can comprise a solenoid valve. Referring to FIGS. 7A and 7B, the reinfusion manifold 770 can include one or more syringes 771 (including individually identified first through third syringes 771a-c, respectively) or other containers, pressure sources, etc., each selectively fluidly coupled to the reinfusion conduit 125 via a corresponding syringe valve 772 (including individually identified first through third syringe valves 772a-c, respectively). The reinfusion manifold 770 can comprise one or multiple of the syringes 771, and the syringes 771 can be identical or at least generally identical—each including a plunger 773 slidably positioned within a barrel 774. The plunger 773 can include a seal 775 (e.g., an O-ring) positioned to slidably contact and seal against an interior surface of the barrel 774. The barrels 774 of the syringes 771 can have the same volumes or different volumes, such as a volume equal to or greater than about 30 cc, about 40 cc, about 50 cc, about 60 cc, about 80 cc, about 100 cc, about 150 cc, about 200 cc, and/or the like. The syringes 771 can further include a tip 776 configured to be releasably fluidly coupled to the corresponding one of the syringe valves 772. Accordingly, the syringes 771 can be detached from the reinfusion manifold 770.

The syringe valves 772 can be stopcock or other valves configured to manually operated by a user (e.g., twisted) or automatically operated via the control system 180 (FIG. 1) to move between (i) an open position in which the reinfusion conduit 125 is fluidly coupled to the corresponding one of the barrels 774 of the syringes 771 (e.g., fluid is able to flow from the reinfusion conduit 125 into the corresponding one of the barrels 774) and (ii) a closed position in which the reinfusion conduit 125 is fluidly decoupled from the corresponding one of the barrels 774 of the syringes 771 (e.g., fluid is inhibited or even prevented from flowing from the reinfusion conduit 125 into the corresponding one of the barrels 774). In the illustrated embodiment, each of the syringe valves 772 is in a closed position. In some embodiments, the reinfusion manifold 770 further includes a fluid connector and/or valve 777 coupled to a distal portion of the reinfusion conduit 125 distal of the syringe valves 772. The fluid connector and/or valve 777 can be used to, for example, permit flushing of the system 100.

FIG. 7C is another side cross-sectional view of the clot treatment system 700 of FIGS. 7A and 7B during a reinfusion state of the system 700 in accordance with additional embodiments of the present technology. The system 700 can operate similarly or identically to the system 100 to generate vacuum pressure within the chamber 114 via the pump assembly 140 and apply the vacuum pressure to the aspiration catheter 102 to aspirate the clot material 590 and the blood 592 into the chamber 114 of the collection chamber 110 as described in detail above with reference to FIGS. 5C-5F. Similarly, like the reinfusion state of the system 100 described in detail above with reference to FIG. 5G, the control system 180 (not shown) can open the positive pressure valve 151 and the reinfusion valve 126 such that, as indicated by arrows, the pump 142 then operates to generate positive pressure in the chamber 114 that drives at least a portion of the blood 592 through the filter 133, through the reinfusion conduit 125, through the reinfusion valve 126, and into the reinfusion manifold 770. When one or more of the syringe valves 772 are open, the positive pressure generated by the pump 142 operates to drive the filtered blood 592 into the barrel 774 of the coupled one of the syringes 771. The force of the blood 592 entering the syringes 771 can force the plungers 773 to retract or withdraw through the barrels 774. The syringes 771 can be detached from the reinfusion manifold 770, fluidly coupled to the reinfusion catheter 106 (FIG. 1) via the corresponding one of tips 776, and actuated (e.g., via depression of the plunger 773 through the barrel 774) to drive the filtered blood 592 through the reinfusion catheter 106 into the vasculature of the patient.

More specifically, in the illustrated embodiment the first syringe valve 772a is in the open position such that the filtered blood 592 flows from the reinfusion conduit 125, through the first syringe valve 772a, and into the barrel 774 of the first syringe 771a. In some embodiments, in addition to the positive pressure generated by the pump assembly 140, the plunger 773 can be withdrawn through the barrel 774 to draw the blood 592 from the chamber 114, through the filter 133, through the reinfusion conduit 125, and into the barrel 774 of the first syringe 771a. That is, the blood 592 can be driven through the reinfusion conduit 125 via negative pressure generated by one or more of the syringes 771 and/or via positive pressure generated via the pump assembly 140. When the first syringe 771a is full of the blood 592 and/or contains a desired volume of the blood 592, the first syringe valve 772a can be closed (e.g., manually or automatically). If there is additional blood within the chamber 114 and/or the reinfusion conduit 125, the second syringe valve 772b and/or the third syringe valve 772c can be opened to permit filling of the second syringe 771b and/or the third syringe 771c, respectively. In some embodiments, multiple ones of the syringe valves 772 are open at the same time to permit simultaneous filling of the syringes 771. Any one of the syringes 771 containing the blood 592 can be detached from the reinfusion manifold 770, fluidly coupled to the reinfusion catheter 106 (FIG. 1), and actuated to drive the blood 592 into the reinfusion catheter 106.

Referring to FIGS. 7A-7C, in other embodiments the positive pressure port 117 is omitted from the collection chamber 110 and the outlet 144 of the pump 142 is configured to exhaust fluid (e.g., air) solely to the atmosphere or another source rather than to pressurize the chamber 114. In such embodiments, a user of the system 100 can withdraw the plungers 773 of one or more the syringes 771 to generate negative pressure to draw the blood 592 through the filter 133, into the reinfusion conduit 125, and into the syringes 771 when the corresponding one or more of the syringe valves 772 are open.

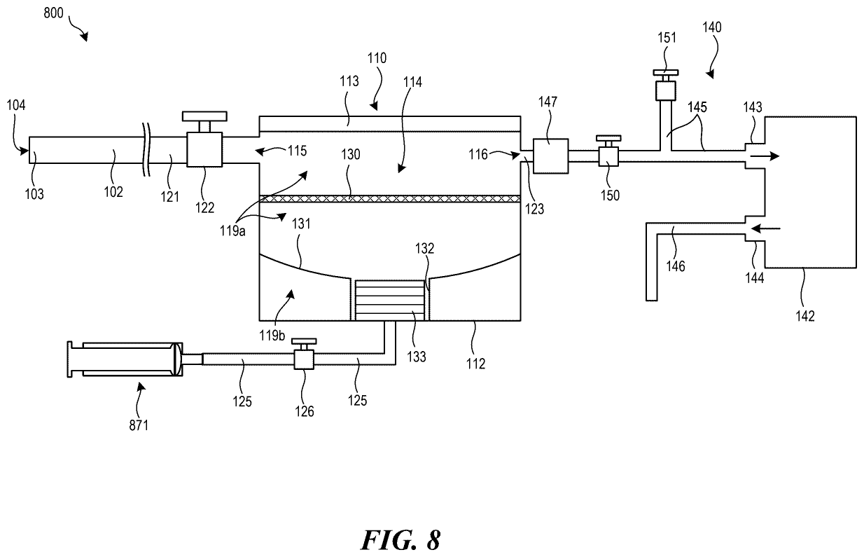

Referring to FIG. 1, one or more components of the system 100 need to be coupled to the control system 180 and, instead, can be operated manually by an operator (e.g., a surgeon, a surgical team member). For example, some or all of the various valves 122, 126, and 150-153 can be configured to be operated manually by a user. FIG. 8, for example, is a side cross-sectional view of a clot treatment system 800 (“system 800”) in accordance with additional embodiments of the present technology. The system 800 can include several components generally similar or identical to, and can operate in a manner generally similar or identical to, the system 100 and/or 700 described in detail above with reference to FIGS. 1-7C. For example, similar or identical components in FIG. 8 are referred to with the same reference numbers as FIG. 1. Likewise, the system 800 can include the control system 180 (not shown) described in detail above with reference to FIG. 1.

In the illustrated embodiment, however, the aspiration valve 122, the reinfusion valve 126, the vacuum valve 150, and the fluid inlet valve 152 are configured as user-actuatable valves, such as stopcock valves, that can be actuated by a user to open/close a fluid path therethrough. Moreover, the positive pressure port 117 is omitted from the collection chamber 110 and the outlet 144 of the pump 142 is configured to exhaust fluid (e.g., air) solely to the atmosphere or another source rather than to pressurize the chamber 114. Accordingly, the reinfusion conduit 125 can be fluidly coupled to a syringe 871, or multiple syringes (e.g., the reinfusion manifold 770 of FIGS. 7A-7C), configured to generate negative pressure to draw filtered blood through the filter 133 for reinfusion in a patient. The system 800 can be operated to aspirate clot material and blood into the chamber 114 and to reinfuse the filtered blood via the syringe 871 in the same or similar manner to that described in detail above with reference to FIGS. 5A-5H-except via user control of the valves 122, 126, 150, and 151 and the syringe 871. In some embodiments, the aspiration valve 122 is a large-bore stopcock valve, such as any of the types described in U.S. patent application Ser. No. 18/182,966, titled “FLUID CONTROL DEVICES FOR CLOT TREATMENT SYSTEMS, AND ASSOCIATED SYSTEMS AND METHODS,” and filed Aug. 22, 2024, which is incorporated by reference herein in its entirety.

FIG. 9 is a side view of an automated stopcock valve 922 in accordance with embodiments of the present technology. The automated stopcock valve 922 can be utilized in the systems 100 and/or 700 described in detail herein (e.g., as an electromechanically controlled valve). For example, the automated stopcock valve 922 can be utilized as the aspiration valve 122 described in detail above.

In the illustrated embodiment, the automated stopcock valve 922 includes a stopcock valve assembly 970 operably coupled to an actuator assembly 990 (e.g., a motor assembly, an electromechanical actuator assembly, a mechanical actuator assembly, and/or the like). The stopcock valve assembly 970 can include a base 971 (shown as transparent in FIG. 9) having a first fluid connector 981 and a second fluid connector 982 and defining a lumen (obscured in FIG. 9) extending therebetween. The base 971 can be coupled between a first tube 911 (e.g., a portion of the aspiration conduit 121 extending between the aspiration valve 122 and the collection chamber 110 of FIG. 1) and a second tube 918 (e.g., a portion of the aspiration conduit 121 extending between the aspiration valve 122 and the aspiration catheter 102 of FIG. 1). The stopcock valve assembly 970 can further include a plunger 973 at least partially positioned within the lumen of the base 971 and at least partially rotatable therein. The plunger 973 can define a lumen or through hole (obscured) and further comprise one or more tabs (e.g., projections, fins) 974 coupled to the plunger 973 and positioned outside the base 971. The through hole of the plunger 973 can have a diameter equal to or greater than about 16 French, about 18 French, about 20 French, about 22 French, about 22 French, about 24 French, and/or the like. The actuator assembly 990 can include a motor 991 operably coupled to an engagement member 992 via a shaft 993. The motor 991 can be a servomotor and/or other type of motor configured to rotate the shaft 993 to rotate the engagement member 992. The engagement member 992 is configured to engage with (e.g., mate with) the plunger 973 such that, when the motor 991 drives the shaft 993 to rotate the engagement member 992, the engagement member 992 correspondingly drives the plunger 973 to rotate within the base 971. For example, the engagement member 992 can define a channel configured to receive the one or more tabs 974 therein. In some embodiments, the automated stopcock valve 922 can further comprise a flush port 976 configured to provide fluid access to the first tube 911 via the first fluid connector 981 and/or to the second tube 918 via the second fluid connector 982.

In the illustrated embodiment, the actuator assembly 990 includes a pair of arms or mounts 995 each having a securement feature 996 at ends thereof. The securement features 996 can each be snapped to or otherwise secured to a corresponding one of the first and second tubes 911, 918 to secure the actuator assembly 990 in position relative to the stopcock valve assembly 970. Accordingly, in some aspects of the present technology the actuator assembly 990 can be secured to a preexisting stopcock valve assembly for motorized control thereof without modification of the stopcock valve assembly. For example, in some embodiments the stopcock valve assembly 970 comprises a stopcock of any of the types described in U.S. patent application Ser. No. 18/182,966, titled “FLUID CONTROL DEVICES FOR CLOT TREATMENT SYSTEMS, AND ASSOCIATED SYSTEMS AND METHODS,” and filed Aug. 22, 2024, which is incorporated by reference herein in its entirety.

In operation, the actuator assembly 990 can be controlled to operate the motor 991 to rotate the plunger 973 within the base 971 (e.g., via the shaft 993 and the engagement member 992) to move the through hole of the plunger 973 into and out of alignment within the first and second fluid connectors 981, 982 to fluidly connect the first tube 911 to the second tube 918 and fluidly disconnect the first tube 911 from the second tube 918, respectively. In some embodiments, the motor 991 is a servomotor that can rotate the shaft 993 by a maximum of about 90 degrees (e.g., incrementally or back and forth about a 90 degrees arc/range). Accordingly, the motor 991 can rotate the through hole of the plunger 973 by about 90 degrees relative to the first and second fluid connectors 981, 982 to bring the through hole into and out of alignment with the first and second fluid connectors 981, 982 to block or provide a fluid path therethrough.

FIG. 10 is a perspective view of an automated stopcock valve 1022 in accordance with additional embodiments of the present technology. The automated stopcock valve 1022 can be utilized in the systems 100 and/or 700 described in detail herein (e.g., as an electromechanically controlled valve). For example, the automated stopcock valve 1022 can be utilized as the aspiration valve 122 described in detail above. Moreover, the automated stopcock valve 1022 can include several features generally similar or identical, and operate generally similarly or identically to, the automated stopcock valve 922 of FIG. 9.

In the illustrated embodiment, the automated stopcock valve 1022 includes a stopcock valve assembly 1070 operably coupled to an actuator assembly 1090 (e.g., a motor assembly, an electromechanical actuator assembly, a mechanical actuator assembly, and/or the like). The stopcock valve assembly 1070 and the actuator assembly 1090 are positioned/secured within a housing 1085 (e.g., between a first housing portion 1086 and a second housing portion 1087 secured together via fasteners, such as screws 1088). The housing 1085 is shown as partially transparent in FIG. 10 for clarity. The housing 1085 can define a lumen 1089 extending therethrough and having an inlet 1082 and an outlet 1081. The stopcock valve assembly 1070 can include a plunger 1073 at least partially positioned within the lumen 1089 and at least partially rotatable therein. The plunger 1073 can define a lumen or through hole 1075. The through hole 1075 and the lumen 1089 can have a diameter equal to or greater than about 16 French, about 18 French, about 20 French, about 22 French, about 22 French, about 24 French, and/or the like. The actuator assembly 1090 can include a motor 1091 operably coupled to a shaft 1093. The shaft 1093 can be directly coupled to (e.g., fixed to) the plunger 1073. The motor 1091 can be a servomotor and/or other type of motor configured to rotate the shaft 1093 to rotate the plunger 1073. In some embodiments, the automated stopcock valve 1022 can further comprise a flush port 1076 configured to provide fluid access through the plunger 1073 to either or both of the inlet 1082 and the outlet 1081 of the lumen 1089.

In operation, the actuator assembly 1090 can be controlled to operate the motor 1091 to rotate the plunger 1073 within the housing 1085 (e.g., via the shaft 1093) to move the through hole 1075 into and out of alignment with the lumen 1089 to fluidly connect the inlet 1082 to the outlet 1081 and fluidly disconnect the inlet 1082 from the outlet 1081, respectively. In some embodiments, the motor 1091 is a servomotor that can rotate the shaft 1093 by a maximum of about 90 degrees (e.g., incrementally or back and forth about a 90 degrees arc/range). Accordingly, the motor 1091 can rotate the through hole 1075 by about 90 degrees relative to the inlet 1082 and the outlet 1081 to bring the through hole into and out of alignment with the inlet 1082 and the outlet 1081 to block or provide a fluid path through the lumen 1089.

In some aspects of the present technology, the automated stopcock valve 1022 is a fully integrated unit. That is, for example, the automated stopcock valve 1022 can replace conventional stopcocks having features for manual operation, such as one or more tabs configured to be grasped and rotated by a user to open and close the stopcock.

### III. SELECTED EMBODIMENTS OF ADDITIONAL CLOT TREATMENT SYSTEMS, DEVICES, AND METHODS INCLUDING FEATURES FOR INHIBITING OR EVEN PREVENTING HEMOLYSIS OF ASPIRATED BLOOD

In some embodiments, a clot treatment system in accordance with the present technology can include one or more features configured to inhibit or even prevent hemolysis of blood aspirated from a patient. Hemolysis is the process of breaking down red blood cells (RBCs) and releasing their contents, primarily hemoglobin, into the surrounding fluid, such as blood plasma. Hemolysis can occur, for example, when blood is exposed to vacuum pressure. Specifically, sustained/prolonged exposure to vacuum pressure can cause blood to boil and hemolyze. At certain levels of hemolysis, blood cannot be reintroduced into a patient without causing negative reactions in the patient. Accordingly, if aspirated blood is subject to excessive vacuum pressure during operation of a clot treatment system it may become unsuitable for reintroduction to a patient. FIGS. 11-27 illustrate several embodiments of clot treatment systems, including features for inhibiting or even preventing hemolysis of blood aspirated from a patient such that the blood remains suitable for reinfusion/reintroduction into the patient.

FIG. 11 is a perspective view of a portion of a clot treatment system 1100 (“system 1100”) including a collection chamber 1110 in accordance with additional embodiments of the present technology. The system 1100 can include several components generally similar or identical to, and can operate in a manner generally similar or identical to any of the systems described in detail above with reference to FIGS. 1-10. For example, similar or identical components in FIG. 11 are referred to with the same reference numbers as FIG. 1. Likewise, the system 1100 can include the control system 180 (not shown) described in detail above with reference to FIG. 1.

In the illustrated embodiment, the collection chamber 1110 includes a housing 1118 that encloses a chamber comprising an upper (e.g., first) chamber 1111 and a lower (e.g., second) chamber 1112. The housing 1118 can be transparent as shown in FIG. 11, or can be opaque/non-transparent. The housing 1118 can be cylindrical as shown in FIG. 11, or can have other shapes (e.g., rectangular prism). In the illustrated embodiment, the cylindrical housing 1118 comprises an upper wall 1170, a lower wall 1171, and a sidewall 1172 extending between the upper wall 1170 and the lower wall 1171. In some embodiments, the lower chamber 1112 can have a larger volume than the upper chamber 1111. In some embodiments, the lower chamber 1112 can have a volume equal to or greater than about 100 cc, about 200 cc, about 300 cc, about 400 cc, about 500 cc, about 600 cc, about 1000 cc, greater than 1000 cc, and/or the like.

The collection chamber 1110 can further include a filter tray 1130 and a collection component 1131 spanning laterally across the housing 1118. In the illustrated embodiment, the collection component 1131 includes (i) a wall portion 1136 (e.g., a collection surface) that slopes downward in a direction toward a central axis of the collection chamber 1110 and (ii) a valve assembly 1137 including a gate valve 1138 hingedly coupled to the wall portion 1136 via a hinge 1139. The collection component 1131 separates the upper chamber 1111 from the lower chamber 1112. The lower wall 1171 can define a collection surface 1119 within the lower chamber 1112 that can be flat as shown in FIG. 11, or that can slope downward toward the central axis of the collection chamber 1110. For example, the collection surface 1119 can have a conical shape.

In some embodiments, the valve assembly 1137 is biased toward a normally-closed/scaled position (not shown in FIG. 11) in which the gate valve 1138 abuts and seals against a lower surface 1134 of the wall portion 1136. In the normally-closed position, the upper chamber 1111 is fluidly disconnected or substantially fluidly disconnected from the lower chamber 1112. The valve assembly 1137 is shown in an open position in FIG. 11. The valve assembly 1137 can be moved to the open position from the normally-closed position by, for example, pressurizing the upper chamber 1111 such that the positive pressure acts against the gate valve 1138 to hinge the gate valve downward (e.g., in a counterclockwise direction shown in FIG. 11) away from the lower surface 1134 of the wall portion 1136 to fluidly connect the upper chamber 1111 to the lower chamber 1112. Although only a single valve assembly 1137 is shown in FIG. 11, in some embodiments the collection component 1131 can comprise multiple valve assemblies of the same or different types.

The housing 1118 can include one or more openings/ports extending through the housing 1118 that provide fluid access to the upper chamber 1111 and/or the lower chamber 1112. For example, in the illustrated embodiment the housing 1118 includes an aspiration port 1115 (e.g., an aspiration inlet) to/from the upper chamber 1111, a vacuum port 1116 (e.g., a vacuum outlet, a negative pressure port, a negative pressure outlet) to/from the upper chamber 1111, a positive pressure port 1117 (e.g., a positive pressure inlet, an exhaust port, an exhaust inlet) to/from the upper chamber 1111, a vent port 1113 to/from the lower chamber 1112, and a reinfusion port 1114 to/from the lower chamber 1112. In the illustrated embodiment, the aspiration port 1115 is fluidly coupled to the aspiration conduit 121, which is fluidly coupled to the aspiration lumen 104 of the aspiration catheter 102 via the aspiration valve 122 as shown in FIG. 1. The vacuum port 1116 can be fluidly coupled to the vacuum conduit 123, which is fluidly coupled to the pump assembly 140 as shown in FIG. 1. Likewise, the positive pressure port 1117 can be fluidly coupled to the positive pressure conduit 124, which is fluidly coupled to the pump assembly 140 as shown in FIG. 1. Similarly, the reinfusion port 1114 can be fluidly coupled to the reinfusion conduit 125, which is fluidly coupled to the reinfusion lumen 108 of the reinfusion catheter 106 as shown in FIG. 1. The vent port 1113 can be provide a vent to atmosphere or ambient pressure.

In some embodiments, the aspiration port 1115 is positioned along the sidewall 1172 of the housing 1118 above the filter tray 1130, and the vacuum port 1116 and the positive pressure port 1117 are positioned along the upper wall 1170 of the housing 1118 above the aspiration port 1115. In the illustrated embodiment, the reinfusion port 1114 is positioned along the lower wall 1171 of the housing 1118 along, for example, the central axis of the collection chamber 1110. The vent port 1113 can be positioned along the sidewall 1172 of the housing 1118 above the reinfusion port 1114, such as vertically near the collection component 1131. In other embodiments, the vent port 1113, the reinfusion port 1114, the aspiration port 1115, the vacuum port 1116, and/or the positive pressure port 1117 (collectively “ports 1113-1117”) can be positioned differently along the housing 1118.

In some embodiments, the vacuum port 1116 and the positive pressure port 1117 can be integrated into a single port. For example, as shown in dashed lines in FIG. 11, in alternative embodiments the housing 1118 can include a single pressure port 1190 to/from the upper chamber 1111 that is fluidly coupled to a pressure conduit 1191. The pressure conduit 1191 can branch via, for example, a T-connector to the positive pressure conduit 124 and the vacuum conduit 123 such that positive pressure and vacuum pressure can be separately generated in the upper chamber 1111 via the pressure conduit 1191 and the pressure port 1190.

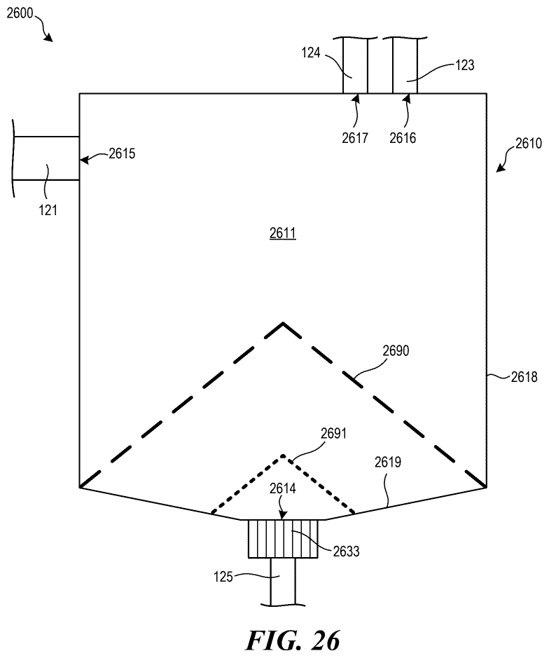

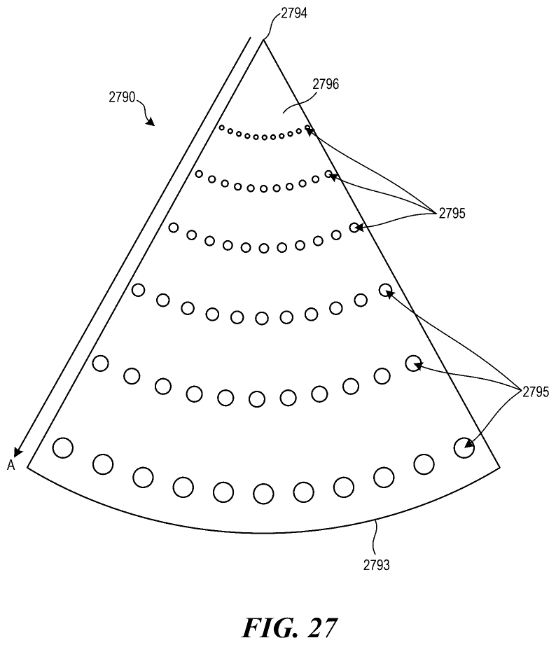

In the illustrated embodiment, the collection chamber 1110 further includes a filter 1133 fluidly coupled between the reinfusion port 1114 and the reinfusion conduit 125. In some embodiments, the filter 1133 defines the reinfusion port 1114. As described in detail above with reference to FIG. 1, the filter tray 1130 can have a first porosity and the filter 1133 can have a second porosity less than the first porosity. Accordingly, the filter tray 1130 can provide a first-stage filtration that filters out larger portions of clot material from blood, while the filter 1133 provides a second-stage filtration that filters out smaller portions of clot material from the blood such that, for example, the blood is suitable for reinfusion to a patient. The filter tray 1130 is planar in FIG. 11. In other embodiments, the filter tray 1130 can have other shapes, such as conical, cylindrical, etc., as described in further detail below with reference to FIGS. 26 and 27.

Operation of the system 1100 is generally described with reference to FIGS. 1 and 11 together. Initially, the control system 180 can move the system 1100 from the first standby state (FIG. 5B) to the vacuum generation state (FIG. 5C) by closing the fluid inlet valve 152 and opening the vacuum valve 150. The pump 142 then operates to draw fluid (e.g., air) from the upper chamber 1111 through the vacuum port 1116 and through the vacuum conduit 123 to generate vacuum in the upper chamber 1111. Referring to FIG. 11, the valve assembly 1137 is in the normally-closed position such that the vacuum pressure is generated only in the upper chamber 1111 and not the lower chamber 1112. Therefore, as the upper chamber 1111 is charged with vacuum, the lower chamber 1112 can remain at ambient pressure. In some embodiments, the vacuum pressure acts against the gate valve 1138 to further draw the gate valve 1138 against the lower surface 1134 of the wall portion 1136 to improve the seal therebetween.

Referring again to FIGS. 1 and 11 together, the control system 180 can move the system 1100 to the aspiration state (FIG. 5E) by opening the aspiration valve 122 to fluidly connect the upper chamber 1111 of the collection chamber 1110 to the aspiration lumen 104 of the aspiration catheter 102 via the aspiration conduit 121. Accordingly, at least a portion of the vacuum stored in the upper chamber 1111 is applied to the aspiration lumen 104 of the aspiration catheter 102 to aspirate clot material and blood through the aspiration lumen 104 of the aspiration catheter 102, through the aspiration valve 122, through the aspiration conduit 121, through the aspiration port 1115, and into the upper chamber 1111. Referring to FIG. 11, the clot material and blood are received through the aspiration port 1115, and gravity causes the clot material and blood to move downward through the upper chamber 1111 toward the filter tray 1130 and the collection component 1131. As described in detail above with reference to FIG. 1, the filter tray 1130 can filter out larger portions of the clot material while allowing the blood and smaller portions of the clot material to pass therethrough. The blood and the smaller portions of the clot material can move downward along the wall portion 1136 toward the gate valve 1138 via gravity.

Referring again to FIGS. 1 and 11 together, the control system 180 can move the system 1100 to a transfer state by opening the positive pressure valve 151 such that the upper chamber 1111 is pressurized by the pump assembly 140 via the positive pressure conduit 124 and the positive pressure port 1117. Referring to FIG. 11, the positive pressure acts to move the valve assembly 1137 from the normally-closed position to the open position such that the blood and smaller portions of the clot material can flow therethrough from the upper chamber 1111 to the lower chamber 1112 and collect on the collection surface 1119. That is, in some aspects of the present technology positive pressure in the upper chamber 1111 is used to transfer fluid (e.g., blood and clot material) from the upper chamber 1111 to the lower chamber 1112 rather than, for example, a weight of the fluid and/or gravity.

Referring again to FIGS. 1 and 11 together, the control system 180 can move the system 1100 to the first standby state by closing the positive pressure valve 151. Referring to FIG. 11, pressure is then equalized in the lower chamber 1112 via the vent port 1113 until the pressure in the upper chamber 1111 and the lower chamber 1112 are equal, thereby allowing the valve assembly 1137 to return to the normally-closed position in which the upper chamber 1111 and the lower chamber 1112 are fluidly disconnected. At this point, the blood and the smaller portions of the clot material are stored in the lower chamber 1112 at ambient pressure.

Referring again to FIGS. 1 and 11 together, the control system 180 can then control the system 1100 to again cycle one or more times through the vacuum generation state, the aspiration state, the transfer state, and the first standby state, to (i) aspirate additional blood and clot material into the upper chamber 1111 and (ii) move/transfer the blood and clot material from the upper chamber 1111 to the lower chamber 1112. After each cycle (e.g., aspiration pass), more blood is stored in the lower chamber 1112. Referring to FIG. 11, in some aspects of the present technology the valve assembly 1137 inhibits or even prevents blood stored in the lower chamber 1112 from being exposed to vacuum during the vacuum generation state and the aspiration state, which could cause hemolysis of the blood. Specifically, the valve assembly 1137 fluidly disconnected the upper chamber 1111 from the lower chamber 1112 when vacuum pressure is generated in the collection chamber 1110 (e.g., the upper chamber 1111 thereof) such that the vacuum is not applied to the blood stored in the lower chamber 1112. Instead, blood stored in the lower chamber 1112 from a previous aspiration operation is stored at ambient pressure which is advantageously not expected to cause hemolysis or substantial hemolysis of the blood.

Referring again to FIGS. 1 and 11 together, when an operator of the system 1100 (e.g., a physician) is ready to reinfuse blood collected in the lower chamber 1112 to the patient, the control system 180 can move the system 1100 to the reinfusion state by opening the positive pressure valve 151 and the reinfusion valve 126. The pump 142 then operates to pressurize the upper chamber 1111 via the positive pressure conduit 124 and the positive pressure port 1117, causing the valve assembly 1137 to open and fluidly connect the upper chamber 1111 to the lower chamber 1112. The positive pressure generated by the pump 142 is then applied to the lower chamber 1112 to drive/force the blood and the smaller portions of the clot material toward the filter 1133 and the reinfusion conduit 125. As described in detail above with reference to FIG. 1, the filter 1133 filters out the smaller portions of the clot material from the blood such that the blood is suitable for reinfusion into the patient. The positive pressure drives the blood through the filter 1133, through the reinfusion conduit 125, through the reinfusion valve 126, and at least partially through the reinfusion lumen 108 of the reinfusion catheter 106. At least some of the filtered blood can exit the reinfusion lumen 108 into the vasculature of the patient. In other embodiments, the reinfusion conduit 125 can be connected to a syringe or other container configured to receive and retain the filtered blood.

Referring to FIG. 11, in some embodiments the positive pressure port 1117 can be a first positive pressure port 1117 to the upper chamber 1111 and the housing 1118 of the system 1100 can further include a second positive pressure port 1147 (shown in dashed lines) to/from the lower chamber 1112. The second positive pressure port 1147 can be fluidly coupled to a source of positive pressure configured to generate positive pressure in the lower chamber 1112 independently of any pressure (positive or negative) generated in the upper chamber 1111. For example, the second positive pressure port 1147 can be coupled to the pump assembly 140 (FIG. 1) via the same positive pressure conduit 124 as the first positive pressure port 1117 via a valved connection (e.g., via an electromechanical valve) such that positive pressure can be independently or simultaneously routed (i) through the positive pressure conduit 124 to the upper chamber 1111 via the first positive pressure port 1117 and/or (ii) to the lower chamber 1112 via the second positive pressure port 1147. In some embodiments, the second positive pressure port 1147 is coupled to a separate conduit (other than the positive pressure conduit 124) that is fluidly coupled to the pump assembly 140 (FIG. 1). In other embodiments, the second positive pressure port 1147 is coupled to a source of positive pressure different than the pump assembly 140, such as a separate pressure source (e.g., a syringe, electrical pump, etc.). In such embodiments, the second positive pressure conduit 1147 can facilitate simultaneous aspiration and reinfusion within the system 1100. For example, the system 1100 can perform the vacuum generation state and/or the vacuum aspiration states described above while the second positive pressure conduit 1147 simultaneously receives positive pressure that pressurizes the lower chamber 1112 to drive blood through the filter 1133, through the reinfusion conduit 125, through the reinfusion valve 126, and at least partially through the reinfusion lumen 108 of the reinfusion catheter 106. In some aspects of the present technology, simultaneous aspiration and reinfusion can reduce the duration of a thrombectomy procedure using the system 1100. And, such an arrangement including two positive pressure sources that can operate independently to pressurize a lower chamber and an upper chamber can be utilized in other embodiments disclosed herein having upper and lower chambers that can be fluidly disconnected.

Referring to FIG. 11, although the valve assembly 1137 is shown as a gate valve, the valve assembly 1137 can include other types of valves that inhibit or even prevent fluid flow from the upper chamber 1111 to the lower chamber 1112 when vacuum is generated in the upper chamber 1111. For example, the valve assembly 1137 can comprise a one-way check valve, such as a duckbill valve, an umbrella valve, a cross-slit valve, a dome valve, a ball valve, electronic valve, and/or another type of valve that (i) permits fluid flow from the upper chamber 1111 to the lower chamber 1112 but that (ii) inhibits or even prevents fluid flow from the lower chamber 1112 to the upper chamber 1111.

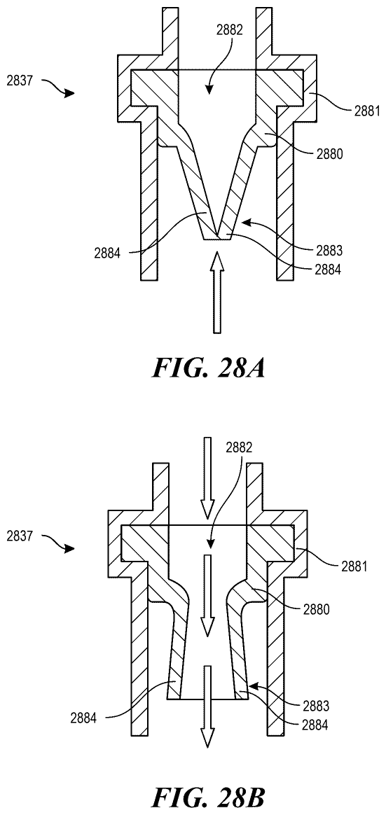

FIGS. 28A and 28B, for example, are side cross-sectional views of a duckbill valve assembly 2837 (“duckbill valve 2837”) in a closed position and an open position, respectively, that can be utilized in the system 1100 of FIG. 11 and/or other systems described herein in accordance with embodiments of the present technology. Referring to FIGS. 28A and 28B, in the illustrated embodiment the duckbill valve 2837 includes a flexible (e.g., elastomeric) tube 2880 secured to/within a housing 2881 (e.g., a valve seat). The tube 2880 defines a lumen 2882 and can have a flattened end portion 2883 defining opposing lips 2884 (e.g., edges) shaped in a way that resembles the bill of a duck. The lips 2884 abut and seal against one another in the closed position (FIG. 28A) to inhibit or even prevent flow through the lumen 2882, and the lips 2884 part in response to a positive pressure in the lumen 2882 to permit flow through the lumen 2882 (FIG. 28B). Referring to FIGS. 11, 28A, and 28B, the duckbill valve 2837 can be positioned within the system 1100 such that the flattened end portion 2883 faces the second chamber 1112 such that the duckbill valve 2837 is (i) normally-closed (FIG. 28A) to inhibit or even prevent fluid flow from the lower chamber 1112 to the upper chamber 1111 and can (ii) open (FIG. 28B) in response to positive pressure in the upper chamber 1111 to allow fluid (e.g., blood and clot material) to pass through the lumen 2882 from the upper chamber 1111 to the lower chamber 1112.

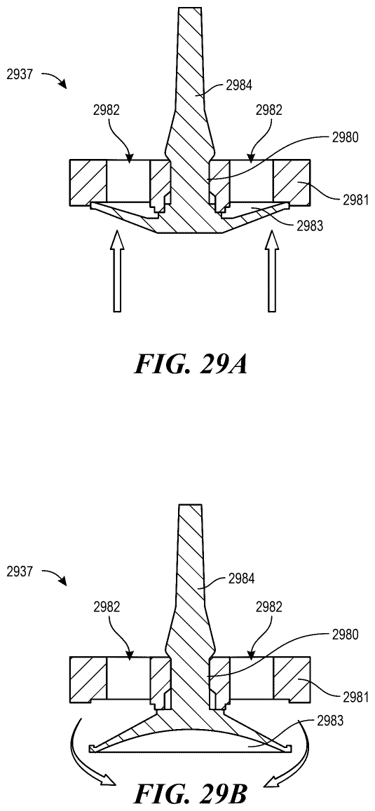

FIGS. 29A and 29B are side cross-sectional views of an umbrella valve assembly 2937 (“umbrella valve 2937”) in a closed position and an open position, respectively, that can be utilized in the system 1100 of FIG. 11 and/or other systems described herein in accordance with embodiments of the present technology. Referring to FIGS. 29A and 29B, in the illustrated embodiment the umbrella valve 2937 includes a flexible (e.g., elastomeric) diaphragm 2980 secured to a housing 2981 (e.g., a valve seat; e.g., sealingly secured within a channel thereof). The housing 2981 can include one or more openings 2982 (e.g., an annular groove) extending therethrough. The diaphragm 2980 can be shaped like an umbrella having, for example, a scaling portion 2983 (e.g., a circular portion) connected to a stem 2984 that extends through the opening 2982. The scaling portion 2983 can abut seal against the housing 2981 in the closed position (FIG. 29A) to inhibit or even prevent flow through the opening 2982, and the sealing portion 2983 can flex away from the housing 2981 in response to a positive pressure in the lumen 2882 to permit flow through the opening 2982. The diaphragm 2980 can resemble an inside-out umbrella in the open position. Referring to FIGS. 11, 29A, and 29B, the umbrella valve 2937 can be positioned within the system 1100 such that the scaling portion 2983 faces the second chamber 1112 such that the umbrella valve 2937 is (i) normally-closed (FIG. 29A) to inhibit or even prevent fluid flow from the lower chamber 1112 to the upper chamber 1111 and can (ii) open (FIG. 29B) in response to positive pressure in the upper chamber 1111 to allow fluid (e.g., blood and clot material) to pass through the opening 2982 from the upper chamber 1111 to the lower chamber 1112.

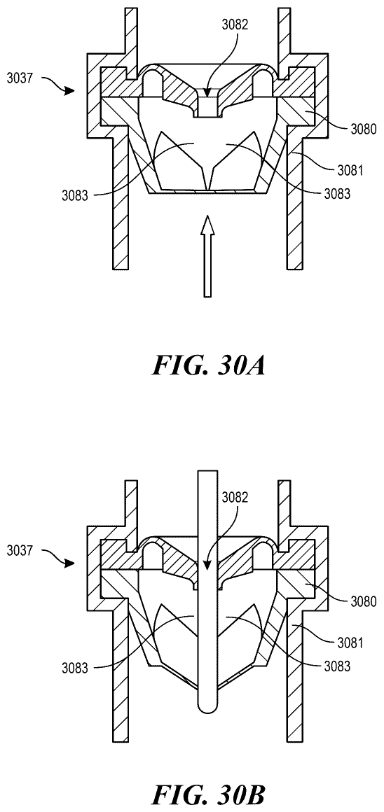

FIGS. 30A and 30B are side cross-sectional views of a cross-slit valve assembly 3037 (“cross-slit valve 3037”) in a closed position and an open position, respectively, that can be utilized in the system 1100 of FIG. 11 and/or other systems described herein in accordance with embodiments of the present technology. Referring to FIGS. 30A and 30B, in the illustrated embodiment the cross-slit valve 3037 includes a flexible (e.g., elastomeric) diaphragm 3080 secured to/within a housing 3081 (e.g., a valve seat). The diaphragm 3080 can define a lumen 3082 and have a pair of slits dividing the diaphragm into four cusps 3083 (only two of which are visible in the cross-sectional views of FIGS. 30A and 30B). The cusps 3083 abut and seal against one another in the closed position (FIG. 30A) to inhibit or even permit flow through the lumen 3082, and the cusps 3083 part in response to a positive pressure in the lumen 3082 to allow flow through the lumen 3082 (FIG. 30B). The cusps 3083 could also part if an instrument were passed therethrough as shown in FIG. 30B. Referring to FIGS. 11, 30A, and 30B, the cross-slit valve 3037 can be positioned within the system 1100 such that the cusps 3083 face the second chamber 1112 such that the cross-slit valve 3037 is (i) normally-closed (FIG. 30A) to inhibit or even prevent fluid flow from the lower chamber 1112 to the upper chamber 1111 and can (ii) open (FIG. 30B) in response to positive pressure in the upper chamber 1111 to allow fluid (e.g., blood and clot material) to pass through the lumen 3082 from the upper chamber 1111 to the lower chamber 1112. In some aspects of the present technology, the cross-slit valve 3037 allows for a greater volume/flow therethrough as compared to, for example, the duckbill valve 2837 of FIGS. 28A and 28B.

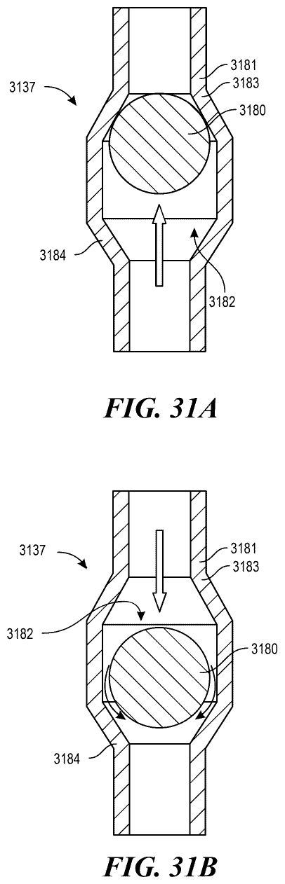

FIGS. 31A and 31B are side cross-sectional views of a ball valve assembly 3137 (“ball valve 3137”) in a closed position and an open position, respectively, that can be utilized in the system 1100 of FIG. 11 and/or other systems described herein in accordance with embodiments of the present technology. Referring to FIGS. 31A and 31B, in the illustrated embodiment the ball valve 3137 includes a ball 3180 secured to/within a housing 3181 (e.g., a valve seat). More specifically, the ball 3180 can be constrained to move (e.g., float freely) within a chamber 3182 (e.g., an orificc) having a first end portion 3183 and a second end portion 3184. The ball 3180 and the chamber 3182 can be sized/shaped such that the ball 3180 abuts and seals against the first end portion 3183 of the chamber 3182 in the closed position (FIG. 31A) to inhibit or even permit flow through the chamber 3182, and such that the ball 3180 moves away from the first end portion 3183 toward the second end portion 3184 but does not seal against the second end portion 3184 in response to a positive pressure to allow flow through the chamber 3182 (FIG. 31B). Referring to FIGS. 11, 31A, and 31B, the valve 3137 can be positioned within the system 1100 such that the second end portion 3184 faces the second chamber 1112 such that the ball valve 3137 is (i) closed (FIG. 31A) to inhibit or even prevent fluid flow from the lower chamber 1112 to the upper chamber 1111 when a negative pressure exists in the upper chamber 1111 and can (ii) open (FIG. 31B) in response to positive pressure in the upper chamber 1111 to allow fluid (e.g., blood and clot material) to pass through the chamber 3182 from the upper chamber 1111 to the lower chamber 1112.

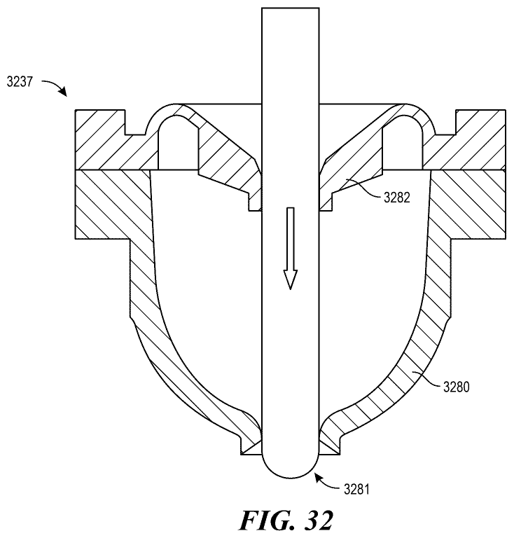

FIG. 32 is a side cross-sectional view of a dome valve assembly 3237 (“dome valve 3237”) in an open position that can be utilized in the system 1100 of FIG. 11 and/or other systems described herein in accordance with embodiments of the present technology. In the illustrated embodiment, the dome valve 3237 includes a flexible (e.g., elastomeric) dome 3280 having one or more slits/openings 3281 formed therein. Similar to a cross-slit valve or duckbill valve, the slit(s) 3281 can seal in the absence of a positive pressure, and can open in response to a positive pressure to permit fluid to pass therethrough. The dome valve 3237 can further comprise a cross-slit valve 3282 or other type of valve to provide a back-up seal and redundancy. The dome valve 3237 and cross-slit valve 3282 can open in response to positive pressure and/or if an instrument were passed therethrough as shown in FIG. 32.

Referring to FIG. 11, in yet other embodiments the valve assembly 1137 can comprise an electronic valve such as a solenoid valve, a stopcock valve, a gate valve, a ball valve, a butterfly valve, a pinch valve, and/or another type of valve that can be controlled by the control system 180 (FIG. 1) to open and close to provide the functionality described with reference to FIG. 11.

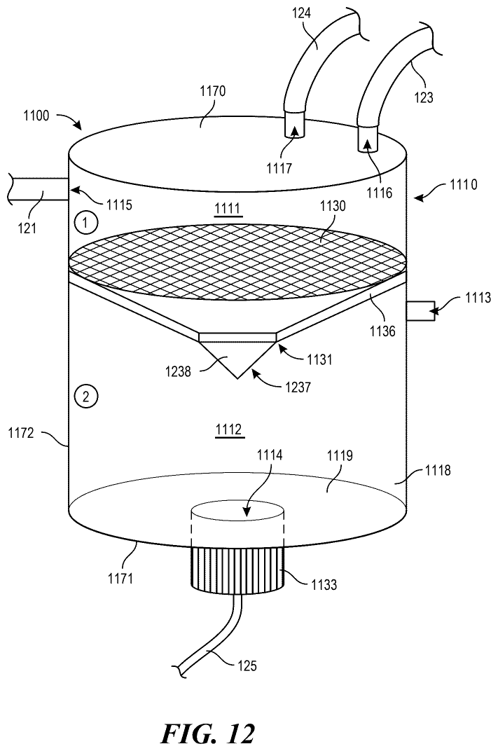

FIG. 12, for example, is another perspective view of the portion of the clot treatment system 1100 including a different valve assembly 1237 that comprises a duckbill check-valve (e.g., check valve) in accordance with additional embodiments of the present technology. The duckbill check valve 1237 can have some features generally similar or identical in structure and/or function to the duckbill valve 2837 of FIGS. 28A and 28B. For example, the valve assembly 1237 can comprise a flexible (e.g., elastomeric) tube 1238 with a flattened end that is (i) normally-closed to inhibit or even prevent fluid flow from the lower chamber 1112 to the upper chamber 1111 and that (ii) opens in response to positive pressure in the upper chamber 1111 to allow fluid to pass from the upper chamber 1111 to the lower chamber 1112. The wall portion 1136 of the collection component 1131 can slope downward toward the central axis of the system 1100 and the duckbill check-valve 1137 to facilitate movement of blood toward the duckbill check-valve 1137. Although only a single duckbill check-valve 1137 is shown in FIG. 12, in other embodiments the collection chamber 1110 can include multiple check valves each configured to provide a one-way fluid path from the upper chamber 1111 to the lower chamber 1112.

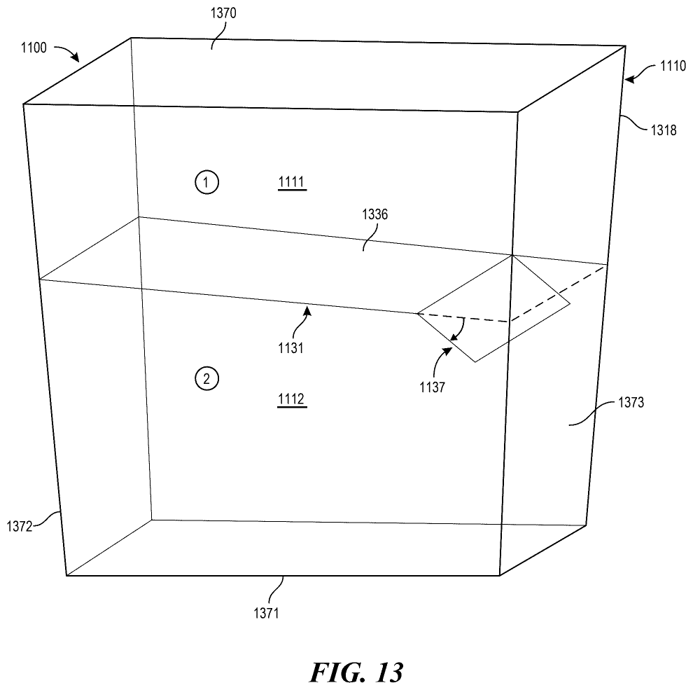

Referring to FIGS. 11 and 12, the wall portion 1136 of the collection component 1131 can be generally conical and the valve assembly 1137/1237 can be positioned along the central axis of the collection chamber 1110. In other embodiments, the collection component 1131 can have other geometries. For example, FIG. 13 is a perspective view of the portion of the clot treatment system 1100 in which the collection chamber 1110 includes a housing 1318 and a collection component 1331 having different geometries in accordance with embodiments of the present technology. In the illustrated embodiment, the housing 1318 has a generally rectangular prism shape including, in part, an upper wall 1370, a lower wall 1371 opposite the upper wall 1370, a first sidewall 1372, and a second sidewall 1373 opposite the first sidewall 1372. In the illustrated embodiment, the collection component 1331 includes a wall portion 1336 that separates the upper chamber 1111 from the lower chamber 1112 and that slopes downward linearly in a direction from the first sidewall 1372 toward the second sidewall 1373. The valve assembly 1137 (e.g., gate-valve) is positioned adjacent the second sidewall 1373 below a lowermost portion of the wall portion 1336.

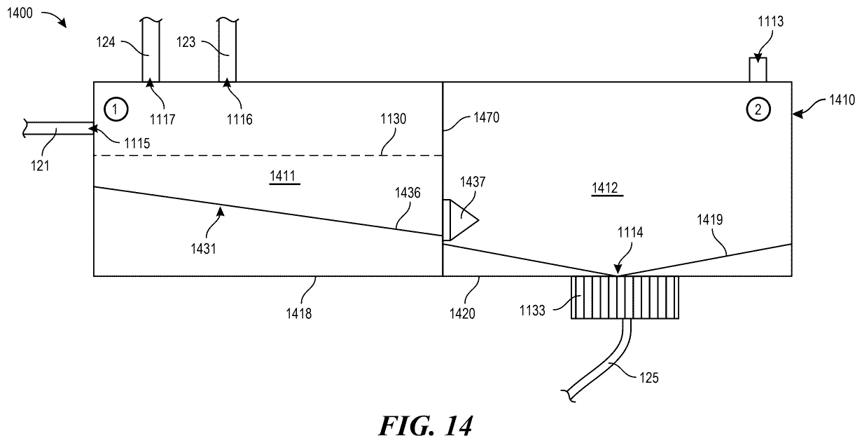

FIG. 14 is a side view of a portion of a clot treatment system 1400 (“system 1400”) in accordance with additional embodiments of the present technology. The system 1400 can include several components generally similar or identical to, and can operate in a manner generally similar or identical to, the system 1100 described in detail above with reference to FIGS. 11-13. For example, similar or identical components in FIG. 14 are referred to with the same reference numbers as FIGS. 11-13. Likewise, the system 1400 can include the control system 180 (not shown) described in detail above with reference to FIGS. 1 and 11-13.

In the illustrated embodiment, the system includes a collection chamber 1410 having a first chamber 1411 and a second chamber 1412 positioned side-by-side rather than one-over-the-other. In some embodiments, a dividing wall 1470 can separate the first chamber 1411 from the second chamber 1412. The filter tray 1130 can be positioned in the first chamber 1411 and the first chamber 1411 can include/comprise a first housing 1418 that includes the aspiration port 1115 to the aspiration conduit 121, the vacuum port 1116 to the vacuum conduit 123, and the positive pressure port 1117 to the positive pressure conduit 124. In the illustrated embodiment, a collection component 1431 is positioned in the first chamber 1411 and comprises a wall portion 1436 and a valve assembly 1437. The wall portion 1436 can slope downward in a direction toward the valve assembly 1437 and the second chamber 1412. The valve assembly 1437 can be a one-way check valve (e.g., a duckbill valve) that is (i) normally-closed to inhibit or even prevent fluid flow from the second chamber 1412 to the first chamber 1411 and that (ii) opens in response to positive pressure in the first chamber 1411 to allow fluid to pass from the first chamber 1411 to the second chamber 1412. The valve assembly 1437 can be positioned in/along the dividing wall 1470 to provide a one-way fluid path therethrough from the first chamber 1411 to the second chamber 1412. In other embodiments, the valve assembly 1437 can be other types of valves, such as a gate valve described above with reference to FIGS. 1 and 13.

The second chamber 1412 can include/comprise a second housing 1420 that includes the vent port 1113 to atmosphere/ambient pressure and the reinfusion port 1114 to the filter 1133 and the reinfusion conduit 1125. In the illustrated embodiment, a collection surface 1419 is positioned in the second chamber 1412 and can have, for example, a conical shape that slopes radially inward toward the reinfusion port 1114.

Referring to FIGS. 1, 11, and 14 together, the control system 180 can move the system 1400 between the vacuum generation, aspiration, transfer, and standby states to aspirate blood and clot material into the first chamber 1411 and move (e.g., transfer) blood and smaller portions of clot material to the second chamber 1412 for storage. In some aspects of the present technology, the valve assembly 1437 inhibits or even prevents blood stored in the second chamber 1412 from being exposed to vacuum during the vacuum generation state and the aspiration state, which could cause hemolysis of the blood. The control system 180 can then move the system 1400 to the reinfusion state to drive blood from the second chamber 1412 through the filter 1133 and the reinfusion conduit 125 for reintroduction to a patient. In some aspects of the present technology, the second chamber 1412 can be positioned side-by-side with the first chamber 1411 because positive pressure is used to drive aspirated blood from the first chamber 1411 to the second chamber 1412 rather than gravity or a weight of the aspirated material.

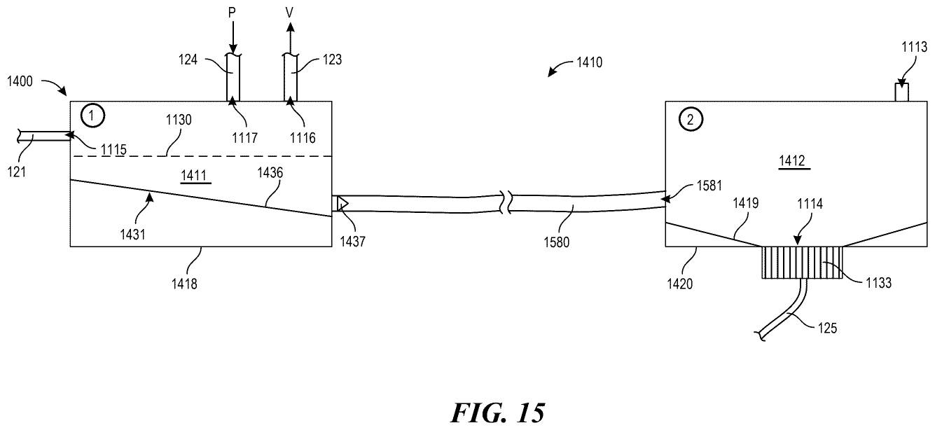

In other embodiments, the first chamber 1411 can be fluidly connected to the second chamber 1412 via the valve assembly 1437 through tubing and/or in different manners. FIG. 15, for example, is another side view of the portion of the clot treatment system 1400 in which the first chamber 1411 is spaced apart from the second chamber 1412 and connected thereto via tubing 1580 in accordance with additional embodiments of the present technology. In the illustrated embodiment, the tubing 1580 provides a fluid path from the valve assembly 1437 of the first chamber 1411 to a blood and clot inlet port 1581 of the second housing 1420 of the second chamber 1412. In some embodiments, the tubing 1580 is flexible such that the first chamber 1411 and the second chamber 1412 can be positioned differently relative to one another (e.g., with the second chamber 1412 above, below, or at the same vertical level relative to the first chamber 1411). In some embodiments, the tubing 1580 permits the first chamber 1411 and the second chamber 1412 to be spaced apart from each other by a distance of about 1 foot, about 2 feet, about 3 feet, about 4 feet, about 5 feet, or greater than 5 feet.

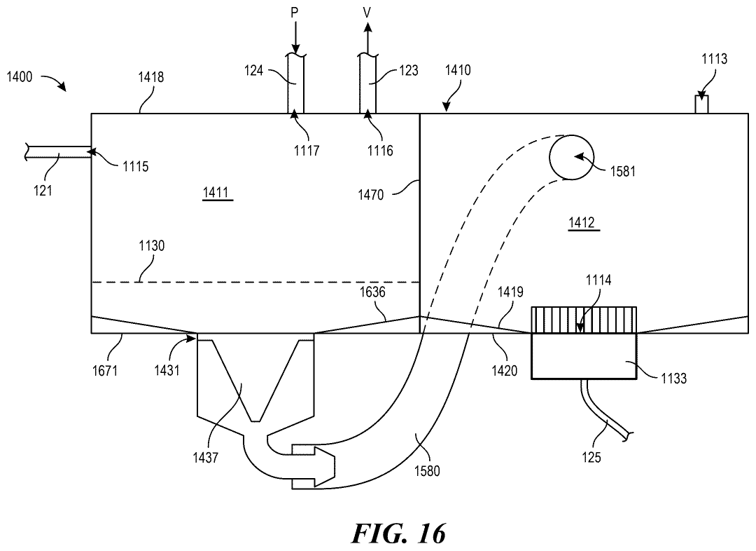

FIG. 16 is another side view of the portion of the clot treatment system 1400 in which the first chamber 1411 is positioned side-by-side with the second chamber 1412 and connected thereto via the tubing 1580 in accordance with additional embodiments of the present technology. In the illustrated embodiment, the first chamber 1411 is positioned side-by-side with the second chamber 1412 and separated therefrom by the dividing wall 1490, and the valve assembly 1437 extends along/through a lower wall 1671 of the first housing 1418 of the first chamber 1411. The collection component 1431 can further include a wall portion 1636 that is conical (e.g., funneled) and/or that slopes downward toward the valve assembly 1437. The tubing 1580 provides a fluid path from the valve assembly 1437 of the first chamber 1411 to the blood and clot inlet port 1581 of the second housing 1420 of the second chamber 1412.

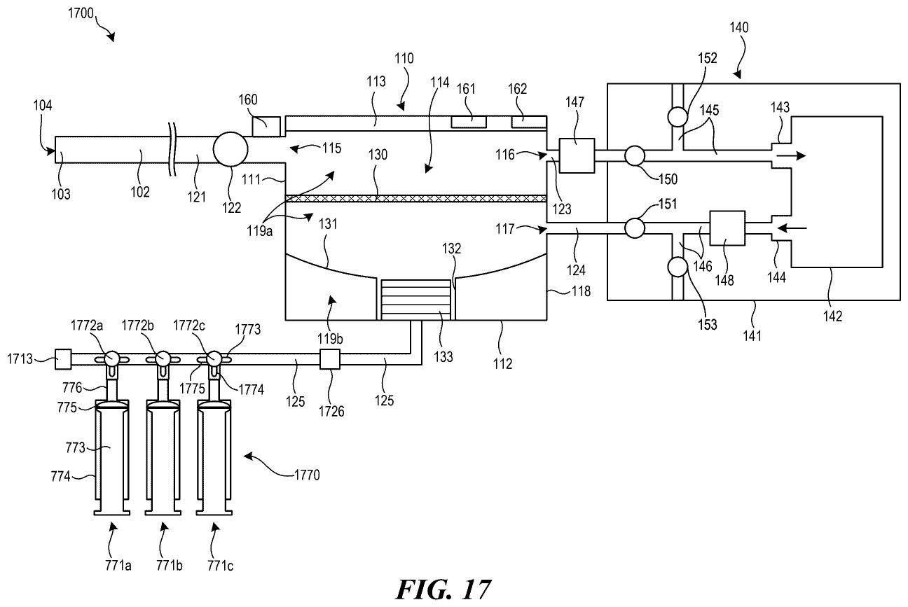

FIG. 17 is a side cross-sectional view of a clot treatment system 1700 (“system 1700”) in accordance with additional embodiments of the present technology. The system 1700 can include several components generally similar or identical to, and can operate in a manner generally similar or identical to, the system 100 and/or the system 700 described in detail above with reference to FIGS. 1-7C. For example, similar or identical components in FIG. 7A are referred to with the same reference numbers as FIGS. 1 and 7A-7C. Likewise, the system 700 can include the control system 180 (not shown) described in detail above with reference to FIG. 1.

In the illustrated embodiment, the reinfusion conduit 125 is fluidly coupled to a reinfusion manifold 1770—rather than the reinfusion catheter 106 (FIG. 1)—via a one-way reinfusion valve 1726. The one-way reinfusion valve 1726 can be duckbill valve, check valve, umbrella valve, and/or the like. The reinfusion manifold 1770 can include the one or more syringes 771, each fluidly coupled to the reinfusion conduit 125 via a corresponding syringe valve 1772 (including individually identified first through third syringe valves 1772a-c, respectively). The syringe valves 7172 can be three-way check valves each having an inlet 1773, a low-pressure outlet 1774 to the corresponding one of the syringes 771, and a high-pressure outlet 1775 that allows fluid to flow distally past the corresponding one of the syringes 771. In some embodiments, the reinfusion manifold 1770 further includes a vent 1713 coupled to a distal portion of the reinfusion conduit 125 distal of the syringe valves 1772.

The system 1700 can operate similarly or identically to the system 100 to generate vacuum pressure within the chamber 114 via the pump assembly 140 and apply the vacuum pressure to the aspiration catheter 102 to aspirate clot material and blood into the chamber 114 of the collection chamber 110 as described in detail above with reference to FIGS. 5C-5F. After each aspiration, like the reinfusion state of the system 100 described in detail above with reference to FIG. 5G, the control system 180 (not shown) can open the positive pressure valve 151 such that the pump 142 then operates to generate positive pressure in the chamber 114 that drives at least a portion of the aspirated blood through the filter 133, through the reinfusion conduit 125, through the one-way reinfusion valve 1726, and into the reinfusion manifold 1770. Initially, the blood is driven into the third syringe valve 1772c, which routes the blood through the low-pressure outlet 1774 until the third syringe 771c is full, at which point additional blood is driven through the high-pressure outlet 1775 toward the second syringe valve 1772b, which operates similarly. Accordingly, as additional blood is driven into the reinfusion manifold 1770, the syringes 771 can sequentially fill. At any point, the syringes 771 can be detached from the reinfusion manifold 1770, fluidly coupled to the reinfusion catheter 106 (FIG. 1) via the corresponding one of tips 776, and actuated (e.g., via depression of the plunger 773 through the barrel 774) to drive the filtered blood through the reinfusion catheter 106 into the vasculature of the patient. In some aspects of the present technology, by driving aspirated blood from the chamber 114 into the syringes 771 after each aspiration pass, the blood is not stored within the chamber 114 when additional vacuum is generated therein for a subsequent aspiration operation. Instead, the blood is stored in the syringes 771 at pressures (e.g., ambient pressure) that inhibit or even prevent hemolysis of the blood.

In some embodiments, the diameters of the reinfusion conduit 125 and or the syringe valves 1772 can vary and have, for example, stepped diameters. Additionally, the reinfusion manifold 1770 can include one or more flush lines (not shown).

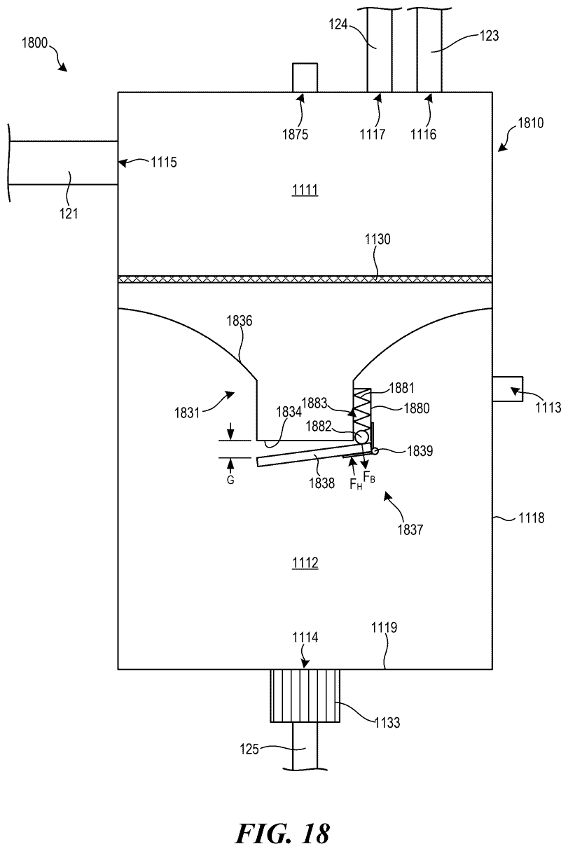

In some embodiments, a collection chamber in accordance with the present technology can include a collection component configured to pass blood from a first (e.g., vacuum) chamber to a second (e.g., storage) chamber via the force of gravity in addition to or alternatively to a force from a positive pressure generated in the first chamber. FIG. 18, for example, is a side cross-sectional view of a portion of a clot treatment system 1800 (“system 1800”) including a collection chamber 1810 in accordance with additional embodiments of the present technology. The system 1100 can include several components generally similar or identical to, and can operate in a manner generally similar or identical to, at least, the system 1100 described in detail above with reference to FIG. 11. For example, similar or identical components in FIG. 18 are referred to with the same reference numbers as FIG. 11. Likewise, the system 1100 can include the control system 180 (not shown) described in detail above with reference to FIG. 1.

In the illustrated embodiment, the collection chamber 1810 includes a collection component 1831 spanning laterally across the housing 1118 and separating the upper chamber 1111 from the lower chamber 1112. In addition to the ports 1113-1117, the housing 1118 can further include a vent port 1875 to/from the upper chamber 1111. In some embodiments, the vent port 1875 can be selectively opened and closed via the control system 180 (FIG. 1) and/or can be fluidly coupled the pump assembly 140 (FIG. 1) to provide a connection to ambient pressure. In some embodiments, the vent port 1875 can be integrated into and/or its functionality provided by the positive pressure port 1117. In other embodiments, the vent port 1875 can be omitted entirely.

The collection component 1831 can include a wall portion 1836 that slopes downward in a direction toward a central axis of the collection chamber 1110 and a valve assembly 1837. The valve assembly can include a gate valve 1838, a hinge 1839, a biasing member mount 1880 fixed to the wall portion 1836, and a plunger 1883 comprising a biasing member 1881 mounted to the biasing member mount 1880 and an engagement member 1882 fixed to the biasing member 1881. The hinge 1839 is fixed between the biasing member mount 1880 and the gate valve 1838 such that the gate valve can hingedly move relative to the biasing member mount 1880 and the wall portion 1836. The biasing member 1881 can be a compression spring or similar member configured to exert a biasing force in a downward direction (e.g., in a direction from the upper chamber 1111 toward the lower chamber 1112). The engagement member 1882 can be a ball or similar member configured to engage and transmit a force from the biasing member 1881 to the gate valve 1838. Accordingly, the plunger 1883 can be a ball-nose plunger.

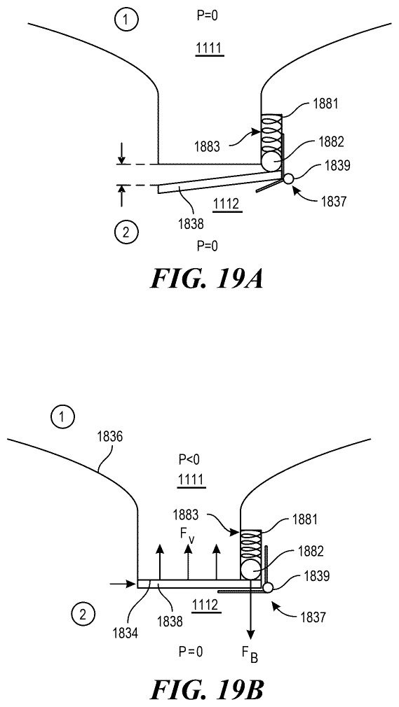

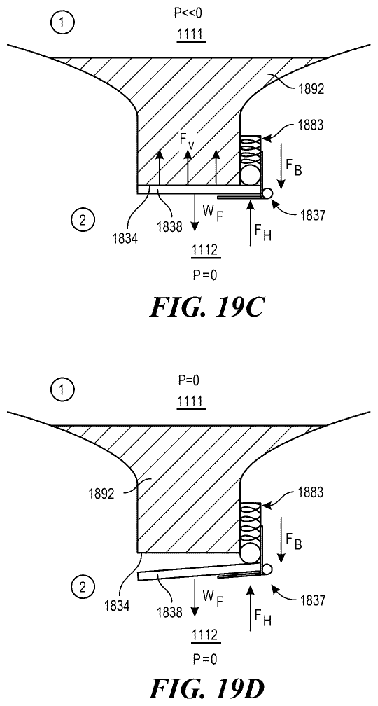

In the illustrated embodiment, the valve assembly 1837 is shown in a resting state when a pressure in the upper chamber 1111 is equal to or about equal to a pressure in the lower chamber 1112. For example, the valve assembly 1837 can assume the resting state when both the upper chamber 1111 and the lower chamber 1112 are at ambient pressure. In the resting state, the valve assembly 1837 is in an open position in which the gate valve 1838 is spaced apart from a lower surface 1834 of the wall portion 1836 by a small gap G such that the upper chamber 1111 is fluidly connected to the lower chamber 1112. As described in further detail below with reference to FIGS. 19A-19D, the valve assembly 1837 is movable to a closed/sealed position in which the gate valve 1838 abuts and seals against the lower surface 1834 of the wall portion 1836.

More specifically, the plunger 1833 can exert a plunger force F_{B} against the gate valve 1838 that acts to rotate the gate valve 1838 in a first (e.g., counterclockwise) direction away from the lower surface 1834, while the hinge 1839 can exert a hinge force F_{H} that acts to rotate the gate valve 1838 in an opposite second (e.g., clockwise) direction toward the lower surface 1834. In the resting state, the plunger force F_{B} is greater than the hinge force F_{H} such that the gate valve 1838 is in the open position spaced apart from the lower surface 1834 by the gap G.

FIGS. 19A-19D are side views of the collection component 1831 of the system 2100 of FIG. 18 in the resting state, a charged vacuum state, a filled with vacuum state, and a vented state, respectively, during operation system 2100 in accordance with embodiments of the present technology. The resting state shown in FIG. 19A is the same as that shown in FIG. 18.

Referring to FIGS. 1, 18, and 19B together, the control system 180 can move the system 1800 from the resting state (FIGS. 18 and 19A) to the charged vacuum state of FIG. 19B (e.g., the vacuum generation state shown in FIG. 5C) by closing the fluid inlet valve 152 and opening the vacuum valve 150 such that the pump 142 draws fluid from the upper chamber 1111 through the vacuum port 1116 and through the vacuum conduit 123 to generate vacuum in the upper chamber 1111. Referring to FIG. 19B, the vacuum pressure generated in the upper chamber 1111 exerts a vacuum force F_{V} against the gate valve 1138 that acts to rotate the gate valve 1838 in the second direction toward the lower surface 1834 to the closed position in which the gate valve 1838 abuts and seals against the lower surface 1834 of the wall portion 1836 to fluidly disconnect the upper chamber 1111 from the lower chamber 1112. That is, the combined vacuum force Fᵥ and hinge force F_{H} can be greater than the plunger force F_{B} (Fᵥ+F_{H}>F_{B}).

Referring to FIGS. 1, 18, and 19C together, the control system 180 can move the system 1800 to the filled with vacuum state (e.g., the aspiration state shown in FIG. 5E) by opening the aspiration valve 122 to fluidly connect the upper chamber 1111 of the collection chamber 1810 to the aspiration lumen 104 of the aspiration catheter 102 via the aspiration conduit 121 such that at least a portion of the vacuum stored in the upper chamber 1111 is applied to the aspiration lumen 104 of the aspiration catheter 102 to aspirate clot material and blood 1892 through the aspiration lumen 104 of the aspiration catheter 102, through the aspiration valve 122, through the aspiration conduit 121, through the aspiration port 1115, and into the upper chamber 1111. Referring to FIG. 19C, the blood 1892 (and clot material) exerts a fluid force W_{F} via the weight of the fluid that acts to rotate the gate valve 1838 in the first direction away from the lower surface 1834. In the filled with vacuum state, the gate valve 1838 can remain in the closed position as the combined vacuum force F_{V} and hinge force F_{H} is greater than the combined plunger force F_{B} and fluid force W_{F} (F_{V}+F_{H}>F_{B}+W_{F}).

Referring to FIGS. 1, 18, and 19D together, the control system 180 can move the system 1800 to the vented state by venting the upper chamber 1111 to atmosphere via the vent port 1875 or by otherwise equalizing the pressure between the upper chamber 1111 and the lower chamber 1112. Referring to FIG. 19D, in the vented state the combined plunger force F_{B} and fluid force W_{F} is greater than the hinge force F_{H} such that the valve assembly 1837 moves to the open position in which the gate valve 1838 is spaced apart from the lower surface 1834. This permits the blood 1892 to drain via gravity from the upper chamber 1111 into the lower chamber 1112. Because the gate valve 1838 remains open in the resting state, the valve assembly 1837 can permit all of the blood 1892 to drain into the lower chamber 1112. That is, in some aspects of the present technology opening of the valve assembly 1837 is not reliant on the weight of the fluid on the gate valve 1838 (the fluid force W_{F}) such that all of the blood 1892 can pass through the valve assembly 1837 into the lower chamber 1112.

Referring to FIGS. 1 and 18-19D together, the control system 180 can control the system 1800 to conduct one more additional aspiration cycles (e.g., including the resting state, the charged vacuum state, the filled with vacuum state, and the vented state) to aspirate additional blood and clot material and move the blood and smaller portions of the clot material from the upper chamber 1111 to the lower chamber 1112. After each cycle (e.g., aspiration pass), more blood is stored in the lower chamber 1112. Referring to FIGS. 18-19D, in some aspects of the present technology the valve assembly 1837 inhibits or even prevents blood stored in the lower chamber 1112 from being exposed to vacuum when vacuum is generated in the upper chamber 1111 by automatically closing in response to the vacuum force F_{V} exerted against the gate valve 1838, which inhibits or even prevents hemolysis of the blood. Specifically, the valve assembly 1837 fluidly disconnected the upper chamber 1111 from the lower chamber 1112 when vacuum pressure is generated in the collection chamber 1810 (e.g., the upper chamber 1111 thereof) such that the vacuum is not applied to the stored blood. Instead, blood stored in the lower chamber 1112 from a previous aspiration operation is stored at ambient pressure which advantageously is not expected to cause hemolysis or substantial hemolysis of the blood.

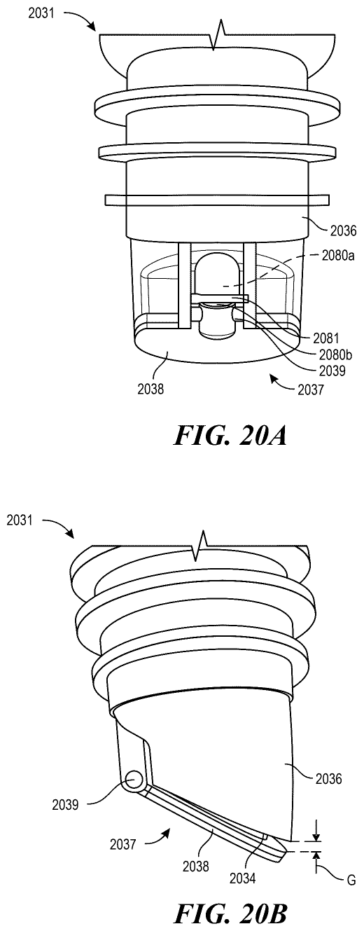

FIGS. 20A and 20B are a rear view and a side view, respectively, of a collection component 2031 that can be used in the clot treatment system 1800 of FIG. 18 in accordance with additional embodiments of the present technology. The collection component 2031 can include several components generally similar or identical to, and can operate in a manner generally similar or identical to, at least, the collection component 1831 described in detail above with reference to FIGS. 18-19D. For example, in the illustrated embodiment the collection component 2031 includes a wall portion 2036 (e.g., a funnel portion) and a valve assembly 2037. The valve assembly 2037 can include a gate valve 2038 hingedly coupled to the wall portion 2036 via a hinge 2039. Referring to FIG. 20A, the valve assembly 2037 can further include a first magnet 2080a (obscured in FIG. 20A) mounted to the wall portion 2036, a second magnet 2080b mounted to the gate valve 2038 in opposing relation to the first magnet 2080a, and a biasing member 2081 positioned between the first and second magnets 2080a-b. The biasing member 2081 can comprise, for example, a foam spring.

Referring to FIGS. 20A and 20B together, the valve assembly 2037 is shown in a resting state, such as when a pressure in the upper chamber 1111 (FIG. 18) is equal to a pressure in the lower chamber 1112 (FIG. 18). In the resting state, the valve assembly 2037 is in an open position in which the gate valve 2038 is spaced apart from a lower surface 2034 (FIG. 20B) of the wall portion 2036 by a small gap G (FIG. 20B) such that the upper chamber 1111 is fluidly connected to the lower chamber 1112. In the resting position, the biasing member 2081 exerts a force against the gate valve 2038 to open the valve assembly 2037 by pivoting the gate valve 2038 away from the lower surface 2034 while the first and second magnets 2080a-b exert an opposite force against the valve assembly 2037 by pivoting the gate valve 2038 toward the lower surface 2034. The magnetic force of the first and second magnets 2080a-b and the biasing force of the biasing member 2081 can be selected such that, in the resting position, the gap G is relatively small (e.g., the gate valve 2038 is slightly ajar). In some embodiments, the hinge 2039 exerts little or no force against the gate valve 2038. Like the valve assembly 1837 described in detail above with reference to FIGS. 18-29D, the resting state enables blood to completely drain through the valve assembly 2037 without relying on the weight thereof to open the valve assembly 2037. Likewise, the valve assembly 2037 is movable to a closed/scaled position in which the gate valve 2038 abuts and seals against the lower surface 2034 of the wall portion 2036 when a vacuum is generated in the upper chamber 1111 above the gate valve 2038. This can inhibit or even prevent vacuum from being applied to blood stored below the gate valve 2038 in the lower chamber 1112.

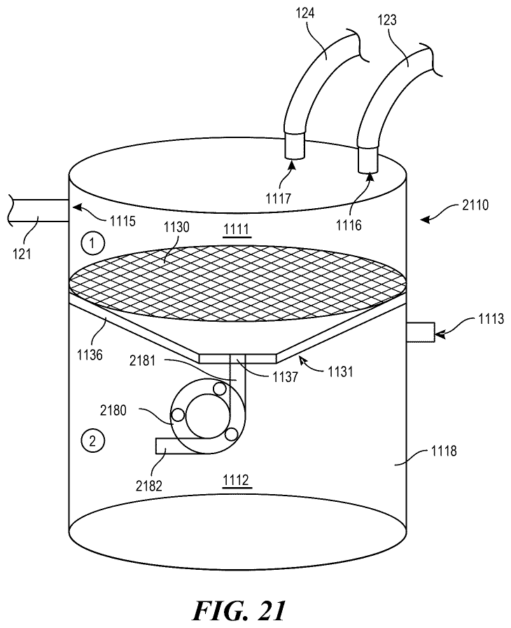

FIG. 21 is a perspective view of a portion of a clot treatment system 2100 (“system 2100”) in accordance with additional embodiments of the present technology. The system 2100 can include several components generally similar or identical to, and/or can operate in a manner generally similar or identical to, the system 1100 described in detail above with reference to FIGS. 11-13. For example, similar or identical components in FIG. 21 are referred to with the same reference numbers as FIGS. 11-13. Likewise, the system 2100 can include the control system 180 (not shown) described in detail above with reference to FIGS. 1 and 11-13.

In the illustrated embodiment, the system 2100 includes a collection chamber 2110 having the collection component 1131, including the valve assembly 1137, which is a one-way check valve. In the illustrated embodiment, the collection chamber 2110 further includes a pump 2180 positioned in the lower chamber 1112 and having an inlet 2181 fluidly coupled to the valve assembly 1137 and an outlet 2182 into the lower chamber 1112. The pump 2180 can be a peristaltic pump, roller pump, centrifugal pump, and/or other pump configured to generate vacuum pressure at the inlet 2181 and positive pressure at the outlet 2182 to move fluid into the inlet 2181 and to and through the outlet 2182.

In operation, the upper chamber 1111 can be charged with vacuum via the vacuum port 1116 and the vacuum conduit 123 while the valve assembly 1137 inhibits or even prevents application of that vacuum to the lower chamber 1112, as described in detail above. The vacuum can then be applied to the aspiration catheter 102 (FIG. 1) via the aspiration port 1115 and the aspiration conduit 121 to aspirate blood and clot material into the upper chamber 1111. In some embodiments, the control system 180 (FIG. 1) can then activate the pump 2180 to move the blood (and smaller portions of the clot material) through the valve assembly 1137, through the pump 2180, and into the lower chamber 1112. Accordingly, in some aspects of the present technology the pump 2180 operates to move blood from the upper chamber 1111 to the lower chamber 1112 via the force generated thereby rather than via pressurization of the upper chamber 1111 or via the force of gravity. Blood can be stored in the lower chamber 1112 during subsequent aspiration operations at ambient pressure without exposure to vacuum via the one-way fluid path between the upper chamber 1111 and the lower chamber 1112 provided by the valve assembly 1137. In some embodiments, the housing 1118 can include a reinfusion port (not shown) and filter (not shown) coupled to a reinfusion conduit (not shown) to allow the stored blood to be reinfused into a patient, as described in detail above.

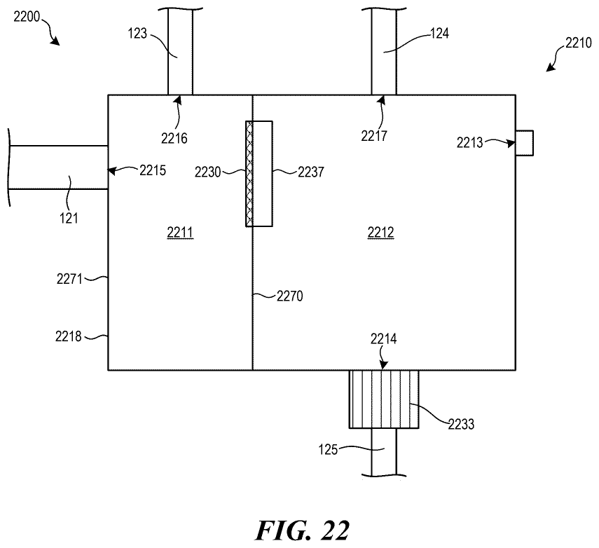

In yet other embodiments, clot treatment systems in accordance with the present technology can transfer aspirated material (e.g., blood) between chambers using aspirational flow rates rather than pressure differentials. For example, FIG. 22 is a side cross-sectional view of a portion of a clot treatment system 2200 (“system 2200”) including a collection chamber 2210 in accordance with additional embodiments of the present technology. The system 2200 can include several components generally similar or identical to, and can operate in a manner generally similar or identical to, any of the systems described in detail above with reference to FIGS. 1-21. For example, similar or identical components in FIG. 22 are referred to with the same reference numbers as FIG. 1. Likewise, the system 2200 can include the control system 180 (not shown) described in detail above with reference to FIG. 1.

In the illustrated embodiment, the collection chamber 2210 includes a housing 2218 that encloses a first chamber 2211 and a second chamber 2222. In the illustrated embodiment, the first chamber 2211 is positioned side-by-side with the second chamber 2212 and separated therefrom by a dividing wall 2270. In some embodiments, the second chamber 2212 can have a volume equal to or greater than about 100 cc, about 200 cc, about 300 cc, about 400 cc, about 500 cc, about 600 cc, about 1000 cc, greater than 1000 cc, and/or the like.

The collection chamber 2210 can further include a filter plate 2230 and a valve assembly 2237 coupled to the dividing wall 2270. The valve assembly 2237 can comprise a one-way check valve, such as a flexible duckbill valve or umbrella valve that (i) permits fluid flow from the first chamber 2211 to the second chamber 2212 but that (ii) inhibits or even prevents fluid flow from the second chamber 2212 to the first chamber 2211. The filter plate 2230 can be provided on a side of the filter plate 2230 facing the first chamber 2211.

The housing 2218 can include one or more openings/ports extending through the housing 2218 that provide fluid access to the first chamber 2211 and/or the second chamber 2212. For example, in the illustrated embodiment the housing 2218 includes an aspiration port to/from the first chamber 2211, a vacuum port 2216 to/from the first chamber 2211, a positive pressure port 2217 to/from the second chamber 2212, a vent port 2213 to/from the second chamber 2222, and a reinfusion port 2214 to/from the second chamber 2212. In the illustrated embodiment, the aspiration port 2215 is fluidly coupled to the aspiration conduit 121, which is fluidly coupled to the aspiration lumen 104 of the aspiration catheter 102 via the aspiration valve 122 as shown in FIG. 1. The vacuum port 2216 can be fluidly coupled to the vacuum conduit 123, which is fluidly coupled to the pump assembly 140 as shown in FIG. 1. Likewise, the positive pressure port 2217 can be fluidly coupled to the positive pressure conduit 124, which is fluidly coupled to the pump assembly 140 as shown in FIG. 1. Similarly, the reinfusion port 2214 can be fluidly coupled to the reinfusion conduit 125, which is fluidly coupled to the reinfusion lumen 108 of the reinfusion catheter 106 as shown in FIG. 1. The vent port 2213 can be provide a vent to atmosphere.

In some embodiments, the aspiration port 2215 is provided in a sidewall 2271 of the housing 2218 at a vertical level that is above or about equal to a vertical position of the filter plate 2230 and the valve assembly 2237. In the illustrated embodiment, the collection chamber 2210 further includes a filter 2233 fluidly coupled between the reinfusion port 2214 and the reinfusion conduit 125. In some embodiments, the filter 2233 defines the reinfusion port 2214. Similar to the filter tray 130 described in detail above with reference to FIG. 1, the filter plate 2230 can have a first porosity and the filter 2233 can have a second porosity less than the first porosity. Accordingly, in operation, the filter plate 2230 can provide a first-stage filtering of larger portions of clot material while the filter 2233 provides a second-stage filtering of smaller portions of clot material.

Operation of the system 2200 is generally described with reference to FIGS. 1 and 22. Initially, the control system 180 can control the system 2200 to generate and store vacuum pressure in the first chamber 2211 by closing the aspiration valve 122, closing the fluid inlet valve 152 and opening the vacuum valve 150. The pump 142 then operates to draw fluid (e.g., air) from the first chamber 2211 through the vacuum port 2216 and through the vacuum conduit 123 to generate vacuum in the first chamber 2211. The valve assembly 2237 inhibits or even prevents vacuum generated in the first chamber 2211 from being applied to the second chamber 2222.

Next, the control system 180 can open the aspiration valve 122 to fluidly connect the first chamber 2211 of the collection chamber 2210 to the aspiration lumen 104 of the aspiration catheter 102 via the aspiration conduit 121 such that at least a portion of the vacuum stored in the first chamber 2211 is applied to the aspiration lumen 104 of the aspiration catheter 102 to aspirate clot material and blood through the aspiration lumen 104 of the aspiration catheter 102, through the aspiration valve 122, through the aspiration conduit 121, through the aspiration port 2215, and into the first chamber 2211. In some aspects of the present technology, a flow rate of the aspirated material can be relatively large, such as equal to or greater than about 30 cubic centimeters per second (cc/sec), greater than about 40 cc/sec, greater than about 50 cc/sec, greater than about 60 cc/sec, greater than about 70 cc/sec, greater than about 80 cc/sec, greater than about 90 cc/sec, greater than about 100 cc/sec, greater than about 120 cc/sec, greater than about 150 cc/sec, greater than about 200 cc/sec, greater than about 250 cc/sec, greater than about 300 cc/sec, greater than about 400 cc/sec, greater than about 500 cc/sec, or greater. The generation of such high flow rates using stored vacuum pressure are described in, for example, U.S. Pat. No. 11,559,382, titled “SYSTEM FOR TREATING EMBOLISM AND ASSOCIATED DEVICES AND METHODS,” and filed Aug. 8, 2019, which is incorporated herein by reference in its entirety.

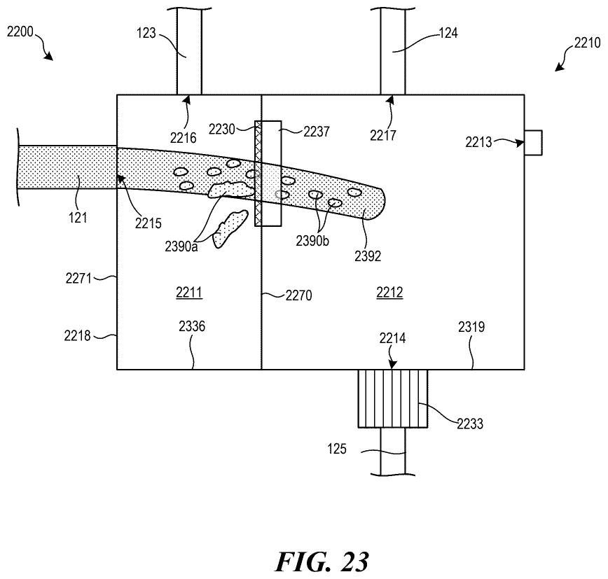

The high flow rate aspirated material can traverse laterally across the first chamber 2211 and impact the filter plate 2230 and the valve assembly 2237. The filter plate 2230 is configured (e.g., has as porosity, size, and/or shape) to filter out larger portions of the clot material while the impact of the aspirated material forcibly opens the valve assembly 2237, allowing blood and smaller portions of the clot material to pass through the valve assembly 2237 into the second chamber 2212. FIG. 23, for example, is another side cross-sectional view of the portion of the clot treatment system 2200 illustrating the flow of clot material 2390 (including individually identified larger portions 2390a and smaller portions 2390b) and blood 2392 through the collection chamber 2210 during aspiration in accordance with embodiments of the present technology. In the illustrated embodiment, the blood 2392 and clot material 2390 aspirated through the aspiration conduit 121 enter the first chamber 2211 through the aspiration port 2215 at a high flow rate (e.g., as a fast-moving jet or stream) and impinge upon the filter plate 2230 and the valve assembly 2237. The filter plate 2230 can filter out the larger portions 2390a of the clot material, which can remain in the first chamber 2211, before the larger portions 2390a contact the valve assembly 2237. For example, the larger portions 2390a of the clot material can move downward away from the filter plate 2230 via gravity and collect within the first chamber 2211, such as on a collection surface 2336 of the first chamber 2211.

The blood 2392 and smaller portions 2390b of the clot material physically engage the valve assembly 2237 to move the valve assembly 2237 to an open position that allows fluid flow from the first chamber 2211 to the second chamber 2222. For example, these components exert a force on the valve assembly 2237 proportional to their flow rate to force the valve assembly 2237 to the open position. Vacuum in the first chamber 2211 can exert a force against the valve assembly 2237 that tends to close the valve assembly 2237. However, in some embodiments the level of vacuum in the first chamber 2211 rapidly decays as the blood 2392 and clot material 2390 are aspirated into the first chamber 2211 such that the force of the blood 2392 and the smaller portions 2390b of the clot material against the valve assembly 2237 overpowers the vacuum force to open the valve assembly 2237 and permit the blood 2392 and the smaller portions 2390b of the clot material to pass into the second chamber 2212. The blood 2392 and the smaller portions 2390b of the clot material can then collect in the second chamber 2212, such as on a collection surface 2319 of the second chamber 2212.

Referring to FIGS. 1, 22, and 23 together, the control system 180 can control the system 2200 to conduct one more additional aspiration cycles of charging vacuum within the first chamber 2211 and subsequently applying the charged vacuum to the aspiration catheter 102 to aspirate additional blood and clot material into the first chamber 2211 and against the valve assembly 2237 at high flow rate, permitting the blood to pass through the valve assembly 2237 and into the second chamber 2212 and to collect in the second chamber 2212. After each cycle (e.g., aspiration pass), more blood is stored in the second chamber 2212. In some aspects of the present technology, the valve assembly 2237 inhibits or even prevents blood stored in the second chamber 2212 from being exposed to vacuum when vacuum is generated in the first chamber 2211 (e.g., by normally-closing and opening only when the blood contacts the valve assembly 2237 at high flow rate), which inhibits or even prevents hemolysis of the blood. Specifically, the valve assembly 2237 fluidly disconnects the first chamber 2211 from the second chamber 2212 when vacuum pressure is generated in the collection chamber 2210 (e.g., the first chamber 2211 thereof) such that the vacuum is not applied to the blood stored in the second chamber 2212. Instead, blood stored in the second chamber 2212 from a previous aspiration operation is stored at ambient pressure, which advantageously is not expected to cause hemolysis or substantial hemolysis of the blood.

When an operator of the system 2200 (e.g., a physician) is ready to reinfuse blood collected in the second chamber 2212 to the patient, the control system 180 can move the system 2200 to a reinfusion state by opening the positive pressure valve 151 and the reinfusion valve 126. The pump 142 then operates to pressurize the second chamber 2212 via the positive pressure conduit 124 and the positive pressure port 2217 to drive/force the blood and the smaller portions of the clot material toward the filter 2233 and the reinfusion conduit 125. As described in detail above with reference to FIG. 1, the filter 2233 filters out the smaller portions of the clot material from the blood such that the blood is suitable for reinfusion into the patient. The positive pressure drives the blood through the filter 2233, through the reinfusion conduit 125, through the reinfusion valve 126, and at least partially through the reinfusion lumen 108 of the reinfusion catheter 106. At least some of the filtered blood can exit the reinfusion lumen 108 into the vasculature of the patient.

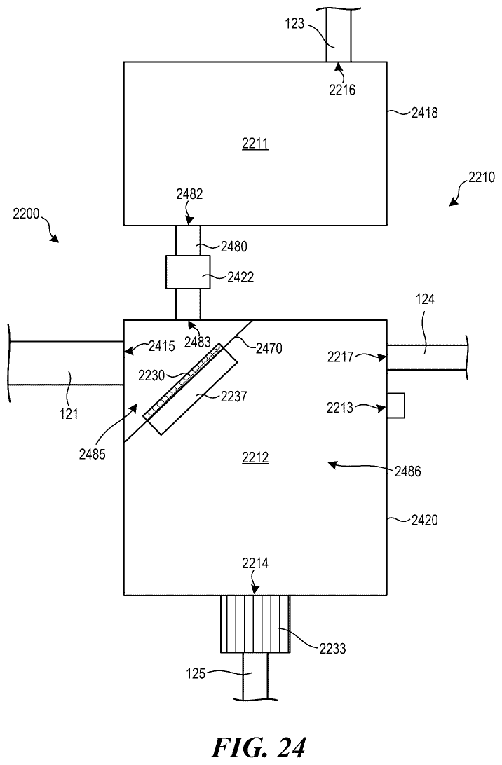

In other embodiments, the first chamber 2211 can be fluidly connected to the second chamber 2212 via the valve assembly 2237 through tubing and/or in different manners. FIG. 24, for example, is another side view of the portion of the clot treatment system 2200 in which the first chamber 2211 is spaced apart from the second chamber 2222 and connected thereto via tubing 2280 and an aspiration valve 2422 in accordance with additional embodiments of the present technology. In the illustrated embodiment, the tubing 2480 provides a fluid path, via the valve 2481, from a first vacuum connection port 2482 of the first chamber 2211 to a second vacuum connection port 2483 of the second chamber 2212. The aspiration valve 2422 can be a gate valve, a stopcock valve, and/or another type of valve and can be movable/actuatable (e.g., via the control system 180 of FIG. 1) to provide a fluid path therethrough from the first chamber 2211 to the second chamber 2212. That is, the aspiration valve 2422 can be actuated to move between (i) an open position in which the first chamber 2211 is fluidly coupled to the second chamber 2212 (e.g., fluid is able to flow from the second chamber 2212 to the first chamber 2211) and (ii) a closed position in which the first chamber 2211 is fluidly decoupled to the second chamber 2212 (e.g., fluid is inhibited or even prevented from flowing from the second chamber 2212 to the first chamber 2211).

In the illustrated embodiment, the first chamber 2211 includes/comprises a first housing 2418 that includes the first vacuum connection port 2482 and the vacuum port 2216. The second chamber 2212 can include/comprise a second housing 2420 that includes the vent port 2213, the reinfusion port 2214, the positive pressure port 2217, and an aspiration port 2415. The aspiration port 2415 is fluidly coupled to the aspiration conduit 121, which is fluidly coupled to the aspiration lumen 104 of the aspiration catheter 102 as shown in FIG. 1. In some embodiments, the aspiration valve 122 (FIG. 1) can be omitted and the aspiration valve 2422 can provide the same or substantially similar functionality. Notably, compared to FIGS. 22 and 23, the aspiration port 2215 to the first chamber 2211 is omitted and, instead, the second chamber 2212 incorporates the aspiration port 2415 to the aspiration catheter 102 (FIG. 1).

In the illustrated embodiment, the filter plate 2230 and the valve assembly 2237 are mounted to a slanted wall 2470 within the second chamber 2212. These components separate the second chamber 2212 into an aspiration region 2485 and a collection or storage region 2486. The filter plate 2230 can be provided on a side of the filter plate 2230 facing the aspiration port 2415. In some embodiments, the aspiration port 2415 is provided in a sidewall of the second housing 2420 at a vertical level that is above or about equal to a vertical position of the filter plate 2230 and the valve assembly 2237.

Operation of the system 2200 is generally described with reference to FIGS. 1 and 24. Initially, the control system 180 can control the system 2200 to generate and store vacuum pressure in the first chamber 2211 by closing the aspiration valve 2422, closing the fluid inlet valve 152 and opening the vacuum valve 150. The pump 142 then operates to draw fluid (e.g., air) from the first chamber 2211 through the vacuum port 2216 and through the vacuum conduit 123 to generate vacuum in the first chamber 2211. The aspiration valve 2422 (and the valve assembly 2237) inhibits or even prevents vacuum generated in the first chamber 2211 from being applied to the second chamber 2222.

Next, the control system 180 can open the aspiration valve 2422 to fluidly connect the first chamber 2211 of the collection chamber 2210 to the aspiration lumen 104 of the aspiration catheter 102 via the aspiration conduit 121 such that at least a portion of the vacuum stored in the first chamber 2211 is applied to the aspiration lumen 104 of the aspiration catheter 102 to aspirate clot material and blood through the aspiration lumen 104 of the aspiration catheter 102, through the aspiration conduit 121, through the aspiration port 2215, and into the second chamber 2212. In some aspects of the present technology, a flow rate of the aspirated material can be relatively large, such as equal to or greater than about 30 cc/sec, greater than about 40 cc/sec, greater than about 50 cc/sec, greater than about 60 cc/sec, greater than about 70 cc/sec, greater than about 80 cc/sec, greater than about 90 cc/sec, greater than about 100 cc/sec, greater than about 120 cc/sec, greater than about 150 cc/sec, greater than about 200 cc/sec, greater than about 250 cc/sec, greater than about 300 cc/sec, greater than about 400 cc/sec, greater than about 500 cc/sec, or greater.

The high flow rate aspirated material can traverse laterally across the aspiration region 2485 of the second chamber 2212 and impact the filter plate 2230 and the valve assembly 2237. The filter plate 2230 is configured to filter out larger portions of the clot material while the impact of the aspirated material forcibly opens the valve assembly 2237 allowing blood and smaller portions of the clot material to pass through the valve assembly 2237 into the collection region 2486 of the second chamber 2212, as described in detail above with reference to FIGS. 23 and 24. The larger portions of the clot material that are filtered out can collect within the aspiration region 2485 of the second chamber 2212, while the blood and smaller portions of the clot material that pass through the valve assembly 2237 can collect within the collection region 2486 of the second chamber 2212.

The control system 180 can control the system 2200 to conduct one more additional aspiration cycles of charging vacuum within the first chamber 2211 and subsequently applying the charged vacuum to the aspiration catheter 102 to aspirate additional blood and clot material into the aspiration region 2485 of the second chamber 2212 and against the valve assembly 2237 at high flow rate, permitting the blood to pass into and collect in the collection region 2486 of the second chamber 2212. After each cycle (e.g., aspiration pass), more blood is stored in the collection region 2486 of the second chamber 2212. In some aspects of the present technology, the aspiration valve 2422 and/or the valve assembly 2237 inhibit or even prevent blood stored in the collection region 2486 of the second chamber 2212 from being exposed to vacuum when vacuum is generated in the first chamber 2211, which inhibits or even prevents hemolysis of the blood. In some embodiments, the first chamber 2211 can be maintained with vacuum pressure to provide a reservoir of stored vacuum that can be applied to the aspiration catheter 102 when the aspiration valve 2422 is opened. This can decrease the time required between subsequent aspiration passes because the first chamber 2211 need not be recharged with vacuum before a subsequent aspiration pass.

When an operator of the system 2200 (e.g., a physician) is ready to reinfuse blood collected in the collection region 2486 of the second chamber 2212 to the patient, the control system 180 can move the system 2200 to a reinfusion state by opening the positive pressure valve 151 and the reinfusion valve 126. The pump 142 then operates to pressurize the collection region 2486 of the second chamber 2212 via the positive pressure conduit 124 and the positive pressure port 2217 to drive/force the blood and the smaller portions of the clot material toward the filter 2233 and the reinfusion conduit 125 and into the patient.

Referring to FIGS. 22-24 together, in some embodiments the first chamber 2211 can include a vent port or positive pressure port that can be opened if some aspirated blood remains within the first chamber 2211 (FIGS. 22 and 23) or within the aspiration region 2285 of the second chamber 2212 (FIG. 24) after aspiration to reduce the vacuum level therein and inhibit or even prevent hemolysis of the blood. Moreover, in some aspects of the present technology the system 2200 can have significant orientational freedom as transfer of blood to the second chamber 2212 relies only on the flow rate of the aspirated material rather than gravity. That is, for example, referring to FIG. 24 the second chamber 2212 can be positioned above, below, or at the same vertical level as the first chamber 2211. Moreover, a position and/or orientation of the filter plate 2230 and the valve assembly 2237 can be selected to maximize impingement thereon of fluid aspirated through the aspiration port 2215/2415 to maximize or substantially maximize the amount of aspirated blood that passes into the collection region 2486 for storage.

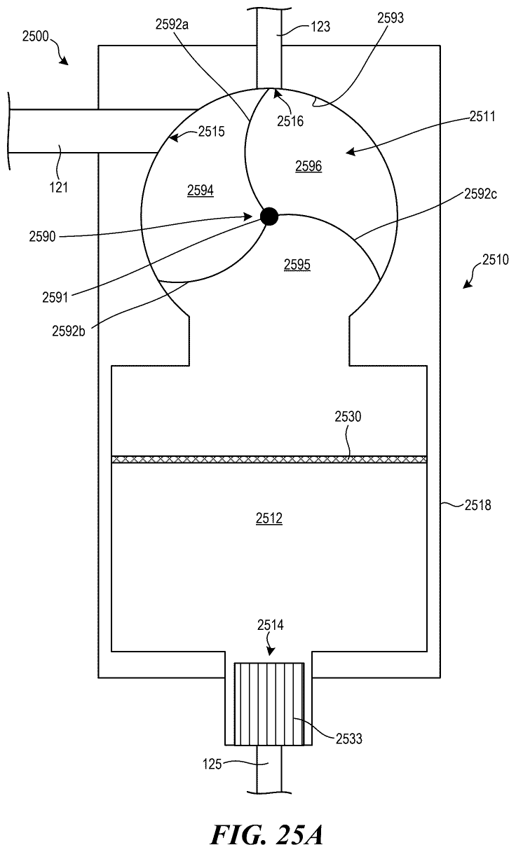

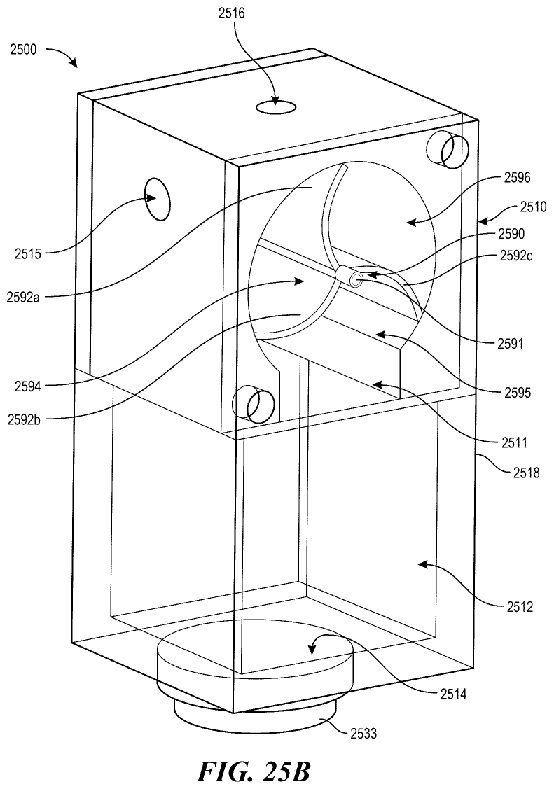

FIGS. 25A and 25B are a side cross-sectional view and a perspective view, respectively, of a portion of a clot treatment system 2500 (“system 2500”) including a collection chamber 2510 in accordance with additional embodiments of the present technology. The system 2500 can include several components generally similar or identical to, and can operate in a manner generally similar or identical to, any of the systems described in detail above with reference to FIGS. 1-24. For example, similar or identical components in FIGS. 25A and 25B are referred to with the same reference numbers as FIG. 1. Likewise, the system 2500 can include the control system 180 (not shown) described in detail above with reference to FIG. 1.

In the illustrated embodiment, the collection chamber 2510 includes a housing 2518 that encloses a first chamber 2511 and a second chamber 2512. In some embodiments, the second chamber 2512 can have a volume equal to or greater than about 100 cc, about 200 cc, about 300 cc, about 400 cc, about 500 cc, about 600 cc, about 1000 cc, greater than 1000 cc, and/or the like. The housing 2518 can include one or more openings/ports extending through the housing 2518 that provide fluid access to the first chamber 2511 and/or the second chamber 2512. For example, in the illustrated embodiment the housing 2518 includes an aspiration port 2515 to/from the first chamber 2511, a vacuum port 2516 to/from the first chamber 2511, and a reinfusion port 2514 to/from the second chamber 2512. In the illustrated embodiment, the aspiration port 2515 is fluidly coupled to the aspiration conduit 121 (FIG. 25A; omitted in FIG. 25B), which is fluidly coupled to the aspiration lumen 104 of the aspiration catheter 102 as shown in FIG. 1. The vacuum port 2516 can be fluidly coupled to the vacuum conduit 123, which is fluidly coupled to the pump assembly 140 as shown in FIG. 1. Likewise, the reinfusion port 2514 can be fluidly coupled to the reinfusion conduit 125, which is fluidly coupled to the reinfusion lumen 108 of the reinfusion catheter 106 as shown in FIG. 1. In some embodiments, the housing 2518 can further include a vent port (not shown) to provide a vent to atmosphere for the second chamber 2512 and/or a positive pressure port (not shown) to/from the second chamber 2512 to allow pressurization of the second chamber 2512 to, for example, drive aspirated blood through the reinfusion port 2514.

Referring to FIG. 25A, the collection chamber 2510 can further include a filter tray 2530 spanning laterally across the housing 2518, and a filter 2533 fluidly coupled between the reinfusion port 2514 and the reinfusion conduit 125. In some embodiments, the filter 2533 defines the reinfusion port 2514. As described in detail above with reference to FIG. 1, the filter tray 2530 can have a first porosity and the filter 2533 can have a second porosity less than the first porosity.

Referring to FIGS. 25A and 25B, in the illustrated embodiment the collection chamber 2510 includes a vane member 2590 rotatably mounted in the first chamber 2511. The vane member 2590 can include a hub 2591 and a plurality of spokes 2592 (including individually identified first through third spokes 2592a-c, respectively) extending radially outward from the hub 2591. The spokes 2592 can each sealingly engage an inner surface 2593 of the first chamber 2511. In some embodiments, the spokes 2592 include one or more sealing members coupled to tips thereof (e.g., tips radial outward of the hub 2591) to facilitate sealing engagement with the inner surface 2593. The hub 2591 can be connected to a motor (e.g., a servomotor) or another component configured to rotate the vane member 2590 within the first chamber 2511. The motor can be controlled by the control system 180 of FIG. 1. In the illustrated embodiment, the first and second spokes 2592a-b define a first chamber region 2594 therebetween, the second and third spokes 2592b-c define a second chamber region 2595 therebetween, and the third and first spokes 2592c, a define a third chamber region 2596 therebetween.

Referring to FIG. 25A, in the illustrated embodiment the vane member 2590 is rotatably positioned within the first chamber 2511 in an initial position such that the first chamber region 2594 and the third chamber region 2596 are fluidly connected to the vacuum port 2516 and can therefore both have vacuum pressure generated therein. In contrast, the second chamber region 2595 is fluidly connected to the second chamber 2512 (which may be vented) and is thus not under vacuum. Additionally, the first chamber region 2594 is also fluidly connected to the aspiration port 2515.

Operation of the system 2500 is generally described with reference to FIGS. 1 and 25A. Initially, with the vane member 2590 in the initial state shown in FIG. 25A, the control system 180 can control the system 2500 to generate and store vacuum pressure in the first chamber region 2594 and the third chamber region 2596 by closing the aspiration valve 122, closing the fluid inlet valve 152, and opening the vacuum valve 150. The pump 142 then operates to draw fluid (e.g., air) from the first chamber region 2594 and the third chamber region 2596 through the vacuum port 2216 and through the vacuum conduit 123 to generate vacuum in the first chamber region 2594 and the third chamber region 2596. The sealing engagement of the second and third spokes 2592b, c with the inner surface 2593 of the first chamber 2511 inhibits or even prevents vacuum from being applied to the second chamber 2212.

Next, the control system 180 can open the aspiration valve 122 to fluidly connect the first chamber region 2594 to the aspiration lumen 104 of the aspiration catheter 102 via the aspiration conduit 121 such that at least a portion of the vacuum stored in the first chamber region 2594 is applied to the aspiration lumen 104 of the aspiration catheter 102 to aspirate clot material and blood through the aspiration lumen 104 of the aspiration catheter 102, through the aspiration conduit 121, through the aspiration port 2515, and into the first chamber region 2594.

Once the aspiration is completed (e.g., substantially all the vacuum stored in the first chamber region 2594 is applied to the aspiration catheter 102), the control system 180 can control the motor to rotate the vane member 2590 in a counterclockwise direction to increment the positions of the first chamber region 2594, the second chamber region 2595, and the third chamber region 2596 (collectively “chamber regions 2594-2596”) such that the first chamber region 2594 takes the place of the second chamber region 2595, the second chamber region 2595 takes the place of the third chamber region 2596, and the third chamber region 2596 takes the place of the first chamber region 2594. This movement fluidly couples the first chamber region 2594 to the second chamber 2512 to allow the clot material and blood aspirated into the first chamber region 2594 to fall into the second chamber 2512 via gravity, where the filter tray 2530 can filter out larger portions of the clot material. In some aspects of the present technology, quickly rotating the first chamber region 2594 to be fluidly connected to the second chamber 2512 after aspiration minimizes the time the aspirated blood is subject to vacuum pressure—inhibiting or even preventing hemolysis thereof.

Additionally, after rotation of the vane member 2590, the third chamber region 2596 is fluidly connected to the aspiration port 2515 such that the vacuum stored therein can be immediately applied to the aspiration catheter 102 to aspirate additional blood and clot material into the third chamber region 2596. In some aspects of the present technology, this can minimize the time required to perform sequential aspiration passes. In particular, there is little or no wait time between aspiration into the first chamber region 2594 and aspiration into the third chamber region 2596 after rotation of the vane member 2590. Alternatively, before and/or during rotation of the vane member 2590, the control system 180 can close the aspiration valve 122 such that the vacuum stored in the third chamber region 2596 is not immediately applied to the aspiration catheter 102. Subsequent opening of the aspiration valve 122 can apply the vacuum stored in the third chamber region 2596 to the aspiration catheter 102 to aspirate additional blood and clot material therein.

Likewise, after rotation of the vane member 2590, the second chamber region 2595 is fluidly coupled to the vacuum port 2516. In this manner, vacuum can be pre-charged within the second chamber region 2595 via the vacuum port 2516 as, for example, material is aspirated into the third chamber region 2596. The control system 180 can continue to increment the vane member 2590 to rotate the chamber regions 2594-2596 between positions to provide multiple aspiration passes and to move the aspirated material, including blood, to the second chamber 2512 for storage at ambient or other non-vacuum pressure, thereby inhibiting or even preventing hemolysis of the blood.

When an operator of the system 2500 (e.g., a physician) is ready to reinfuse blood collected in the second chamber 2512 to the patient, a syringe or other pressure source can be coupled to the reinfusion conduit 125 and used to draw blood through the filter 2533 for subsequent reinfusion to the patient. In some embodiments, the housing 2518 includes a positive pressure port (not shown), and the control system 180 can move the system 2500 to a reinfusion state by opening the positive pressure valve 151 and the reinfusion valve 126. The pump 142 then operates to pressurize second chamber 2512 via the positive pressure conduit 124 and the positive pressure port to drive/force the blood and the smaller portions of the clot material toward the filter 2533 and the reinfusion conduit 125 and into the patient.

Referring to FIGS. 11-25B together, the various systems could rest on a table or other stationary surface, hang on a stand/pole (e.g., an intravenous stand/pole), or be handheld by the user for operation. Additionally, some or all of the various ports can be combined/omitted. For example, the positive pressure port and the vacuum port could be combined into a single port, there could be a positive pressure port to both the first/upper chamber and to the second/lower chamber, and so on.

Additionally, the various systems can be configured to charge vacuum within a vacuum chamber to a level that decays upon aspiration to inhibit or even prevent hemolysis of blood. For example, a vacuum chamber can be charged to a preset vacuum level, such as 25 inches of mercury (inHg) or 28 inHg, before a vacuum inlet to the vacuum chamber is closed by a valve or other mechanism. When the aspiration valve is opened to aspirate clot material and blood into the vacuum chamber, the vacuum will decay and approach 0 inHg when the chamber is fully filled with the aspirated clot material and blood. Because the level of vacuum in the vacuum chamber decays, the blood is not held at high vacuum while in the vacuum chamber and therefore prevents hemolysis of the blood. The blood can then be transferred to another chamber, a syringe, and/or directly to the patient as described in detail above.

Moreover, any of the various systems can include mechanisms to control the temperature of aspirated blood to reduce hemolysis. Specifically, reducing the temperature to chill the blood can improve the stability of the lipid bilayer of the red blood cells. Temperature control zones can be placed on various tubing components, in the collection chamber, in any secondary chambers, and/or on the tubing for blood reintroduction. Before reintroduction of blood, the blood may be reheated to body temperature to place it in homeostasis with the body. Thermoelectric cooling, one or more heat pumps, circulation of a cryogenic fluid, and/or other cooling components can be used to provide blood cooling. Thermoresistive heating elements and/or other heating elements can be used to provide blood heating before reintroduction of the blood to the patient. The temperature of the blood can be reduced to less than about 37° Celsius (C), but above about 0° C. The blood can be reheated for introduction to the patient to at least 32° C., and ideally to about 37° C.

### IV. SELECTED EMBODIMENTS OF ADDITIONAL CLOT FILTRATION DEVICES

In some embodiments, a clot treatment system in accordance with the present technology can include one or features configured to filter clot material from blood. As described in detail above, a clot treatment system can include a coarse filter (e.g., filter tray) to provide first-stage filtration of larger portions of clot material from blood, and one or more finer filters configured to provide second-stage filtration of smaller portions of the clot material from the blood such that the blood is suitable for reintroduction to the patient. The combination of coarse and fine filters can help inhibit or even prevent clogging of the clot treatment system during operation. In some embodiments, the arrangement/geometry of coarse and fine filters can be optimized to minimize the risk of clogging and extend the life of the filters.

FIG. 26, for example, is a side cross-sectional view of a portion of a clot treatment system 2600 (“system 2600”) including a collection chamber 2610 in accordance with additional embodiments of the present technology. The system 2600 can include several components generally similar or identical to, and can operate in a manner generally similar or identical to, any of the systems described in detail above with reference to FIGS. 1-25B. For example, similar or identical components in FIG. 26 are referred to with the same reference numbers as FIG. 1. Likewise, the system 2600 can include the control system 180 (not shown) described in detail above with reference to FIG. 1.

In the illustrated embodiment, the collection chamber 2610 includes a housing 2618 that encloses a chamber 2611. The housing 2618 can include one or more openings/ports extending through the housing 2618 that provide fluid access to the chamber 2611. For example, in the illustrated embodiment the housing 2618 includes an aspiration port 2615 to/from the chamber 2611, a vacuum port 2616 to/from the chamber 2611, a positive pressure port 2617 to/from the chamber 2611, and a reinfusion port 2614 to/from the chamber 2611. In the illustrated embodiment, the aspiration port 2615 is fluidly coupled to the aspiration conduit 121, which is fluidly coupled to the aspiration lumen 104 of the aspiration catheter 102 as shown in FIG. 1. The vacuum port 2616 can be fluidly coupled to the vacuum conduit 123, which is fluidly coupled to the pump assembly 140 as shown in FIG. 1. The positive pressure port 2617 can be fluidly coupled to the positive pressure conduit 124, which is fluidly coupled to the pump assembly 140 as shown in FIG. 1. Likewise, the reinfusion port 2514 can be fluidly coupled to the reinfusion conduit 125, which is fluidly coupled to the reinfusion lumen 108 of the reinfusion catheter 106 as shown in FIG. 1. In some embodiments, the housing 2618 can further include a vent port (not shown) to provide a vent to atmosphere for the chamber 2611.

In the illustrated embodiment, the collection chamber 2510 can further include a first coarse filter 2690 (e.g., a first filter tray, a first gross filter), a second coarse filter 2691 (e.g., a second filter tray, a second gross filter) positioned within the housing 2618, and a filter 2633 fluidly coupled between the reinfusion port 2614 and the reinfusion conduit 125. In some embodiments, the first coarse filter 2690 and the second coarse filter 2691 each have a porosity greater than a porosity of the filter 2633 such that they are configured to filter out larger portions of clot material aspirated into the chamber 2611 via the aspiration port 2615 and the aspiration conduit 121, while the filter 2633 is configured to filter out smaller portions of the clot material such that the blood is suitable for reinfusion into a patient.

In some embodiments, the first coarse filter 2690 and the second coarse filter 2691 each have a three-dimensional shape. For example, the first coarse filter 2690 and the second coarse filter 2691 can each have an inverted conical shape as shown in FIG. 26, or can have a cylindrical or other shape. In the illustrated embodiment, the first coarse filter 2690 and the second coarse filter 2691 each span across and filter 2633 to inhibit larger portions of clot material from reaching the filter 2633. In some aspects of the present technology, the conical shapes of the first coarse filter 2690 and the second coarse filter 2691 can enable clot material to slide down an outer (e.g., upper) surface thereof while allowing fluid to pass through the pores thereof. The first coarse filter 2690 and the second coarse filter 2691 can have the same or different porosities. For example, the first coarse filter 2690 can have a larger porosity than the second coarse filter 2691 (e.g., the first coarse filter 2690 has larger pores than pores of the second coarse filter 2691 to permit larger particles to pass therethrough). The pores of the first coarse filter 2690 and/or the second coarse filter 2691 can have any number of shapes, such as circular, slits (e.g., rectangular), etc. In some aspects of the present technology, the three-dimensional (e.g., conical) shape of the first coarse filter 2690 and the second coarse filter 2691 and the inclusion of multiple coarse filters can inhibit clogging of the collection chamber 2610 during operation of the system 2600. In some embodiments, an angle of the conically-shaped first coarse filter 2690 and the second coarse filter 2691 can be tuned to optimize the maximum amount of fluid passing through the pores, as well as to allocate space for discarded (e.g., filtered out) clot material on a periphery thereof without inhibiting or even preventing further filtering during subsequent operation of the system 2600.

In some embodiments, the collection chamber 2610 can include more than two of the coarse filters (e.g., three or more). Likewise, one or more of the coarse filters can have different shapes. For example, one or both of the first coarse filter 2690 and the second coarse filter 2691 can have a two-dimensional shape (e.g., a flat plane, an angled plane, a curved plane, etc.) For example, one or both of the first coarse filter 2690 and the second coarse filter 2691 can be flat planar, like the filter tray 130 of FIG. 1. The first coarse filter 2690 and the second coarse filter 2691 can be removable or replaceable from within the collection chamber 2610 such that any clogging material can be removed if the filtering rate of the collection chamber 2610 diminishes during operation. The first coarse filter 2690 and the second coarse filter 2691 can further include features to ensure that clot remains retained thereupon upon removal from the chamber 2611. Although the first coarse filter 2690 and the second coarse filter 2691 are positioned within the chamber 2611, in other embodiments, one or more coarse filters may be located in a vented chamber, along a length of tubing of the system 2600, etc. For example, a coarse filter could be positioned within the aspiration conduit 121.

In the illustrated embodiment, the housing 2618 includes a lower collection surface 2619 having an angled (e.g., conical) shape. This shape of the lower collection surface 2619 can help direct fluid toward a center of the chamber 2611 toward the filter 2633 and thus provide a more direct path for the fluid to the filter 2633 to further inhibit clogging and facilitate transfer of fluid (e.g., blood) through the filter 2633.

In some embodiments, a porosity/pore size of the first coarse filter 2690 and/or the second coarse filter 2691 can be heterogenous instead of homogenous. FIG. 27, for example, is a side view of a conical coarse filter 2790 in accordance with embodiments of the present technology. In the illustrated embodiment, the coarse filter 2790 includes a base 2793, an apex 2794 opposite the base 2793, and a plurality of pores 2795 extending therethrough. The pores 2795 can change (e.g., increase) in size in a direction indicated by the arrow A from the apex 2794 toward the base 2793. In some aspects of the present technology, this can allow for seepage of additional fluid (e.g., blood) through the coarse filter 2790 near the base 2793 where filtered larger portions of clot material may collect after sliding down an outer surface 2796 of the coarse filter 2790.

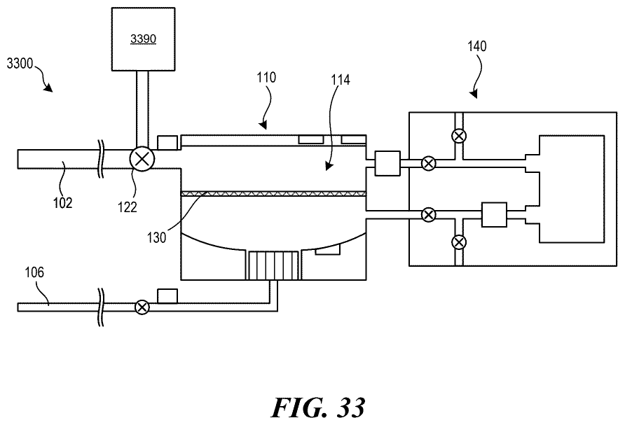

FIG. 33 is a side cross-sectional view of a clot treatment system 3300 (“the system 3300”) in accordance with additional embodiments of the present technology. The system 3300 can include several components generally similar or identical to, and can operate in a manner generally similar or identical to, any of the systems described in detail above with reference to FIGS. 1-32. For example, similar or identical components in FIG. 33 are referred to with the same reference numbers as in FIG. 1. Likewise, the system 3300 can include the control system 180 (not shown) described in detail above with reference to FIG. 1.

In the illustrated embodiment, the aspiration valve 122 is fluidly coupled to a fluid reservoir 3390 filled with saline or another fluid. In the illustrated embodiment, the aspiration valve 122 can be actuated to move between (i) a first position in which the aspiration catheter 102 is fluidly coupled to the collection chamber 110 and the fluid reservoir 3390 is fluidly decoupled from the collection chamber 110 (e.g., fluid is inhibited or even prevented from flowing from the fluid reservoir 3390 to the collection chamber 114) and (ii) a second position in which the aspiration catheter 102 is fluidly decoupled from the collection chamber 110 and the fluid reservoir 3390 is fluidly coupled to the collection chamber 110 (e.g., fluid is able to flow from the fluid reservoir 3390 to the collection chamber 114). Accordingly, the aspiration valve 122 can be a three-way stopcock or another type of valve. In some embodiments, the aspiration valve can be actuated (e.g., electromechanically by the control system 180 of FIG. 1) to have a third position in which each of the collection chamber 110, the aspiration catheter 102, and the fluid reservoir is fluidly decoupled from one another.

In other embodiments, the fluid reservoir 3390 can be fluidly coupled to the aspiration conduit 121 (e.g., adjacent to the aspiration valve 122) via a second aspiration valve. For example, the aspiration valve 122 can be operated as described above in detail with reference to FIG. 1, and the second aspiration valve can be fluidly coupled to the fluid reservoir 3390 to control flow from the fluid reservoir 3390 into the collection chamber 110. More specifically, the second aspiration valve can be actuated to move between (i) an open position in which the fluid reservoir 3390 is fluidly coupled to the collection chamber 110 and (ii) a closed position in which the fluid reservoir 3390 is fluidly decoupled from the collection chamber 110.

In some aspects of the present technology, the fluid reservoir 3390 can be used to flush and/or clear the system 3300 after aspiration and/or reinfusion. For example, after reinfusion, the aspiration valve 122 can be moved to the second position to fluidly connect the fluid reservoir 3390 to the collection chamber 110, and saline can be pulled via the pump assembly 140 into the collection chamber 110 and subsequently driven into the reinfusion catheter 106 via the pump assembly 140, similar to the process described in greater detail above with reference to aspiration of the aspiration catheter 102 in FIGS. 5A-5H, to flush and remove residual filtered blood from inside components (e.g., the collection chamber 110) of the system 3300. The saline and residual filtered blood can then be reinfused into the patient to increase the quantity of blood that is reinfused into the patient during the operation of the system 3300. Additionally, flushing the system 3300 can enhance the user's view of clot material (e.g., the clot material 590 shown in FIG. 5E) on the filter tray 130, allowing them to assess whether additional aspiration is appropriate. Further, flushing the system 3300 can enhance the user's view of fluid levels within the collection chamber 110, allowing them to assess whether reinfusion or additional aspiration is appropriate. In other embodiments, the saline can flow at least partially through the system 3300 under the influence of gravity (e.g., gravity-driven into the collection chamber 110).

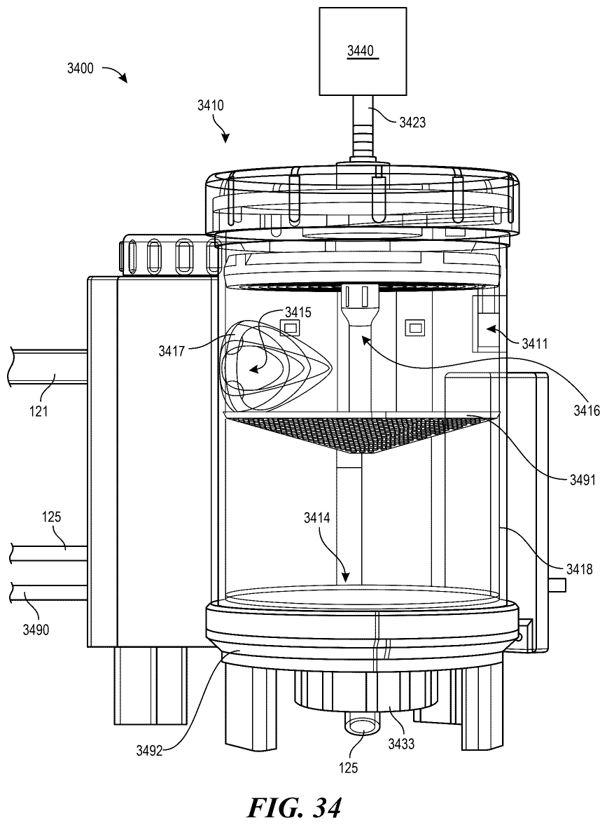

FIG. 34 is a partially-schematic side view of a portion of a clot treatment system 3400 (“the system 3400”) in accordance with additional embodiments of the present technology. The system 3400 can include several components generally similar or identical to, and/or can operate in a manner generally similar or identical to, the systems 1100, 1800, 2100, 2200, 2500, 2600 described in detail above with reference to FIGS. 11-13 and 18-26. For example, similar or identical components in FIGS. 34-41F are referred to with the same reference numbers as FIG. 11. Likewise, the system 3400 can include the control system 180 (not shown) described in detail above with reference to FIGS. 1 and 11-13.

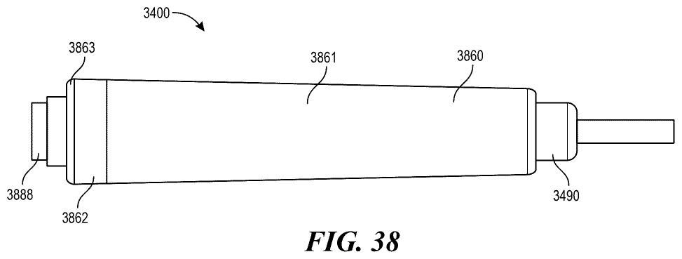

In the illustrated embodiment, the system 3400 includes a collection chamber 3410 having a housing 3418 that encloses a chamber 3411. The housing 3418 can include one or more openings/ports extending through the housing 3418 that provide fluid access to the chamber 3411. For example, in the illustrated embodiment, the housing 3418 includes an aspiration port 3415 to/from the chamber 3411, a vacuum port 3416 to/from the chamber 3411, and a reinfusion port 3414 to/from the chamber 3411. In the illustrated embodiment, the aspiration port 3415 is fluidly coupled to the aspiration conduit 121, which is fluidly coupled to the aspiration lumen 104 of the aspiration catheter 102 as shown in FIG. 1. The vacuum port 3416 can be fluidly coupled to a pressure conduit 3423, which is fluidly coupled to a pump assembly 3440 configured to generate vacuum pressure and positive pressure in the chamber 3411, as described in greater detail below. Likewise, the reinfusion port 3414 can be fluidly coupled to the reinfusion conduit 125, which is fluidly coupled to the reinfusion lumen 108 of the reinfusion catheter 106 as shown in FIG. 1. In some embodiments, the housing 3418 can further include a vent port (not shown) to provide a vent to atmosphere for the chamber 3411. In the illustrated embodiment, the collection chamber 3410 further includes a first coarse filter 3491 (e.g., a first filter tray, a first gross filter) and a filter 3433 fluidly coupled between the reinfusion port 3414 and the reinfusion conduit 125. Additionally, the housing 3418 is coupled to a remote cable 3490. The remote cable 3490 can be electrically or operably coupled to the control system 180 (not shown) or an actuator (e.g., remote 3860 (FIG. 38)) to control fluid flow within the system 3400.

In the illustrated embodiment, the pressure conduit 3423 is a combination of the vacuum conduit 123 and the positive pressure conduit 124 of FIG. 1. More specifically, the pump assembly 3440 can generate negative and positive pressure within a chamber 3411 via the pressure conduit 3423. In some embodiments, the pump assembly 3440 is a generic pump that can be removably attached to various equipment within a hospital. In some aspects of the present technology, this configuration can make the system 3400 compatible with preexisting pumps, reducing the cost of the system 3400.

In the illustrated embodiment, the aspiration port 3415 includes curved surfaces 3417 that guide aspirated material around an inside surface of the chamber 3411 during aspiration. More specifically, aspirated material flows from the aspiration conduit 121 into the aspiration port 3415 at an angle relative to the chamber 3411 (e.g., tangentially) such that the aspirated material is directed toward the curved surfaces 3417 rather than a center of the chamber 3411. Accordingly, the curved surfaces 3417 will direct the aspirated material at least partially around an inner surface of the chamber 3411 in a generally helical or swirling motion before the aspirated material settles onto the first coarse filter 3491. More specifically, rather than travelling linearly through the chamber 3411 at a high flow rate during aspiration and hitting the chamber 3411 opposite the aspiration port 3415, the aspirated material will circle around the chamber 3411 and slow until it settles onto the first coarse filter 3491 or into other aspirated material. In some aspects of the present technology, this configuration can reduce the force (e.g., turbulence, shearing forces) applied to the aspirated material, which can inhibit or even prevent hemolysis in the aspirated blood. In some embodiments, the curved surfaces extend around at least a portion of a perimeter of the chamber 3411 to further direct the flow of the aspirated material. In other embodiments, a diameter of the aspiration conduit 121 can gradually increase proximally near the chamber 3411 to slow the flow of the aspirated material before it reaches the chamber 3411, which can likewise reduce the risk of hemolysis in the aspirated blood.

In the illustrated embodiment, the housing 3418 includes an LED strip 3492 positioned around a bottom perimeter of the housing 3418. In other embodiments, the LED strip 3492 can be positioned at other locations on the housing 3418. In some embodiments, the LED strip 3492 is electrically coupled to the control system 180 and can receive electrical signals from the control system 180 that determine the color, intensity, pattern, and/or the like of the LED strip 3492. In some aspects of the present technology, the LED strip 3492 can be illuminated to provide feedback to the user regarding the status of the system 3400, such as a state of the system (e.g., aspiration, reinfusion, blood level in the chamber 3411, error or warning, etc.). In some embodiments, inner surfaces of the chamber 3411 can be illuminated by the LED strip 3492 or other LEDs positioned adjacent to the chamber 3411 to enhance visibility of the clot material (e.g., clot material 590 (FIG. 5E)).

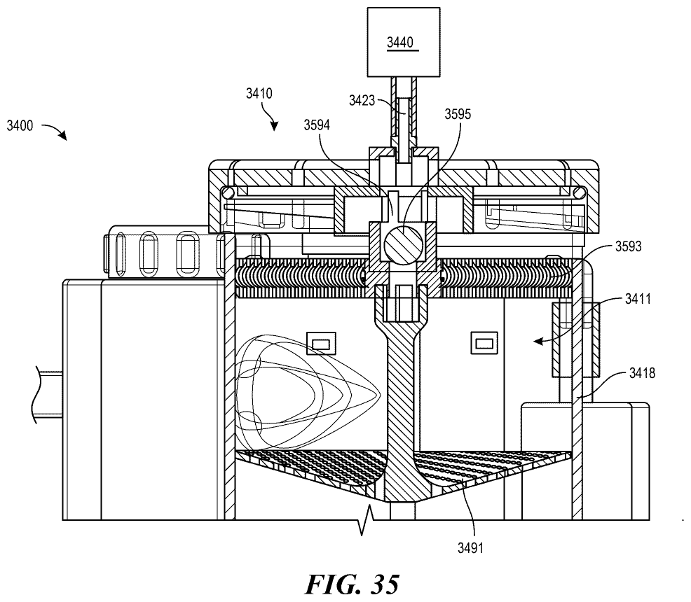

FIG. 35 is an enlarged, partially-schematic side view of the portion of the clot treatment system 3400 of FIG. 34 in accordance with additional embodiments of the present technology. In the illustrated embodiment, the collection chamber 3410 includes a defoamer 3593 positioned in the chamber 3411. The defoamer 3593 can inhibit bubbles in the aspirated material from rising toward the pump assembly 3440 by popping bubbles that come in contact with the defoamer 3593. In some aspects of the present technology, this can inhibit blood and/or clot material from rising in the chamber 3411 and inadvertently entering (e.g., being sucked into) the pump assembly 3440.

In the illustrated embodiment, the housing 3418 further includes a baffle 3594 and a float ball 3595. While the system is 3400 is generally not intended to aspirate sufficient blood and clot material to fill the chamber 3411 sufficiently high to reach the baffle 3594, the baffle 3594 and ball 3595 can act as a backup shut-off to the aspiration line. For example, if blood reaches the baffle 3594 and the float ball 3595, the float ball 3595 will float along a top surface of the aspirated material due to its buoyancy, in line with the baffle 3594 (e.g., along a fixed path). More specifically, as the level of the aspirated material within the chamber 3411 increases (e.g., rises), the float ball 3595 rises upwards toward the baffle 3594. Once the float ball 3595 reaches the baffle 3594 (e.g., the chamber 3411 is sufficiently full of aspirated material), the float ball 3595 substantially fills the baffle 3594 to block flow to and/or from the pressure conduit 3423. That is, the float ball 3595 fluidly disconnects the pump assembly 3440 from the chamber 3411. In some aspects of the present technology, this inhibits or even prevents blood and/or clot material from being inadvertently sucked into the pump assembly 3440 when the level of aspirated material within the chamber 3411 rises. In some embodiments, when the baffle 3594 is blocked by the float ball 3595, the pump assembly 3440 can be actuated to (i) exert positive pressure on the float ball 3595, pushing it downward and away from the baffle 3594 to reopen flow from the pressure conduit 3423 to the chamber 3411, and to (ii) reinfuse the filtered blood and decrease the level of aspirated material within the chamber 3411. In other embodiments, the user can wait for the aspirated material to naturally filter through the first coarse filter 3491 and for the level of aspirated material to decrease enough for the baffle 3594 to be unblocked by the float ball 3595 before performing subsequent operations. In some embodiments, either the defoamer 3593 or the baffle 3594 and float ball 3595 can be used, or both can be used in combination to inhibit blood and/or clot material from entering the pump assembly 3440.

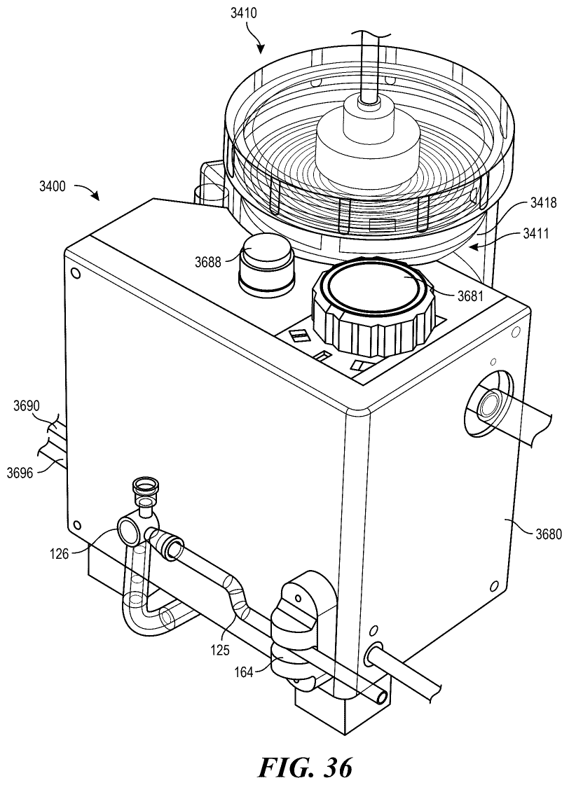

FIG. 36 is a perspective rear view of the portion of the clot treatment system 3400 of FIG. 34 in accordance with additional embodiments of the present technology. In the illustrated embodiment, the chamber 3411 is fluidly coupled to a flush line 3696. The flush line 3696 can be fluidly coupled to a fluid reservoir (e.g., fluid reservoir 3390 (FIG. 33)) that houses fluid for flushing out the collection chamber 3410, as described in detail above with reference to FIG. 33.

In the illustrated embodiment, the housing 3418 of the collection chamber 3410 is coupled to an electronics housing 3680. In some embodiments, the electronics housing 3680 includes electrical components that power and/or control the system 3400, such as the control system 180 of FIG. 1. In further embodiments, the electronics housing 3680 can be removably coupled to the housing 3418 such that the collection chamber 3410 can be replaced. For example, a first collection chamber removably coupled to the electronics housing 3680 can be used in a first procedure, removed and disposed of, and a second collection chamber can be coupled to the electronics housing 3680 for a second procedure. In other embodiments, other components of the housing 3418 and/or the electronics housing 3680 can be interchangeable to make the system 3400 reusable. The system 3400 can also have a power cable 3690 configured to be coupled to an external power source (e.g., AC wall power) to power some or all of the components of the system 3400.

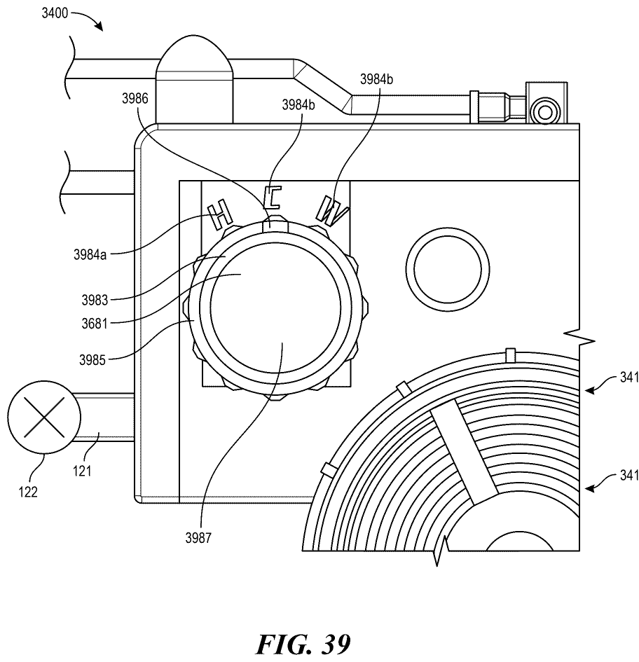

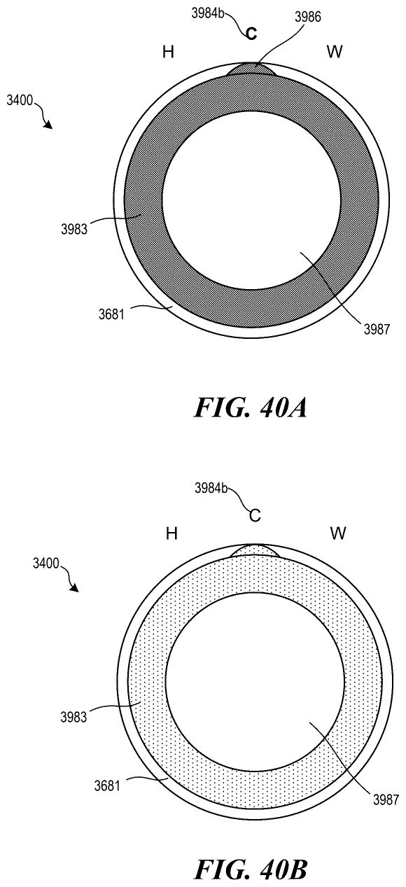

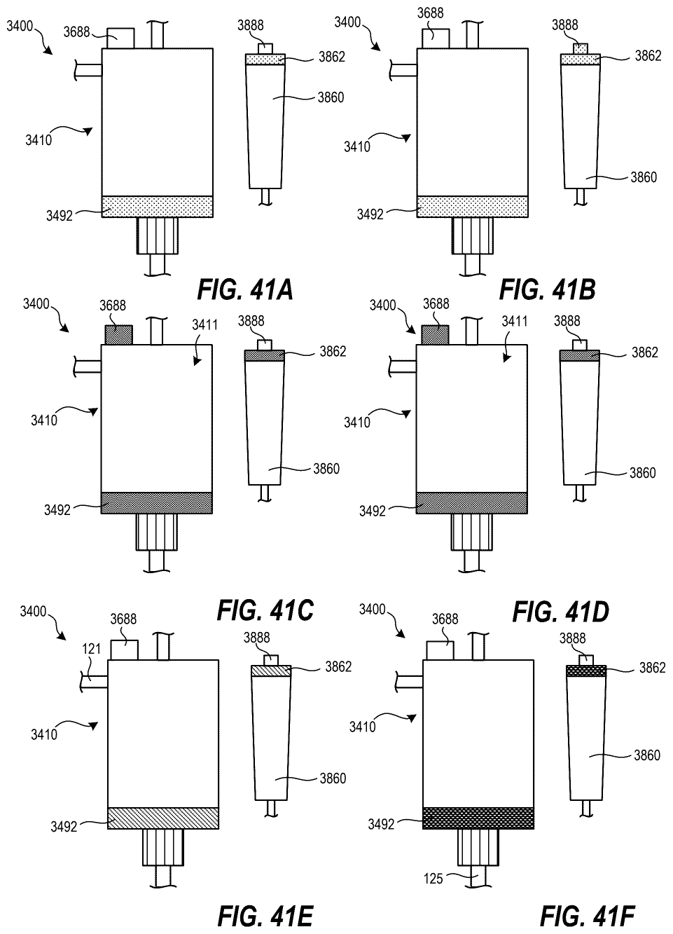

In the illustrated embodiment, the bubble sensor 164 of FIG. 1 is coupled to the electronics housing 3680 and is positioned along the reinfusion conduit 125 between the reinfusion catheter 106 (FIG. 1) and the reinfusion valve 126. The electronics housing 3680 further includes a button 3688 and a mode selector 3681 operably coupled to a control system (e.g., the control system 180 (FIG. 1)). The button 3688 can be actuatable by a user to, for example, cause the control system 180 to initiate one or more filtered blood reinfusion operations, as described in greater detail above with reference to FIG. 3 and below with reference to FIGS. 41C and 41D. The mode selector 3681 can be actuatable by a user to select between different modes of operation, such as, for example, hold mode, continuous mode, volume-specified mode, pulse mode, and/or the like, as described in greater detail below with reference to FIGS. 39-40B.

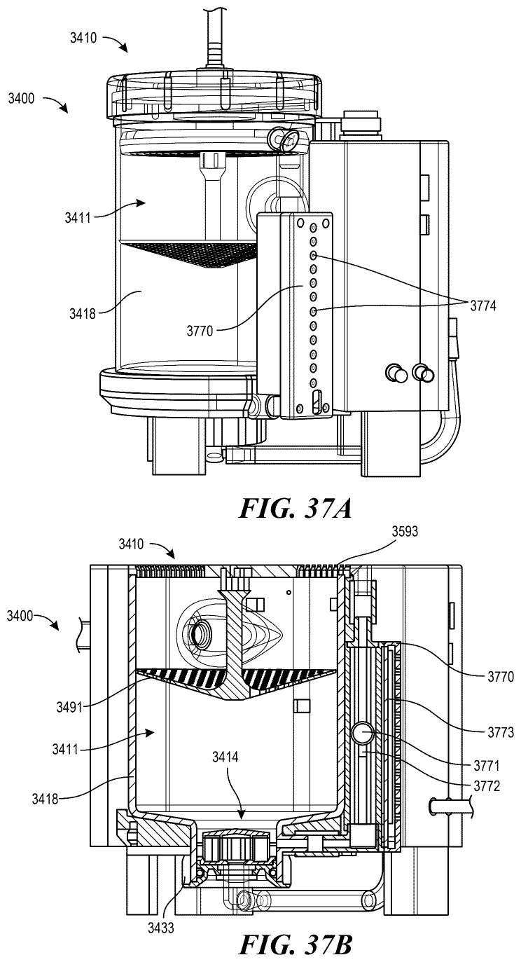

FIGS. 37A and 37B are side views of the portion of the clot treatment system 3400 of FIG. 34 in accordance with additional embodiments of the present technology. Referring to FIGS. 37A and 37B together, in the illustrated embodiment the housing 3418 of the collection chamber 3410 includes a fluid level detector 3770 electrically coupled to the control system 180 (FIG. 1) that detects the level of fluid within the chamber 3411. Referring to FIG. 37A, the fluid level detector 3770 can include one or more features 3774 on an external surface of the fluid level detector 3770 that indicate to the user the level of fluid within the chamber 3411, such as windows showing a location of a float ball 3771, LEDs, etc.

Referring to FIG. 37B, the fluid level detector 3770 includes the float ball 3771, a magnet 3772 coupled to the float ball 3771, and a hall effect printed circuit board (PCB) 3773. The float ball 3771 floats along a top surface of the aspirated material due to its buoyancy, carrying the magnet 3772, and the hall effect PCB 3773 can detect the proximity of the magnet 3772 as it moves within the fluid level detector 3770. More specifically, as the level of the aspirated material within the chamber 3411 increases (e.g., rises), the float ball 3771 rises upward. Movement of the float ball 3771 is sensed by the hall effect PCB 3773 due to the attached magnet 3772, allowing the level of aspirated material within the chamber 3411 to be measured. The control system 180 (FIG. 1) can receive the fluid level measurements from the fluid level detector 3770. In some embodiments, when the level of aspirated material is measured to be low (e.g., close to the reinfusion port 3414), the control system 180 can control the pump assembly 3440 (FIG. 34) to cease or prohibit generation of positive pressure in the chamber 3411 (e.g., by shutting of the pump assembly 3440, or closing the vacuum valve 3416 of FIG. 34) to inhibit or even prevent air from being reinfused into the patient. In other embodiments, when the level of aspirated material is measured to be high (e.g., close to the first coarse filter 3491, close to the defoamer 3593), the control system 180 can cease or prohibit generation of negative pressure in the chamber 3411 (e.g., by shutting of the pump assembly 3440, or closing the vacuum valve 3416 of FIG. 34) and/or can prompt the user to initiate a reinfusion operation.

FIG. 38 is a side view of a remote 3860 that can be utilized in the system 3400 of FIG. 34 and/or other systems described herein in accordance with embodiments of the present technology. In the illustrated embodiment, the remote 3860 is electrically coupled to the electronics housing 3680 (FIG. 36) via the remote cable 3490 and can be used to control fluid flow within the system 3400—for example, aspiration and reinfusion operations. In some embodiments, the remote 3860 is operably coupled to the control system 180 (FIG. 1). In the illustrated embodiment, the remote 3860 includes a body 3861 having an LED strip 3862 positioned circumferentially around a longitudinal axis of the body 3861 near an end 3863 of the body 3861. In some embodiments, the LED strip 3862 can be positioned at other locations on the remote 3860, such as adjacent to the remote cable 3490. In the illustrated embodiment, the remote 3860 further includes a button 3888 at the end 3863 of the body 3861 opposite the remote cable 3490. The button 3888 can be actuated to control the operation of the system 3400. For example, the button 3888 can be depressed to aspirate the aspiration catheter 102 (FIG. 1), as described in greater detail above. In some embodiments, the LED strip 3862 and/or the button 3888 can be illuminated based on the status of the system 3400, as described in greater detail below with reference to FIGS. 41A-41F. In some embodiments, the remote 3860 can include a vibration device that causes the remote 3860 to vibrate, providing feedback to the user about the status of the system 3400.

FIG. 39 is a partially-schematic top view of the portion of the clot treatment system 3400 of FIG. 34 in accordance with additional embodiments of the present technology. In the illustrated embodiment, the mode selector 3681 includes a recessed light 3983 that can be illuminated based on the status of the system 3400, as described in greater detail below with reference to FIGS. 40A and 40B. The mode selector 3681 includes three different modes 3984 (individually labeled first mode 3984a, second mode 3984b, and third mode 3984c). The different modes 3984 can be individually selected by turning a rotating wheel 3985 of the mode selector 3681. More specifically, the rotating wheel 3985 can be rotated such that a selection indicator 3986 is aligned with the desired mode 3984. In other embodiments, the mode selector 3681 can include more or fewer modes, depending on the intended operation of the system 3400. For example, when the system 3400 is implemented in more delicate vasculature, more aggressive modes can be removed to reduce the risk of accidental damage to the vasculature.

Referring to FIGS. 38 and 39 together, which of the modes 3984 is selected can control the functionality of the button 3888 of the remote 3860. In the illustrated embodiment, the first mode 3984a is a hold mode. In the hold mode, the aspiration valve 122 coupled to the aspiration conduit 121 is held in the open position so long as the button 3888 remains actuated (e.g., depressed) by the user to allow for continuous aspiration. In other embodiments, the hold mode can also open and/or close other valves to control fluid flow within the system 3400.

In the illustrated embodiment, the second mode 3984b is a continuous mode. In the continuous mode, the user can initiate aspiration of the aspiration catheter 102 by actuating the button 3888 one time to open the aspiration valve 122, and aspiration can continue indefinitely until the user stops the aspiration by actuating the button 3888 a second time. In some embodiments, such continuous aspiration can be automatically stopped if fluid in the chamber 3411 of the collection chamber 3410 fills to a predetermined level detected by the fluid level detector 3770 (FIGS. 37A and 37B).

In the illustrated embodiment, the third mode 3984c is a whoosh mode (e.g., a pre-charged mode, a built-up vacuum mode, a stored vacuum mode, etc.). In the whoosh mode, the user can aspirate a set volume of aspirated material in a single cycle. For example, pressing the button 3888 once can cause the system 3400 to generate a predetermined level of vacuum (e.g., 60 cc) within the chamber 3411 while the aspiration valve 122 is closed, and then open the aspiration valve 122 to apply the vacuum pressure built up in the chamber 3411 to the aspiration catheter 102. In other embodiments, the whoosh mode can correspond to different volumes of material, such as 20 cc, 40 cc, 80 cc, 100 cc, 150 cc, 200 cc, 300 cc, or greater, or any value therebetween.

In the illustrated embodiment, the mode selector 3681 also includes a button 3987. The button 3987 can be actuated to change the speed of aspiration. For example, when the button 3987 is not actuated (e.g., not depressed), the system 3400 can operate in a fast mode, and when the button 3987 is actuated (e.g., depressed), the system 3400 can operate in a slow mode (or vice versa). In some embodiments, the speed of aspiration in the fast mode is twice as fast as the speed of aspiration in the slow mode. In other embodiments, the ratio of speed of aspiration in the fast mode relative to the speed of aspiration in the slow mode can be 1.1:1, 1.5:1, 2.5:1, 3:1, 4:1, or any ratio therebetween. The speed of aspiration can be controlled in a variety of ways, including, for example, decreasing a diameter of the aspiration conduit 121 or the pressure conduit 3423 (FIG. 34) via a valve (not shown), changing the amount of pressure generated by the pump assembly 3440, opening a vent or valve (e.g., a three-way stopcock fluidly coupled to the aspiration conduit 121; not shown), and/or the like. In some embodiments, the system 3400 operates to open the aspiration valve 122 in the slow mode, effectively providing a smaller diameter choke point along the flow path from the aspiration catheter 102 to the chamber 3411 that acts to slow the application of vacuum pressure.

FIGS. 40A and 40B are top views of the mode selector 3681 of the clot treatment system 3400 of FIG. 34 in accordance with additional embodiments of the present technology. Referring to FIGS. 40A and 40B together, the second mode 3984b is selected, as indicated by the selection indicator 3986. More specifically, the selection indicator 3986 is aligned with the selected mode 3984. Referring to FIG. 40A, the button 3987 is not actuated (e.g., not depressed), and the system 3400 is in fast mode. The fast mode is indicated by the dark color (e.g., blue) of the recessed light 3983 and the selection indicator 3986. Referring to FIG. 40B, the button 3987 is actuated (e.g., depressed), and the system 3400 is in slow mode. The slow mode is indicated by the light color (e.g., green) of the recessed light 3983 and the selection indicator 3986. In other embodiments, the colors, intensity, pattern, etc. of the recessed light 3983 and/or the selection indicator 3986 can vary for each mode (e.g., the first mode 3984a, fast mode). For example, the recessed light 3983 and/or the selection indicator 3986 can be brighter in the fast mode than in the slow mode. In further embodiments, other components can be illuminated (e.g., the button 3987) and/or some of the components can be a constant color (e.g., the selection indicator 3986).

Referring to FIGS. 40A and 40B together, the modes 3984 are also illuminated based on whether the system 3400 is in fast mode or slow mode. Referring to FIG. 40A, the second mode 3984b is illuminated to indicate that the system 3400 is in fast mode. Referring to FIG. 40B, the second mode 3984b is not illuminated to indicate that the system 3400 is in slow mode. In other embodiments, the modes 3984 can be a constant color or brightness.

FIGS. 41A-41F are simplified side views of the portion of the clot treatment system 3400 of FIG. 34 during different aspiration and reinfusion states in accordance with additional embodiments of the present technology. Referring to FIGS. 41A-41F together, the collection chamber 3410 and the remote 3860 are shown together illuminated in various ways to indicate various system 3400 statuses. More specifically, the LED strip 3492 of the collection chamber 3410, the button 3688 of the electronics housing 3680, the LED strip 3862 of the remote 3860, and/or the button 3888 of the remote 3860 can be illuminated in different modes to indicate the different statuses of the system 3400. In other embodiments, other components of the system 3400 (e.g., the buttons 388, 488, 3987) can be illuminated to indicate the status of the system 3400. In further embodiments, different colors, intensities, patterns, and/or the like can be used to illuminate the system 3400 than the colors, intensities, and/or patterns described in detail below with reference to FIGS. 41A-41F. In other embodiments, different combinations of colors, intensities, patterns, illuminated components, and/or the like can be used to indicate other system 3400 statuses.

Referring to FIG. 41A, the LED strip 3492 of the collection chamber 3410 and the LED strip 3862 of the remote 3860 are illuminated in a swirling pattern (e.g., pulsing, selective illumination of individual LEDs in a circular pattern) of a first color (e.g., green). In some embodiments, this configuration can indicate that the pump assembly 3440 (FIG. 34) is charging a vacuum for aspiration of the aspiration catheter 102 (FIG. 1) in the chamber 3411. More specifically, the button 3888 is not illuminated to indicate to the user that actuating the button 3888 will not result in aspiration because the vacuum has not been fully charged. Further, the button 3688 of the electronics housing 3680, which can be configured to cause reinfusion when actuated, is not illuminated to indicate to the user that the system 3400 is not primed for reinfusion.

Referring to FIG. 41B, the LED strip 3492 of the collection chamber 3410, the LED strip 3862 of the remote 3860, and the button 3888 of the remote 3860 are constantly illuminated in a solid color (e.g., consistent brightness with no pulsing, swirling, or other visual effect), such as the same first color. In some embodiments, this configuration can indicate to the user that the vacuum is fully charged and that the system 3400 is ready to aspirate the aspiration catheter 102. More specifically, the button 3888 is illuminated to indicate to the user that actuating the button 3888 will result in aspiration because the vacuum is fully charged. Further, the button 3688 of the electronics housing 3680, which can be configured to cause reinfusion when actuated, is not illuminated to indicate to the user that the system 3400 is not primed for reinfusion. Referring to FIGS. 41A and 41B together, for example, the system 3400 can first display the illumination of FIG. 41A while the vacuum charges, and swap to the illumination of FIG. 41B when the vacuum is fully charged.

Referring to FIGS. 41C, the LED strip 3492 of the collection chamber 3410, the button 3688 of the electronics housing 3680, and the LED strip 3862 of the remote 3860 are illuminated in a blinking (e.g., pulsing) second color (e.g., blue). More specifically, the button 3888 of the remote 3860 is not illuminated to indicate to the user that aspiration is not currently possible. In some embodiments, this configuration can indicate to the user that the chamber 3411 of the collection chamber 3410 is full of aspirated material, clot material, filtered material, etc. For example, if the user attempted to actuate the button 3888 to aspirate the aspiration catheter 102, the system 3400 will not aspirate because the chamber 3411 is full, and the remote 3860 will vibrate to alert the user that aspiration is not currently possible. Further, in this state, the button 3688, which can be configured to cause reinfusion when actuated, is also blinking in the same second color to indicate to the user that the button 3688 should be actuated to cause reinfusion and reduce the fluid level in the chamber 3411.

Referring to FIGS. 41D, the LED strip 3492 of the collection chamber 3410, the button 3688 of the electronics housing 3680, and the LED strip 3862 of the remote 3860 are illuminated in a solid color (e.g., consistent brightness with no pulsing, swirling, or other visual effect), such as the same second color. More specifically, the button 3888 of the remote 3860 is not illuminated to again indicate to the user that aspiration is not currently possible. In some embodiments, this configuration can indicate to the user that reinfusion is occurring. In other embodiments, the reinfusion can include flushing the chamber 3411 with saline, as described in greater detail above with reference to FIGS. 33 and 36. For example, referring to FIGS. 41C and 41D together, the system 3400 can first display the illumination of FIG. 41C to prompt the user to initiate reinfusion and, once reinfusion is initiated by the user, swap to the illumination of FIG. 41D while reinfusion and/or flushing occurs.

Referring to FIG. 41E, the LED strip 3492 of the collection chamber 3410 and the LED strip 3862 of the remote 3860 are illuminated in a solid color (e.g., consistent brightness with no pulsing, swirling, or other visual effect), such as a different third color (e.g., yellow). More specifically, the button 3888 of the remote 3860 and the button 3688 of the electronics housing 3680 are not illuminated to indicate that both aspiration and reinfusion are not currently possible. In some embodiments, the yellow color can indicate to the user that there is an obstruction at an end of the aspiration catheter 102 (e.g., a clot “lollipop”) and/or in the aspiration conduit 121 that is preventing aspiration from occurring. In further embodiments, the presence of the obstruction can be determined by the fluid level detector 3770 (FIGS. 37A and 37B). More specifically, if the level of fluid within the chamber 3411 does not change after an aspiration cycle, it can be determined that an obstruction is present in the aspiration catheter 102 and/or the aspiration conduit 121 and preventing fluid from entering the chamber 3411.

Referring to FIG. 41F, the LED strip 3492 of the collection chamber 3410 and the LED strip 3862 of the remote 3860 are illuminated in a solid color (e.g., consistent brightness with no pulsing, swirling, or other visual effect), such as a different fourth color (e.g., red). More specifically, the button 3888 of the remote 3860 and the button 3688 of the electronics housing 3680 are not illuminated to indicate that both aspiration and reinfusion are not currently possible. In some embodiments, this state can indicate to the user that an air bubble has been detected in the reinfusion conduit 125 (e.g., via the bubble detector 164 of FIG. 36) and that it must be removed before operations can proceed.

The above illustrations in FIGS. 41A-41F are merely illustrative of different ways to convey status information to the user. In other embodiments, different colors, light patterns, etc., can be used to indicate the various states, more or fewer lights can be used, and/or different user-indications can supplement or replace the illustrated lights (e.g., tactile feedback, audio feedback, etc.).

### V. SELECTED EXAMPLES

The following examples are illustrative of several embodiments of the present technology:

1. A system for treating clot material in a vasculature of a patient, comprising:
an aspiration catheter defining an aspiration lumen and having a distal end portion, wherein the aspiration catheter is configured to be positioned within the vasculature of the patient such that the distal end portion is positioned proximate to the clot material;
a reinfusion catheter defining a reinfusion lumen, wherein the reinfusion catheter is configured to be positioned within the vasculature of the patient;
a collection chamber defining a chamber;
a filter positioned within the chamber;
a pump assembly including a pump having an inlet and an outlet, wherein the pump is configured to draw air through the inlet and drive the air out of the outlet;
a vacuum valve between the pump assembly and the collection chamber, wherein the vacuum valve is movable between an open position in which the inlet of the pump is fluidly connected to the chamber such that the pump generates negative pressure within the chamber and a closed position in which the inlet of the pump is fluidly disconnected from the chamber;
a positive pressure valve between the pump assembly and the collection chamber, wherein the positive pressure valve is movable between an open position in which the outlet of the pump is fluidly connected to the chamber such that the pump generates positive pressure within the chamber and a closed position in which the outlet of the pump is fluidly disconnected from the chamber;
an aspiration valve between the aspiration catheter and the collection chamber, wherein the aspiration valve is movable between an open position in which the aspiration lumen is fluidly connected to the chamber and a closed position in which the aspiration lumen is fluidly disconnected from the chamber; and
a reinfusion valve between the reinfusion catheter and the collection chamber, wherein the reinfusion valve is movable between an open position in which the reinfusion lumen is fluidly connected to the chamber and a closed position in which the reinfusion lumen is fluidly disconnected from the chamber.

2. The system of example 1, further comprising a control system communicatively coupled to the vacuum valve, the positive pressure valve, the aspiration valve, and the reinfusion valve, wherein the control system is configured to independently control each of the vacuum valve, the positive pressure valve, the aspiration valve, and the reinfusion valve to move between the open and closed positions.

3. The system of example 2 wherein the control system is configured to control each of the vacuum valve, the positive pressure valve, the aspiration valve, and the reinfusion valve in a sequence comprising:
closing each of the vacuum valve, the positive pressure valve, the aspiration valve, and the reinfusion valve;
opening the vacuum valve such that the pump generates the negative pressure within the chamber;
closing the vacuum valve;
opening the aspiration valve to apply at least a portion of the negative pressure to the aspiration lumen of the aspiration catheter to aspirate at least a portion of the clot material and blood through the aspiration lumen;
closing the aspiration valve; and
opening the positive pressure valve and the reinfusion valve such that the pump generates the positive pressure within the chamber to drive at least a portion of the blood from the chamber through the filter and into and at least partially through the reinfusion lumen for reinfusion into the vasculature of the patient, wherein the filter is configured to inhibit the portion of the clot material from passing through the filter to the reinfusion lumen.

4. The system of any one of examples 1-3, further comprising a fluid inlet valve, wherein the fluid inlet valve is movable between an open position in which the inlet of the pump is fluidly connected to atmosphere and a closed position in which the inlet of the pump is fluidly disconnected from the atmosphere.

5. The system of any one of examples 1-4, further comprising a fluid outlet valve, wherein the fluid outlet valve is movable between an open position in which the outlet of the pump is fluidly connected to atmosphere and a closed position in which the outlet of the pump is fluidly disconnected from the atmosphere.

6. The system of example 5 wherein the fluid outlet valve is configured to passively close when the positive pressure valve is in the open position.

7. The system of any one of examples 1-6 wherein the aspiration valve comprises an automated large stopcock valve.

8. The system of any one of examples 1-7 wherein:
the filter comprises a coarse filter and a fine filter, and
the coarse filter is positioned at a top of the chamber and the fine filter is positioned at a bottom of the chamber such that the aspiration catheter is fluidly connected to the chamber at a position upstream of the coarse filter and the reinfusion catheter is fluidly connected to the chamber at a position downstream the fine filter,
wherein, when the positive pressure valve is in the open position, the outlet of the pump is fluidly connected to the chamber upstream of the fine filter and downstream of the aspiration catheter, and
wherein, when the vacuum valve is in the open position, the inlet of the pump is fluidly connected to the chamber upstream the fine filter and downstream of the aspiration catheter.

9. The system of example 8 wherein, when the positive pressure valve is in the open position, the outlet of the pump is fluidly connected downstream of the coarse filter.

10. The system of example 8 wherein, when the vacuum valve is in the open position, the inlet of the pump is fluidly connected upstream of the coarse filter.

11. The system of example 10 wherein, when the positive pressure valve is in the open position, the outlet of the pump is fluidly connected upstream of the coarse filter.

12. The system of example 11, further comprising a one-way valve positioned between the coarse filter and the fine filter such that an upper chamber exists above the coarse filter and a lower chamber exists below the one-way valve.

13. The system of example 12, further comprising a vent positioned in fluid connection with the lower chamber.

14. The system of example 13 wherein the vent is connected to atmosphere or ambient pressure.

15. A system for aspirating and filtering clot material, comprising:
a housing;
a collection component spanning across the housing and dividing the housing into a first chamber and a second chamber, wherein the collection component includes a one-way valve positioned to (a) permit fluid flow from the first chamber to the second chamber and (b) inhibit fluid flow from the second chamber to the first chamber;
a positive pressure port extending through the housing and configured to fluidly couple the first chamber to a source of positive pressure;
a negative pressure port extending through the housing and configured to fluidly couple the first chamber to a source of negative pressure;
an aspiration port extending through the housing and configured to fluidly couple the first chamber to an aspiration catheter intravascularly positioned within a patient proximate to the clot material;
a reinfusion port extending through the housing and configured to fluidly couple the second chamber to a reinfusion source;
a first filter between the aspiration port and the one-way valve, wherein the first filter has a first porosity; and
a second filter between the one-way valve and the reinfusion port, wherein the second filter has a second porosity less than the first porosity.

16. The system of example 15, further comprising a vent port extending through the housing and configured to fluidly couple the second chamber to atmosphere and/or ambient pressure.

17. The system of example 15 or example 16 wherein the one-way valve is an umbrella valve.

18. The system of example 15 or example 16 wherein the one-way valve is a duckbill valve.

19. The system of example 15 or example 16 wherein the one-way valve is a cross-slit valve.

20. The system of example 15 or example 16 wherein the one-way valve is a ball valve.

21. The system of example 15 or example 16 wherein the one-way valve is a dome valve.

22. The system of any one of examples 15-21 wherein the first chamber is positioned above the second chamber.

23. The system of any one of examples 15-21 wherein the first chamber is positioned side-by-side with the second chamber.

24. The system of any one of examples 15-23 wherein the positive pressure port and the negative pressure port comprise a same port extending through the housing.

25 The system of any one of examples 15-24 wherein the reinfusion source is a reinfusion catheter intravascularly positioned within the patient.

26. The system of any one of examples 15-25 wherein the reinfusion source is a syringe.

27. The system of any one of examples 15-26 wherein:
the source of negative pressure is configured to generate negative pressure in the first chamber via the aspiration port; and
the one-way valve is configured to inhibit the negative pressure generated by the aspiration source from being applied to the second chamber.

28. The system of any one of examples 15-27 wherein:
the source of negative pressure is configured to generate negative pressure in the first chamber via the aspiration port to aspirate blood and at least a portion of the clot material into the first chamber, wherein the first filter is configured to filter larger portions of the clot material from the blood and smaller portions of the clot material;
the one-way valve is configured to inhibit the negative pressure generated by the aspiration source from being applied to the second chamber; and
the source of positive pressure is configured to generate positive pressure in the first chamber via the positive pressure port to drive the blood and the smaller portions of the clot material through the one-way valve from the first chamber to the second chamber.

29 The system of example 28 wherein the source of positive pressure is further configured to generate the positive pressure in the second chamber via the positive pressure port and the one-way valve to drive the blood through the second filter to the reinfusion source, and wherein the second filter is configured to filter the smaller portions of the clot material from the blood.

30. The system of any one of examples 15-29 wherein the source of negative pressure and the source of positive pressure comprise a same pump.

31. The system of any one of examples 15-29 wherein the source of negative pressure and the source of positive pressure comprise different pumps.

### VI. CONCLUSION

All numeric values are herein assumed to be modified by the term about whether or not explicitly indicated. The term about, in the context of numeric values, generally refers to a range of numbers that one of skill in the art would consider equivalent to the recited value (e.g., having the same function and/or result). For example, the term about can refer to the stated value plus or minus ten percent. For example, the use of the term about 100 can refer to a range of from 90 to 110, inclusive. In instances in which the context requires otherwise and/or relative terminology is used in reference to something that does not include, or is not related to, a numerical value, the terms are given their ordinary meaning to one skilled in the art.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A system for treating clot material in a vasculature of a patient, comprising:
an aspiration catheter defining an aspiration lumen and having a distal end portion, wherein the aspiration catheter is configured to be positioned within the vasculature of the patient such that the distal end portion is positioned proximate to the clot material;
a reinfusion catheter defining a reinfusion lumen, wherein the reinfusion catheter is configured to be positioned within the vasculature of the patient;
a collection chamber defining a chamber;
a filter positioned within the chamber;
a pump assembly including a pump having an inlet and an outlet, wherein the pump is configured to draw air through the inlet and drive the air out of the outlet;
a vacuum valve between the pump assembly and the collection chamber, wherein the vacuum valve is movable between an open position in which the inlet of the pump is fluidly connected to the chamber such that the pump generates negative pressure within the chamber and a closed position in which the inlet of the pump is fluidly disconnected from the chamber;
a positive pressure valve between the pump assembly and the collection chamber, wherein the positive pressure valve is movable between an open position in which the outlet of the pump is fluidly connected to the chamber such that the pump generates positive pressure within the chamber and a closed position in which the outlet of the pump is fluidly disconnected from the chamber;
an aspiration valve between the aspiration catheter and the collection chamber, wherein the aspiration valve is movable between an open position in which the aspiration lumen is fluidly connected to the chamber and a closed position in which the aspiration lumen is fluidly disconnected from the chamber; and
a reinfusion valve between the reinfusion catheter and the collection chamber, wherein the reinfusion valve is movable between an open position in which the reinfusion lumen is fluidly connected to the chamber and a closed position in which the reinfusion lumen is fluidly disconnected from the chamber.

2. The system of claim 1, further comprising a control system communicatively coupled to the vacuum valve, the positive pressure valve, the aspiration valve, and the reinfusion valve, wherein the control system is configured to independently control each of the vacuum valve, the positive pressure valve, the aspiration valve, and the reinfusion valve to move between the open and closed positions.

3. The system of claim 2 wherein the control system is configured to control each of the vacuum valve, the positive pressure valve, the aspiration valve, and the reinfusion valve in a sequence comprising:
closing each of the vacuum valve, the positive pressure valve, the aspiration valve, and the reinfusion valve;
opening the vacuum valve such that the pump generates the negative pressure within the chamber;
closing the vacuum valve;
opening the aspiration valve to apply at least a portion of the negative pressure to the aspiration lumen of the aspiration catheter to aspirate at least a portion of the clot material and blood through the aspiration lumen;
closing the aspiration valve; and
opening the positive pressure valve and the reinfusion valve such that the pump generates the positive pressure within the chamber to drive at least a portion of the blood from the chamber through the filter and into and at least partially through the reinfusion lumen for reinfusion into the vasculature of the patient, wherein the filter is configured to inhibit the portion of the clot material from passing through the filter to the reinfusion lumen.

4. The system of claim 1, further comprising a fluid inlet valve, wherein the fluid inlet valve is movable between an open position in which the inlet of the pump is fluidly connected to atmosphere and a closed position in which the inlet of the pump is fluidly disconnected from the atmosphere.

5. The system of claim 1, further comprising a fluid outlet valve, wherein the fluid outlet valve is movable between an open position in which the outlet of the pump is fluidly connected to atmosphere and a closed position in which the outlet of the pump is fluidly disconnected from the atmosphere.

6. The system of claim 5 wherein the fluid outlet valve is configured to passively close when the positive pressure valve is in the open position.

7. The system of claim 1 wherein the aspiration valve comprises an automated large stopcock valve.

8. The system of claim 1 wherein:
the filter comprises a coarse filter and a fine filter, and
the coarse filter is positioned at a top of the chamber and the fine filter is positioned at a bottom of the chamber such that the aspiration catheter is fluidly connected to the chamber at a position upstream of the coarse filter and the reinfusion catheter is fluidly connected to the chamber at a position downstream the fine filter,
wherein, when the positive pressure valve is in the open position, the outlet of the pump is fluidly connected to the chamber upstream of the fine filter and downstream of the aspiration catheter, and
wherein, when the vacuum valve is in the open position, the inlet of the pump is fluidly connected to the chamber upstream the fine filter and downstream of the aspiration catheter.

9. The system of claim 8 wherein, when the positive pressure valve is in the open position, the outlet of the pump is fluidly connected downstream of the coarse filter.

10. The system of claim 8 wherein, when the vacuum valve is in the open position, the inlet of the pump is fluidly connected upstream of the coarse filter.

11. The system of claim 10 wherein, when the positive pressure valve is in the open position, the outlet of the pump is fluidly connected upstream of the coarse filter.

12. The system of claim 11, further comprising a one-way valve positioned between the coarse filter and the fine filter such that an upper chamber exists above the coarse filter and a lower chamber exists below the one-way valve.

13. The system of claim 12, further comprising a vent positioned in fluid connection with the lower chamber.

14. The system of claim 13 wherein the vent is connected to atmosphere or ambient pressure.

15. A system for aspirating and filtering clot material, comprising:
a housing;
a collection component spanning across the housing and dividing the housing into a first chamber and a second chamber, wherein the collection component includes a one-way valve positioned to (a) permit fluid flow from the first chamber to the second chamber and (b) inhibit fluid flow from the second chamber to the first chamber;
a positive pressure port extending through the housing and configured to fluidly couple the first chamber to a source of positive pressure;
a negative pressure port extending through the housing and configured to fluidly couple the first chamber to a source of negative pressure;
an aspiration port extending through the housing and configured to fluidly couple the first chamber to an aspiration catheter intravascularly positioned within a patient proximate to the clot material;
a reinfusion port extending through the housing and configured to fluidly couple the second chamber to a reinfusion source;
a first filter between the aspiration port and the one-way valve, wherein the first filter has a first porosity; and
a second filter between the one-way valve and the reinfusion port, wherein the second filter has a second porosity less than the first porosity.

16. The system of claim 15, further comprising a vent port extending through the housing and configured to fluidly couple the second chamber to atmosphere and/or ambient pressure.

17. The system of claim 15 wherein the first chamber is positioned above the second chamber.

18. The system of claim 15 wherein the first chamber is positioned side-by-side with the second chamber.

19. The system of claim 15 wherein the positive pressure port and the negative pressure port comprise a same port extending through the housing.

20. The system of claim 15 wherein the reinfusion source is a reinfusion catheter intravascularly positioned within the patient.

21. The system of claim 15 wherein the reinfusion source is a syringe.

22. The system of claim 15 wherein:
the source of negative pressure is configured to generate negative pressure in the first chamber via the aspiration port; and
the one-way valve is configured to inhibit the negative pressure generated by the aspiration source from being applied to the second chamber.

23. The system of claim 15 wherein:
the source of negative pressure is configured to generate negative pressure in the first chamber via the aspiration port to aspirate blood and at least a portion of the clot material into the first chamber, wherein the first filter is configured to filter larger portions of the clot material from the blood and smaller portions of the clot material;
the one-way valve is configured to inhibit the negative pressure generated by the aspiration source from being applied to the second chamber; and
the source of positive pressure is configured to generate positive pressure in the first chamber via the positive pressure port to drive the blood and the smaller portions of the clot material through the one-way valve from the first chamber to the second chamber.

24. The system of claim 23 wherein the source of positive pressure is further configured to generate the positive pressure in the second chamber via the positive pressure port and the one-way valve to drive the blood through the second filter to the reinfusion source, and wherein the second filter is configured to filter the smaller portions of the clot material from the blood.

25. The system of claim 15 wherein the source of negative pressure and the source of positive pressure comprise a same pump.

26. The system of claim 15 wherein the source of negative pressure and the source of positive pressure comprise different pumps.
